(19) United States
(12) Patent Application Publication
(10) Pub. No.: US 2024/0139301 A1
(43) Pub. Date: May 2, 2024

(54) ACTIVE CANCER IMMUNOTHERAPY BY IMMUNE MODULATION VIA GLOBO SERIES ANTIGENS

(71) Applicant: OBI Pharma, Inc., Taipei City (TW)

(72) Inventors: Ming-Tain LAI, Taipei City (TW); Cheng-Der Tony YU, Taipei City (TW); I-Ju CHEN, Taipei City (TW); Wei-Han LEE, Taipei City (TW); Chueh-Hao YANG, Taipei City (TW); Chun-Yen TSAO, Taipei City (TW); Chang-Lin HSIEH, Taipei City (TW); Chien-Chih OU, Taipei City (TW); Chen-En TSAI, Taipei City (TW)

(21) Appl. No.: 18/253,576

(22) PCT Filed: Nov. 19, 2021
(86) PCT No.: PCT/US21/72513

§ 371 Date: May 18, 2023

Related U.S. Application Data
(60) Provisional application No. 63/116,015, filed on Nov. 19, 2020.

(51) Int. Cl. A61K 39/00; A61K 45/06; A61K 47/64; A61P 35/00

(52) U.S. Cl. CPC A61K 39/001173; A61K 45/06; A61K 47/646; A61P 35/00; A61K 2039/545; A61K 2039/55544; A61K 2039/86

(57) ABSTRACT

The disclosure provides a method of active immunotherapy for a cancer patient, comprising administering vaccines against Globo series antigens (i.e., Globo H, SSEA-3 and SSEA-4). Specifically, the method comprises administering Globo H-CRM197 (OBI-833/821) in patients with cancer. The disclosure also provides a method of selecting a cancer patient who is suitable as treatment candidate for immunotherapy. Exemplary immune response can be characterized by reduction of the severity of disease, including but not limited to, prevention of disease, delay in onset of disease, decreased severity of symptoms, decreased morbidity and delayed mortality.

### PRIORITY DATA

This application is a 371 National Phase of International Patent Application No.: PCT/US21/72513, filed on Nov. 19, 2021, which claims the benefit and priority to U.S. Provisional Application No. 63/116,015, filed Nov. 19, 2020, the contents of which are incorporated by reference herewith in their entirety.

### FIELD OF THE INVENTION

This invention relates to methods for immunotherapy of a cancer patient comprising administering to the patient immunotherapeutic agents against Globo series antigens (Globo H, SSEA-4 and SSEA-3).

### BACKGROUND OF THE INVENTION

Cancer Immunotherapy

Recent advances in the aspects of cellular immunology and tumor-host immune interactions, have led to the development of effective immune-based therapies capable of reducing tumor size in patients with metastatic cancer. Currently, there are three main approaches to cancer immunotherapy, a non-specific stimulation of immune reactions by stimulating effector cells and/or inhibiting regulatory cells, an active immunization to enhance specific anti-tumor reactions, known as cancer vaccines, and a passive transfer of anti-tumor antibodies or activated immune cells with antitumor activity, also known as adoptive immunotherapy (DeVita et al. 2008).

Cancer immunotherapy is based on the theory that tumor specific antigens can be recognized when processed by, and presented to, a properly trained immune system. The malignant cells are commonly characterized by the appearance of large and unusual protein and carbohydrate motifs on their cell surfaces which distinguish them from their normal cell counterparts (Rabinovich et al. 1994). The identification of tumor-associated antigens recognized by cellular or humoral effectors of the immune system has opened new perspectives for cancer therapy. In the past two decades several monoclonal antibodies have provided sufficient efficacy and safety data to gain regulatory approval for passive immunotherapy of cancer. These include edrecolomab (Mab 17-1A), rituximab (anti-CD20), trastuzumab (Herceptin), gemtuzamab zogamicin (Mylotarg), and alemtuzumab (CAMPATH-1, anti-CD52).

Recently, ch14.18, a chimeric monoclonal antibody against GD2, has been shown to improve survival of high risk neuroblastoma patients in a Phase III trial, making this the first effective anti-cancer antibody targeting carbohydrate antigens (Yu et al. 2010). In addition to passive immunotherapy with these monoclonal antibodies, active immunotherapy of human cancer is a rapidly growing experimental area targeting peptide as well as carbohydrate tumor-associated antigens. The biotechnology and pharmaceutical industries have only recently successfully developed cancer vaccines. In 2006, Gardasil was approved in the U.S, and it is the first cancer preventive vaccine against the human papillomavirus that causes cervical cancer and genital warts. In July 2007, Northwest Biotherapeutics received approval of its personalized therapeutic vaccine for brain cancer (DCVax®-Brain) in Switzerland. In April 2009, the Russian Ministry of Public Health issued a registration certificate for the use of Oncophage® (vitespen) in the treatment of kidney cancer patients at intermediate risk for disease recurrence (http://www.antigenics.com/news/2008/0408.phtml). These successful cases are encouraging in reaffirming the development of Globo H as an immunotherapeutic target for solid tumors such as breast, gastric, lung, colorectal, ovarian, pancreatic and possibly other cancer types.

In adjuvant immunization trials, the primary target is the “minimal residual disease” which consists of a small number of tumor cells or early micrometastases that may persist for long periods after apparent resection of all residual tumors (Zhang et al. 1996; Zhang et al. 1997a; Zhang et al. 1997b; Zhang et al. 1998). Active immunotherapy with therapeutic vaccines is a good strategy to target minimal residual disease and prevent relapse by inducing antibodies of sufficient titer against tumor antigens to eliminate residual tumor cells from the peritoneal cavity, blood and lymphatic systems, and to inhibit micrometastases. However, the immune response to cancer antigens is suppressed in cancer patients (Sotomayor et al. 1996; Pawelec et al. 1997; Khong and Restifo 2002). In the past few years, methods have been developed to synthesize antigens that mimic natural cancer antigens (Musselli et al. 2001) which are conjugated to a potent immunogen as a carrier and co-administered with an immuno-stimulatory molecule as an adjuvant that can reliably provoke an immune response.

Many of the more tumor-restricted monoclonal antibodies derived by immunization of mice with human tumor cells have been directed against carbohydrate antigens expressed at the cell surface (Menard et al. 1983; Zhang et al. 1997a). Cell surface carbohydrates are characteristic of different stages of normal development and differentiation; distinct carbohydrates are expressed in tissue- and cell-specific manners during those processes. Several carbohydrate antigens (Livingston 1995a; Livingston et al. 1997; Livingston and Ragupathi 1997) have proven to be promising targets for immunotherapy. Immunization against carbohydrate antigens conjugated to immuno-stimulatory molecules results in humoral antibody response, primarily an IgM antibody response. These antibodies are known to induce complement dependent cytotoxicity (CDC), inflammation, and phagocytosis of tumor cells by the reticulo-endothelial system (opsonization).

In addition to CDC, IgG antibodies of subclasses IgG1 and IgG3 in humans can also induce antibody dependent cell mediated cytotoxicity (ADCC). They are ideally suited for eradication of residual tumor cells and systemic or intraperitoneal micrometastases which have been well-documented in a variety of mouse experiments. Carbohydrate antigens have been chosen as targets for active immunotherapy include Globo H (Ragupathi et al. 1997; Slovin et al. 1999; Allen et al. 2001; Gilewski et al. 2001), GM2 (White et al. 1991; Livingston et al. 1994a; Livingston et al. 1994b; Livingston 1995b; Helling et al. 1995; Chapman et al. 2000a; Chapman et al. 2000b), GD2 (Livingston 1998), GD3 (Helling et al. 1994; McCaffery et al. 1996; Ragupathi et al. 2000), sTn (MacLean et al. 1993; Longenecker et al. 1993; Longenecker et al. 1994; Sandmaier et al. 1999) and Tn (Allen et al. 2001).

Sialyl-Tn (sTn) is a disaccharide tumor associated antigen expressed on the MUC1 mucin on a number of human cancer cells and is associated with more aggressive disease. Theratope® vaccine (Biomira, Inc., Edmonton, Alberta, Canada and Merck KGaA of Darmstadt, Germany) is a cancer vaccine which consists of a synthetic STn antigen conjugated to KLH (Keyhole limpet hemocyanin) combined with an immunostimulant (adjuvant) Detox™ derived from bacteria. A Phase III trial of Theratope® vaccine (sTn-KLH) vs KLH in 1,030 women with metastatic breast cancer showed that Theratope® vaccine was well tolerated with minimal toxicity. The most common adverse effects were induration and erythema at the site of injection. While the results of this trial did not meet the primary endpoints of time to disease progression and overall survival, a subsequent post-hoc analysis showed that women who received concurrent endocrine and Theratope® vaccine treatment had a significant overall survival benefit versus those who received endocrine therapy alone (Ibrahim et al. 2013).

Useful glycans of the invention include tumor-associated carbohydrate antigens (TACAs). The cell-surface glycosphingolipid (GSL) Globo H is a member of a family of antigenic carbohydrates that are highly expressed on a range of cancer cell lines. Other Globo H glycosphingolipid analogues useful in the present invention can be SSEA-3 (or Gb5), SSEA-4, Gb3 or Gb4. FIG. 1(A) indicated GSL roles in signaling, cellular cross-talk, and cell adhesion. Furthermore, FIG. 1(B) showed the synthesis pathway of Globo series antigens (Zhang et al. 2019).

Globo H Expression in Solid Tumors

Cancer cells contain unique tumor associated carbohydrate antigens (TACAs) that are not common to most of the host cell surfaces. The expression level of cell surface carbohydrate antigens is often significantly increased on carcinogenic transformation (Zhang et al. 1997a; Zhang et al. 1997b). Thus, TACAs offer the potential for a targeted immunotherapeutic approach to the treatment of certain forms of cancer. Among TACAs, great interest has been focused on the immunogenic potential of Globo H, the terminal hexasaccharide portion of the glycolipid. Globo H is highly expressed in epithelial cancers such as breast cancer, ovarian cancer, endometrial cancer, gastric cancer, colon cancer, pancreatic cancer, lung cancer, and prostate cancer (Zhang et al. 1997b); it is expressed on the cancer cell surface as a glycolipid and possibly as a glycoprotein (Miotti et al. 1989). FIG. 2 indicates Globo H plays an essential role on carcinogenic transformation, such as promoting immunosuppression, angiogenesis, or tumor survival signaling.

Globo H is also expressed at lower levels on some normal luminal surfaces (Zhang et al. 1998). However, the antigen is predominantly localized to the apical cells at secretory borders-sites that appear to be inaccessible to immune surveillance. In Phase I trials discussed below, Globo H antigen expressed at the secretory borders of normal epithelial tissues induced neither tolerance nor autoimmunity once antibodies were elicited, suggesting that the antigens are sequestered from the immune system.

Recent work has shown that Globo H and the Globo series Stage-Specific Embryonic Antigen 3 (SSEA-3) and Stage-Specific Embryonic Antigen 4 (SSEA-4) are expressed on epithelial cancer cells and corresponding cancerous stem cells (Chang et al. 2008b; Lou et al. 2014). SSEA-3 (Gb5) is a penta-saccharide precursor of Globo H, and is also known as stage-specific embryonic antigen SSEA-3. Until recently, SSEA-3 and SSEA-4 have been known as markers for human embryonic stem cells that can be observed only in stem cells during the embryonic development stage (Chang et al. 2008b; Lou et al. 2014). Finding of Globo H, SSEA-3 and SSEA-4 on cancer stem cells suggests that, in principle, the Globo H protein-conjugated immunotherapy not only targets the cancer cells, but also the cancer stem cells, for eradication. The same study also revealed Globo H expression in 25/41 breast cancer specimens (61.0%) and SSEA-3 expression in 31/40 (77.5%) various tumors (Chang et el. 2008a). FIG. 3 indicated Globo H is expressed on multiple epithelial cancer cell lines and the Globo H expression levels was from 25% to 100%.

It is noteworthy that similar to Globo H, SSEA-3 and SSEA-4 expression in normal tissues was predominantly at the secretory borders of epithelium (Chang et al. 2008b) where access to the immune system is restricted. Immunization of mice with Globo H-KLH and Globo H-CRM197 induced antibodies reactive with Globo H, SSEA-3, and SSEA-4, suggesting that a Globo H-based vaccine will target tumor cells expressing Globo H, SSEA-3, and SSEA-4 (Huang et al. 2013).

Recent data have shown that Globo H, SSEA-3 and SSEA-4 (so-called Globo Series TACAs) are highly expressed on human cancer specimens from various solid tumor types. These data show Globo H expression in 73/80 breast cancer specimens (91.3%), SSEA-3 expression in 79/79 (100%) tumors, and SSEA-4 expression in 80/80 (100%) (Dr. A. Yu, Taiwan unpublished data). This research also showed Globo H expression in 71/74 (95.9%), SSEA-3 expression in 76/79 (96.2%), and SSEA-4 expression in 72/81 (88.9%) gastric cancer specimens. In addition, the data also revealed high Globo series expression in lung cancer samples with Globo H expression in 61/61 (100%), SSEA-3 expression in 62/63 (98.4%), and SSEA-4 expression in 61/62 (98.4%) of the samples analyzed. (Data submitted for publication by Dr. A. Yu at Chang Gung Memorial Hospital, Taiwan)

Globo H expression was detected in 88/134 (65.7%), SSEA-3 expression in 38/134 (28.4%), and SSEA-4 expression in 96/134 (71.6%) of 134 human cancer cell lines from 14 tumor types, including Globo H expression in 14/23 (60.9%) breast cancer, 13/20 (65.0%) lung cancer, 6/6 (100%) gastric cancer, 6/7 (85.7%) colon cancer, 6/8 (75.0%) pancreatic cancer, and 2/2 (100%) esophageal cancer cell lines (Lou et al. 2014).

In this phase 1 clinical trial, the proposed plan is to enroll subjects with advanced/metastatic incurable gastric, breast, colorectal or lung cancer and subsequently evaluate the expression levels in the Globo series TACAs.

### SUMMARY OF THE INVENTION

Accordingly, the present disclosure is based on the discovery that Globo series antigens are aberrantly expressed in a broad spectrum of cancers, but not on normal cells. Cancers expressing Globo series antigens include, but are not limited to, sarcoma, skin cancer, leukemia, lymphoma, brain cancer, glioblastoma, lung cancer, breast cancer, oral cancer, head-and-neck cancer, nasopharyngeal cancer, esophageal cancer, stomach cancer, liver cancer, bile duct cancer, gallbladder cancer, bladder cancer, pancreatic cancer, intestinal cancer, colorectal cancer, kidney cancer, cervix cancer, endometrial cancer, ovarian cancer, testical cancer, buccal cancer, oropharyngeal cancer, laryngeal cancer and prostate cancer.

Aspects and embodiments of the present disclosure provide methods for treating a subject afflicted with cancer by immunotherapy comprising administering to the subject in need thereof a Globo series antigen-targeting immunogenic agent (e.g., OBI-833/OBI-821) useful for inducing/modulating an immune response (IgG and/or IgM), said method comprising improving the survival (including overall survival and/or progression free survival) by modulating Globo series antigen interaction, such that survival of the subject is improved. The therapeutic compositions are in part envisaged to act as cancer vaccines for boosting the body's natural ability to protect itself, through the immune system from dangers posed by damaged or abnormal cells such as cancer cells.

In one aspect, the disclosure provides a method for treating advanced/metastatic gastric, lung, colorectal, or breast cancer in a subject comprising administering to the subject in need thereof a therapeutically effective dose of Globo series antigen vaccine and/or cross-reacting with Globo series antigen vaccine.

In one embodiment, the disclosure provides a method wherein the Globo series antigen vaccine comprises Globo series antigens conjugated with a carrier protein.

In one embodiment, the disclosure provides a method wherein the Globo series antigen selected is Globo H.

In one embodiment, the disclosure provides a method wherein the carrier protein comprises DT-CRM 197 (diphtheria toxin cross-reacting material 197).

In one embodiment, the disclosure provides a method wherein the Globo series antigens vaccine is administered as a pharmaceutical composition.

In one embodiment, the disclosure provides a method wherein the pharmaceutical composition comprises OBI-833/OBI-821.

In one embodiment, the immunogenic agent can include OBI-833 and related variants.

In certain embodiments, the immune response can include: IgG (including subclasses IgG1, IgG2, IgG3, IgG4), IgM, CTLs (cytotoxic lymphocytes) directed to Globo H series antigens/tumors.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Schematic description of Globo series antigen. (A) A unique class of glycosphingolipids (GSLs) involved in tumor development and survival. (B) Globo series antigen. Stage-specific expression during early embryogenesis, markers for pluripotent embryonic SCs, and associated with EMT.

FIG. 2. Schematic illustration of how Globo H plays an essential role in cancer survival and progression.

FIG. 3. Schematic illustration of Globo H expression level in different cancers. (A) The Globo H expression ratio in fourteen cancer types. (B) The description of Globo H expression in lung cancer, esophageal cancer and triple-negative breast cancer (TNBC).

FIG. 4. Schematic illustration of OBI-833 (Globo H cancer vaccine).

FIG. 5. Schematic illustration of Globo H targeting with OBI-833

FIG. 6. Schematic illustration of OBI-833/OBI-821 study drug mixing instruction. (A) OBI-833 10 μg+OBI-821 100 μg; (B) OBI-833 30 μg+OBI-821 100 μg; (C) OBI-833 100 μg+OBI-821 100 μg.

FIG. 7. OBI-833 Phase 1 clinical trial design (OBI-833-001 study)

FIG. 8. Schematic illustration of OBI-833-001 CDC results. (A) Dose escalation phase. Cohort 1: OBI-833 (10 μg Globo H/100 μg OBI-821). Cohort 2: OBI-833 (30 μg Globo H/100 μg OBI-821). Cohort 3: OBI-833 (100 μg Globo H/100 μg OBI-821). (B) Cohort expansion phase (30 μg Globo H/100 μg OBI-821).

FIG. 9. Schematic illustration of OBI-833-001 ADCC results. (A) Dose escalation phase. Cohort 1: OBI-833 (10 μg Globo H/100 μg OBI-821). Cohort 2: OBI-833 (30 μg Globo H/100 μg OBI-821). Cohort 3: OBI-833 (100 μg Globo H/100 μg OBI-821). (B) Cohort expansion phase (30 μg Globo H/100 μg OBI-821).

FIG. 10. Immune response in OBI-833-001 dose escalation phase. (A) Anti-Globo H IgM response (B) Anti-Globo H IgG response. Cohort 1: OBI-833 (10 μg Globo H/100 μg OBI-821). Cohort 2: OBI-833 (30 μg Globo H/100 μg OBI-821). Cohort 3: OBI-833 (100 μg Globo H/100 μg OBI-821).

FIG. 11. Globo H expression in lung cancer (NSCLC) cohort patients. Th calculation of H-score is: (percentage cells with 1+intensity)×1+(percentage cells with 2+intensity)×2+(percentage cells with 3+intensity)×3. The range of H-score is from 0-300.

FIG. 12. Immune response in OBI-833-001 cohort expansion phase (30 μg Globo H/100 μg OBI-821). (A) Anti-Globo H IgM response (B) Anti-Globo H IgG response.

FIG. 13. Immune response in OBI-833-001 cohort expansion phase (30 μg Globo H/100 μg OBI-821). (A) Anti-SSEA3 IgM response (B) Anti-SSEA3 IgG response.

FIG. 14. Immune response in OBI-833-001 cohort expansion phase (30 μg Globo H/100 μg OBI-821). (A) Anti-SSEA4 IgM response (B) Anti-SSEA4 IgG response.

FIG. 15. Schematic illustration of Kaplan-Meier plot for the overall survival (OS) in OBI-833-001 dose escalation phase and cohort expansion phase.

FIG. 16. Schematic illustration of Kaplan-Meier plot for the progression-free survival (PFS) in OBI-833-001 dose escalation phase and cohort expansion phase.

FIG. 17. Schematic illustration of Swimmer Plot on EGFR TKls and OBI-833 treatment duration.

FIG. 18. Schematic illustration of tumor response time of NSCLC patients.

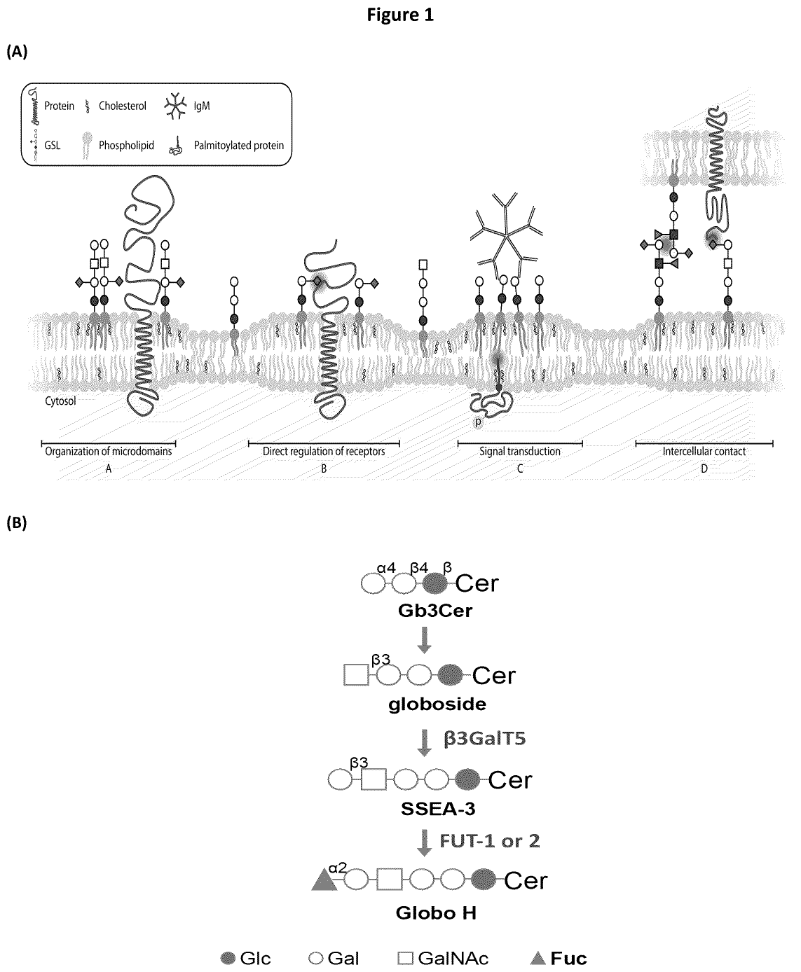

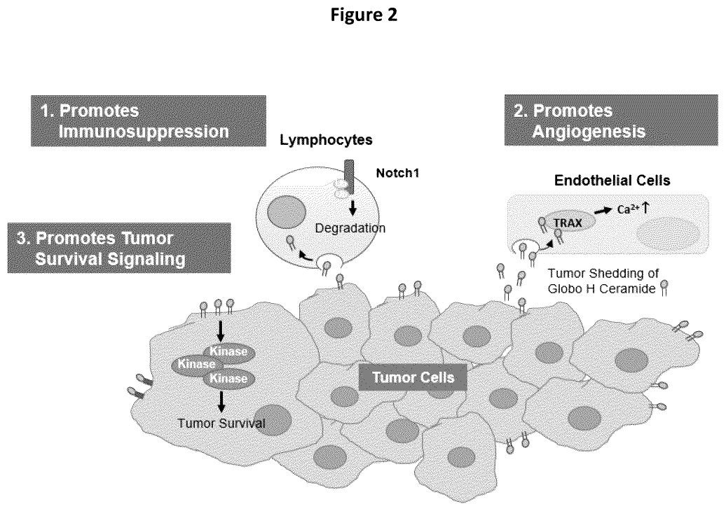

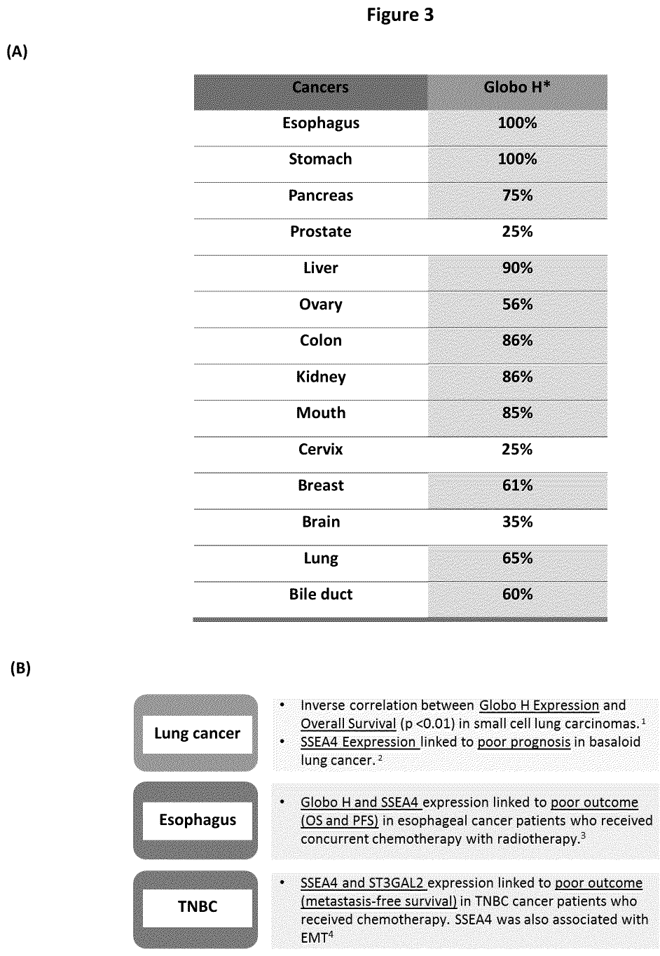

### DETAILED DESCRIPTION OF THE INVENTION

The present invention relates to methods for immunotherapy of a subject afflicted with diseases such as cancer or an infectious disease, which methods comprise administering to the subject a composition comprising a therapeutically effective amount of a compound or agent that potentiates an endogenous immune response, either stimulating the activation of the endogenous response or inhibiting the suppression of the endogenous response. More specifically, this disclosure provides methods for potentiating an endogenous immune response in a subject afflicted with cancer so as to thereby treat the patient, which method comprises administering to the subject a therapeutically effective amount of an immunogenic agent.

### List of Abbreviations and Definition of Terms

ADCC: antibody-dependent cell-mediated cytotoxicity
AE: adverse event
ALC: absolute lymphocyte count
ALK: Anaplastic Lymphoma Kinase
ALT (SGPT): serum glutamic pyruvic transaminase
ANCA: antineutrophil cytoplasmic antibody
AST (SGOT): serum glutamic oxaloacetic transaminase
BUN: blood urea nitrogen
CDC: complement dependent cytotoxicity
CA: cancer antigen
CEA: carcinoembryonic antigen
CNS: central nervous system
CR: complete response
CRF: case report form
CRM197: active and nontoxic form of diphtheria toxin (DT) called cross-reacting material 197
CT: computerized tomography
CTC: circulating tumor cell
CTCAE: common terminology criteria for adverse events
CYFRA 21-1: cytokeratin fragment 21-1
DLT: dose limiting toxicity
ECG: electrocardiogram
ECOG: Eastern Cooperative Oncology Group EF ejection fraction
EGFR: Epidermal Growth Factor Receptor
ESR: erythrocyte sedimentation rate
FACS: fluorescence-activated cell sorting
FDA: Food and Drug Administration
Gb5: Globo H precursor Gb5, also called stage specific embryonic antigen-3 (SSEA-3)
GCSF: granulocyte colony stimulating factor
GM-CSF: granulocyte macrophage colony stimulating factor HBV hepatitis B virus
HCC: hepatocellular carcinoma
HCV: hepatitis B virus
HIV: human immunodeficiency virus
ICF: informed consent form
ICH-GCP: International Conference of Harmonization—Good Clinical Practice IHC
Immunohistochemistry
IRB: Institutional Review Broad
KLH: keyhole limpet hemocyanin
LDH: lactate dehydrogenase
LVEF: left ventricular ejection fraction
mAbs: monoclonal antibodies
MRI: magnetic resonance imaging
MSKCC: Memorial-Sloan Kettering Cancer Center
MUGA: multiple gated acquisition scan.
NCI: National Cancer Institute
NK cell: natural killer cell
NKT cell: natural killer T cell
NOAEL: no observed adverse effect level
NSCLC: non-small cell lung cancer
OS: overall survival
PBS: phosphate buffer saline
PD: progressive disease
PD-1: programmed death-1
PD-L1: programmed death-ligand 1
PET: positron emission tomography
PR: partial response
RBC: red blood cell counts
RECIST: response evaluation criteria in solid tumors
RF: rheumatoid factor
SAEs: serious adverse event(s)
SC: subcutaneous
SD: stable disease
SOP: standard operating procedure
SSEA: stage specific embryonic antigen
SSEA-3: Globo H precursor Gb5, also called stage specific embryonic antigen-3
SSEA-4: sialyl Gb5, also called stage specific embryonic antigen-4
TKI: Tyrosine Kinase Inhibitor
ULN: upper limit of normal
WBC: white blood cell counts
WHO: World Health Organization

### Definitions

“Baseline values” means the baseline value for each parameter is defined as the last non-missing assessment prior to the first dose of study drug.

“End of study” means twelve weeks (Dose Escalation Phase) or 24 weeks (Cohort Expansion Phase) after the last subcutaneous dose of the investigational drug.

“Enrolled subjects” means all subjects who were found eligible to participate in the clinical study, signed an ICF, and could be screened for eligibility.

“Screened subjects” means all subjects who signed an ICF and underwent screening assessments to check whether or not they are eligible to participate in the clinical study.

“Investigational medicinal product (IMP)” means “A pharmaceutical form of an active substance or placebo being tested or used as a reference in a clinical trial, including products already with a marketing authorization but used or assembled (formulated or packaged) in a way different from the authorized form, or when used for an unauthorized indication, or when used to gain further information about the authorized form” [per EC Directive 2001/20/EC definition of IMP in Article 2 (d)].

“Administering” as used herein, means embodiments of an administration regimen that can include the following features: 1) Administer vaccine two or more times (e.g., 3, 4, 5, 6, 7, 8, 9, 10 or more times); 2) Each administration increases immune response (see above) [titer—IgG and/or IgM Ab amount, and/or increases affinity/avidity; induction of Abs to less immunogenic sites of Globo H portion of the Globo H antigen-conjugate (e.g., portions of Globo H antigen that may be less accessible in the conjugate)].

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing or decreasing inflammation and/or tissue/organ damage, decreasing the rate of disease progression, amelioration or palliation of the disease state, and remission or improved prognosis. In some embodiments, antibodies of the invention are used to delay development of a disease or disorder.

An “effective amount” refers to an amount effective, at dosages and for periods of time necessary, to achieve the desired therapeutic or prophylactic result.

A “therapeutically effective amount” of a substance/molecule of the invention may vary according to factors such as the disease state, age, sex, and weight of the individual, and the ability of the substance/molecule, to elicit a desired response in the individual. A therapeutically effective amount is also one in which any toxic or detrimental effects of the substance/molecule are outweighed by the therapeutically beneficial effects. A “prophylactically effective amount” refers to an amount effective at dosages and for periods of time necessary to achieve the desired prophylactic result. Typically, but not necessarily, since a prophylactic dose is used in subjects prior to or at an earlier stage of disease, the prophylactically effective amount would be less than the therapeutically effective amount.

“Adverse Event” (AE) Toxicity will be measured according to US NCI Common Toxicity Criteria, Version 4, developed by the Cancer Therapy Evaluation Program at the National Cancer Institute. The criteria for unacceptable toxicities should include any ≥Grade 4 toxicity, with the exception of local skin reactions, fever, chilling, sweats, urticaria, and/or pruritis since these are common side effects of antibody/adjuvant administration, are reversible, and controlled by supportive management. Theoretically, immune complex disease as manifested by skin, joint, renal, or other manifestations could occur, but these should be rare in the absence of prior exposure to mouse protein. These will be an indication to stop therapy in the affected subjects, but accrual of new subjects may continue. An adverse event is any physical or clinical change or disease experienced by the subject from the date of randomization and up to two years from randomization for subjects continuing in the follow up period, whether or not considered related to the use of the investigational drug. This includes the onset of new illness and the exacerbation of the preexisting condition. For subjects who withdraw treatment during the treatment period, adverse events should be recorded through 28 days after the last administration of study treatment (OBI-822/OBI-821 or Control).

“Antibody” (Ab), “Antibodies” (Abs) and “immunoglobulins” (Igs) are glycoproteins having the same structural characteristics. While antibodies exhibit binding specificity to a specific antigen, immunoglobulins include both antibodies and other antibody-like molecules which generally lack antigen specificity. Polypeptides of the latter kind are, for example, produced at low levels by the lymph system and at increased levels by myelomas. The terms “antibody” and “immunoglobulin” are used interchangeably in the broadest sense and include monoclonal antibodies (e.g., full length or intact monoclonal antibodies), polyclonal antibodies, monovalent antibodies, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

“Variable” and “Complementarity Determining Regions” (CDRs)

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment which contains a complete antigen recognition and antigen binding site. In a two-chain Fv species, this region consists of a dimer of one heavy- and one light-chain variable domain in tight, non-covalent association. In a single-chain Fv species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequences of the constant domains of their heavy chains, antibodies (immunoglobulins) can be assigned to different classes. There are five major classes of immunoglobulins: IgA, IgD, IgE, IgG and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al. Cellular and Mol. Immunology, 4th ed. (2000). An antibody may be part of a larger fusion molecule, formed by covalent or non-covalent association of the antibody with one or more other proteins or peptides.

As used herein, “isolated antibody” can include an “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or non-proteinaceous solutes. In one embodiment, the antibody will be purified (1) to greater than 95% by weight of antibody as determined by, for example, the Lowry method, and in some embodiments more than 99% by weight, (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

The term “monoclonal antibody” (mAb) as used herein refers to an antibody obtained from a population of substantially homogeneous antibodies, e.g., the individual antibodies comprising the population are identical except for possible naturally occurring mutations that may be present in minor amounts. Thus, the modifier “monoclonal” indicates the character of the antibody as not being a mixture of discrete antibodies. Such monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones or recombinant DNA clones. It should be understood that the selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention.

In contrast to polyclonal antibody preparations which typically include different antibodies directed against different determinants (epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al., Nature, 256: 495 (1975); Harlow et al., Antibodies: A Laboratory Manual, (Cold Spring Harbor Laboratory Press, 2nd ed. 1988); Hammerling et al., in: Monoclonal Antibodies and T-Cell hybridomas 563-681 (Elsevier, N.Y., 1981)), recombinant DNA methods (see, e.g., U.S. Pat. No. 4,816,567), phage display technologies (See, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO98/24893; WO96/34096; WO96/33735; WO91/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; 5,661,016; Marks et al., Bio. Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996) and Lonberg and Huszar, Intern. Rev. Immunol. 13: 65-93 (1995).

“human monoclonal antibody” (HuMAb): A “human monoclonal antibody” is a mAb which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues.

“humanized antibody”: Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region of the recipient are replaced by residues from a hypervariable region of a non-human species (donor antibody) such as mouse, rat, rabbit or nonhuman primate having the desired specificity, affinity, and/or capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

“chimeric antibody” The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“antigen-binding portion” ‘or “antibody fragment”: “Antibody fragments” comprise only a portion of an intact antibody, wherein the portion retains at least one, and as many as most or all, of the functions normally associated with that portion when present in an intact antibody. In one embodiment, an antibody fragment comprises an antigen binding site of the intact antibody and thus retains the ability to bind antigen.

In another embodiment, an antibody fragment, for example one that comprises the Fc region, retains at least one of the biological functions normally associated with the Fc region when present in an intact antibody, such as FcRn binding, antibody half-life modulation, ADCC function and complement binding. In one embodiment, an antibody fragment is a monovalent antibody that has an in vivo half-life substantially similar to an intact antibody. For example, such an antibody fragment may comprise an antigen binding arm linked to an Fc sequence capable of conferring in vivo stability to the fragment.

The terms “cancer” and “cancerous” refer to or describe the physiological condition in mammals that is typically characterized by unregulated cell growth/proliferation. Examples of cancer include, but are NOT limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer. “Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

“Immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

“Immunoregulator” refers to a substance, an agent, a signaling pathway or a component thereof that regulates an immune response. “Regulating,” “modifying” or “modulating” an immune response refers to any alteration in a cell of the immune system or in the activity of such cell. Such regulation includes stimulation or suppression of the immune system which may be manifested by an increase or decrease in the number of various cell types, an increase or decrease in the activity of these cells, or any other changes which can occur within the immune system. Both inhibitory and stimulatory immunoregulators have been identified, some of which may have enhanced function in the cancer microenvironment.

“Immunotherapy” refers to the treatment of a subject afflicted with, or at risk of contracting or suffering a recurrence of, a disease by a method comprising inducing, enhancing, suppressing or otherwise modifying an immune response.

“Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication, condition or biochemical indicia associated with a disease.

“Potentiating an endogenous immune response” means increasing the effectiveness or potency of an existing immune response in a subject. This increase in effectiveness and potency may be achieved, for example, by overcoming mechanisms that suppress the endogenous host immune response or by stimulating mechanisms that enhance the endogenous host immune response.

“Subject” includes any human or nonhuman animal.

“Therapeutically effective amount” or “therapeutically effective dosage” of a drug or therapeutic agent, such as an Ab of the invention, is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

“Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

“Immune-related” response pattern refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents may require long-term monitoring of the effects of these agents on the target disease.

A therapeutically effective amount of a drug includes a “prophylactically effective amount,” which is any amount of the drug that, when administered alone or in combination with an anti-neoplastic agent to a subject at risk of developing a cancer (e.g., a subject having a pre-malignant condition) or of suffering a recurrence of cancer, inhibits the development or recurrence of the cancer. In preferred embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

“Tumor-infiltrating inflammatory cell” is any type of cell that typically participates in an inflammatory response in a subject and which infiltrates tumor tissue. Such cells include tumor-infiltrating lymphocytes (TILs), macrophages, monocytes, eosinophils, histiocytes and dendritic cells.

Immunogenic agent and Antibodies generated of the present invention may be constituted in a composition, e.g., a pharmaceutical composition, containing one Ab or a combination of Abs, or an antigen-binding portion(s) thereof, and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). A pharmaceutical composition of the invention may include one or more pharmaceutically acceptable salts, anti-oxidant, aqueous and nonaqueous carriers, and/or adjuvants such as preservatives, wetting agents, emulsifying agents and dispersing agents.

Preferred subjects include human patients in need of enhancement of an immune response. The immunotherapeutic methods disclosed herein are particularly suitable for treating human patients having a disorder that can be treated by potentiating an immune response. In certain embodiments, the methods are employed for treatment of subjects afflicted with a disease caused by an infectious agent. In preferred embodiments, the methods are employed for treatment of subjects afflicted with, or at risk of being afflicted with, a cancer.

“Cancer Immunotherapy”—as used herein, cancer immunotherapy can include, but not limited to, immune-based therapies capable of reducing tumor size in patients with metastatic cancer. Currently, there are three main approaches to cancer immunotherapy, a non-specific stimulation of immune reactions by stimulating effector cells and/or inhibiting regulatory cells, an active immunization to enhance specific anti-tumor reactions, known as cancer vaccines, and a passive transfer of anti-tumor antibodies or activated immune cells with antitumor activity, also known as adoptive immunotherapy (DeVita et al., 2008).

“Combination therapy”—in certain embodiments, the immunomodulatory agents discussed herein may be used in combination with one or more anti-proliferative/chemotherapeutic agent that are effective for reducing tumor burden without significant systemic toxicity and may act to improve the effectiveness of the immune response. The agents can be combined as co-administration combination therapy and/or co-formulated combination therapy.

Combination therapy in which two or more drugs are used together in some dosing regimen or administration form, typically has one or more goals of: (i) reducing the frequency at which acquired resistance arises by combining drugs with minimal cross-resistance, (ii) lowering the doses of drugs with non-overlapping toxicity and similar therapeutic profile so as to achieve efficacy with fewer side effects, i.e., increase therapeutic index, (iii) sensitizing cells to the action of one drug through use of another drug, such as altering cell-cycle stage or growth properties, and (iv) achieving enhanced potency by exploiting additivity, or greater than additivity, effects in the biological activity of two drugs (Pegram, M., et al (1999) Oncogene 18:2241-2251; Konecny, G., et al (2001) Breast Cancer Res. and Treatment 67:223-233; Pegram, M., et al (2004) J. of the Nat. Cancer Inst. 96(10):739-749; Fitzgerald et al (2006) Nature Chem. Biol. 2(9):458-466; Borisy et al (2003) Proc. Natl. Acad. Sci. 100(13):7977-7982). Loewe additivity (Chou, T. C. and Talalay, P. (1977) J. Biol. Chem. 252:6438-6442; Chou, T. C. and Talalay, P. (1984) Adv. Enzyme Regul. 22:27-55; Berenbaum, M. C. (1989) Pharmacol. Rev. 41:93-141) and Bliss independence/synergy (Bliss, C. I. (1956) Bacteriol. Rev. 20:243-258; Greco et al (1995) Pharmacol. Rev. 47:331-385) are methods used for calculating the expected dose-response relationship for combination therapy compared to monotherapy based on parameters such as IC50, the dose of drug needed to achieve 50% target inhibition and equal to Ki in the simplest case.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors.

Chemotherapeutic agents include compounds used in “targeted therapy” and conventional chemotherapy. Examples of chemotherapeutic agents include: erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine,dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo[4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethyl-ethanamine, NOLVADEX®, ISTUBAL®, VALODEX®), and doxorubicin (ADRIAMYCIN®), Akti-1/2, HPPD, and rapamycin.

More examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (MEK inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™ SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, Il), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, calicheamicin gamma1I, calicheamicin omegaI1 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (T-2 toxin, verracurin A, roridin A and anguidine); urethan; vindesine; dacarbazine; mannomustine; mitobronitol; mitolactol; pipobroman; gacytosine; arabinoside (Ara-C); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); icotininib; osimertinib; afatinib; dacomitinib; rociletinib; olmutinib; almonertinib; alflutinib; AC0010; BPI-7711; tarloxitinib; TAK-788; EAI045; BLU-945; nazartinib; naquotinib; mavelertinib; poziotinib; DBPR112; retinoids such as retinoic acid; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG™, 2C4, Genentech), trastuzumab (HERCEPTIN®, Genentech), tositumomab (Bexxar, Corixia), and the antibody drug-conjugate, gemtuzumab ozogamicin (MYLOTARG®, Wyeth).

Humanized monoclonal antibodies with therapeutic potential as chemotherapeutic agents in combination with the immunogenic/therapeutic agents of the present invention can include or exclude one or more of: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

“Standard-of-care therapeutic” is a treatment process, including a drug or combination of drugs, radiation therapy (RT), surgery or other medical intervention that is recognized by medical practitioners as appropriate, accepted, and/or widely used for a certain type of patient, disease or clinical circumstance. Standard-of-care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES) that provide detailed up-to-date information on the standard-of-care treatments for a wide variety of cancers (see NCCN GUIDELINES, 2013).

As used herein, additional aspects of the present disclosure includes aspects and factors relating to dose escalation, patient cohort, safety, and pharmacokinetics/pharmacodynamics analyses.

In broad terms, the immune system can be divided into innate and adaptive immunity. innate immunity, is the more primitive of the two and is comprised of non-specific defenses, such physical barriers (e.g., the skin), non-specific defensive cells (e.g., macrophages) and variety of cytokines (e.g., IL-1). In general, vaccines will not upregulate the innate system to a specific pathogen or disease, but adjuvants added to the vaccines may non-specifically activate innate immunity, which in turn may improve the adaptive immune response. Adaptive immune can be further divided into humoral (i.e., antibody) and cellular (e.g, cytotoxic T cells) immune responses. The effector cells of the humoral immune response are comprised of cells that specialize solely in adaptive immunity (e.g., T and B lymphocytes); however, cells of innate immunity provide essential functions (e.g., antigen presentation). Thus, for example, the induction of antibody production to a virus would require a series of complex interactions of several cell types. Simplified, these would include capture and processing of viral components (e.g., virus' envelope proteins) by dendritic cells, which would in turn be presented to T cells specific to the presented antigen. Once activated by presented antigen, the T cells would “help” virus-specific B cells to generate antibodies to the invading pathogen.

Tolerance

It has long been recognized that while the immune system has the capability to recognize host antigens, normally such responses are not observed (i.e., the immune system exhibits tolerance to self). This tolerance to self includes both “normal” as well as tumor antigens.

In one aspect, the current disclosure features a vaccine capable of disrupting the immune system's tolerance to the tumor antigen Globo H.

Tolerance can result from either central and/or peripheral tolerance. Central tolerance prevents maturation of T and B lymphocytes which recognize self. Self-tolerance is not absolute, and some B cells producing anti-self antibodies may be found in normal individuals. However, because of a lack of anti-self T-cell help to self-antigens—an essential component of B cell activation—antibodies to self are rarely found. Peripheral tolerance is the ongoing active suppression of the immune response to self, and is thought to be primarily maintained by Treg cells. Treg's are thought to prevent the induction of T cell help to self antigens, which include both normal and tumor antigens.

Increased antibody titer: In some embodiments the compositions and methods of this invention features a clinical benefit by producing an antibody response above a threshold titer. Below the threshold titer, the antitumor response may be insufficient to produce a meaningful clinical benefit.

Increased affinity of anti-Globo H antibodies: In some embodiments the methods of this disclosure feature administering the Globo H conjugate to the patient two, three, four, five, six, seven, eight, nine, or ten or more times.

Expansion of IgG subclasses: T cell help is able to induce B cells to switch their expression of heavy chain class and sub-classes. In humans, there are four IgG subclasses IgG1, IgG2, IgG3 and IgG4. Each IgG subclass has biological effector function that differentiates it from the other subclasses. The expression of all four subclasses may maximize the tumor-killing activity of the anti-Globo H response.

OBI-833 (Globo H-CRM197 Conjugate Vaccine) Immunological Profile

Recent studies have revealed that the expression of some glycans, such as Globo H and SSEA-3 and SSEA-4, was observed on breast cancer cells and breast cancer stem cells (BCSCs) (Chang et al. 2008b; Huang et al. 2013; Lou et al. 2014). All these findings support a rationale for the development of carbohydrate-based vaccines based on these cancer-specific glycans. Notably, the recent study indicated Globo H-CRM197 vaccine elicited more IgG antibodies, which are more selective for Globo H and the Globo series epitopes including SSEA-3 and SSEA-4, all of which were specifically overexpressed on breast cancer cells and breast cancer stem cells with SSEA-4 at the highest level (>90%) (Chang et al. 2008b).

Globo H has been evaluated as the target of active immunotherapy in a few clinical trials including the ongoing Phase II/Phase III clinical trial of OBI-822/OBI-821 under the US BB-IND 14,719 sponsored by OBI Pharma, Inc. The rationale for designing Globo-H conjugated to CRM-197 (a diphtheria toxin mutant) vaccine is based on the theory that tumor specific carbohydrate antigens can be recognized when processed and presented to a properly trained immune system. Immunization against these carbohydrate antigens results in a humoral antibody response. These antibodies are known to induce complement mediated cytotoxicity (CDC), inflammation, and phagocytosis of tumor cells by the reticuloendothelial system (opsonization). In addition to CDC, IgG antibodies of subclasses IgG1 and IgG3 in humans can also induce antibody dependent cell mediated cytotoxicity (ADCC). They are ideally suited for eradication of tumor cells and systemic metastases, which renders promises for OBI-833 to be used as a potential cancer vaccine.

OBI-833 is a glycoprotein conjugate comprised of a carbohydrate tumor antigen, Globo H, covalently linked to an inactive and nontoxic form of diphtheria toxin (DT) called cross-reacting material 197 (CRM197) as a carrier protein. It is intended to evoke an immune response against cancer cells by co-administering a natural cancer antigen, Globo H, conjugated to a potent immuno-stimulatory adjuvant, CRM-197. The detail of OBI-833 vaccine is as disclosed in PCT publication number: WO2014/107652.

OBI-821 is a saponin-based adjuvant derived from the bark of the Quillaja saponaria (QS) Molina tree. OBI-821 is structurally similar to QS-21 based on the comparison of physicochemical data. Both OBI-821 and QS-21 exist as mixtures of isomers. OBI-821 serves as an immunological adjuvant that could potentiate the humoral antibody response to OBI-833. The detail of OBI-821 adjuvant is as disclosed in PCT publication number: WO2019/191317.

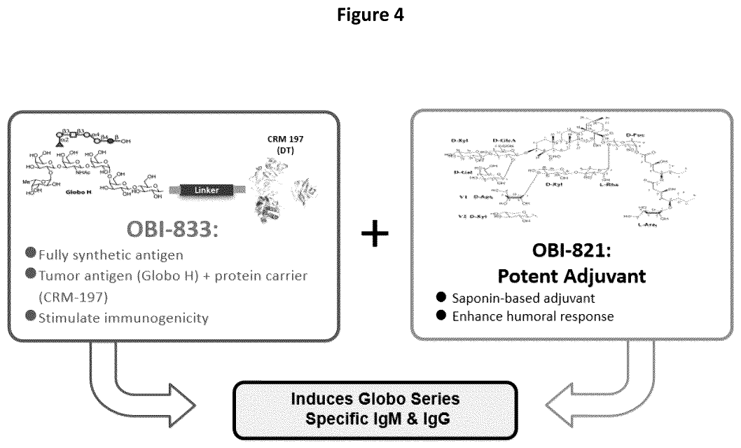

In order to characterize the immunogenic potential of the antigen/adjuvant combination, OBI-833/OBI-821 was tested in three pharmacology studies, including an immunogenicity study, an LL/2 tumor bearing mice model study, and a combination therapy study with gemcitabine. The structure of OBI-833 and OBI-821 is illustrated in FIG. 4.

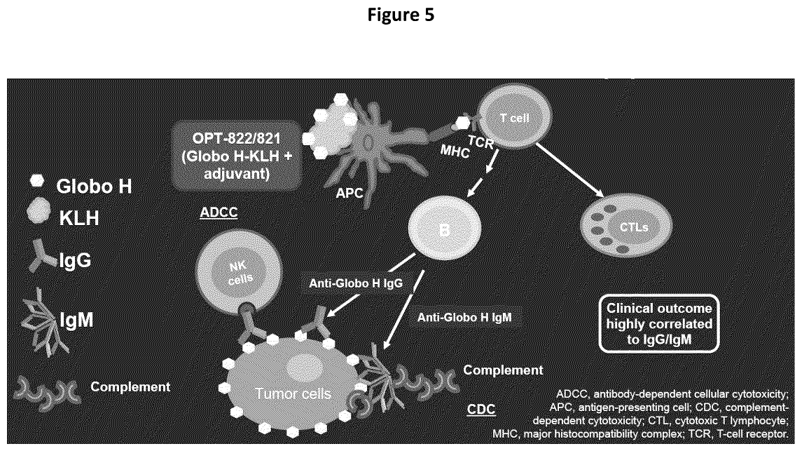

The pharmacology studies showed that active immunotherapy with OBI-833/OBI-821 can effectively stimulate anti-Globo H IgM and IgG responses in an in vivo murine model. Mouse vaccinated with OBI-833/OBI-821 can significantly inhibit tumor growth rate in an in vivo subcutaneous Globo H positive tumor implant model. It was also observed that the treatment of standard chemotherapeutic agent, gemcitabine, followed by vaccinated with OBI-833/OBI-821, did not affect the production of anti-Globo H IgM and IgG antibodies induced by OBI-833/OBI-821. The proposed OBI-833/OBI-821 Proof-of-Concept (POC) mechanism is illustrated in FIG. 5.

### EXAMPLES

Until Applicants' present disclosure and exemplary data in support of affirmative immunogenic response and therapeutic efficacy, there has been no prior conclusive demonstration/report of the effective use of the immunomodulatory agents as disclosed herein related to successful use of OBI-821 as an adjuvant as evidenced in the examples section, including Globo H Vaccine OBI-833 with OBI-821 in Cancer Clinical Trials.

Trial Overview: Globo H is a glycolipid found to be highly expressed in cancers. Active immunotherapy with OBI-833, a Globo H-CRM-197 conjugate, and OBI-821, an adjuvant in two phase trials, induced Globo H-specific antibodies which can mediate in vitro binding and cytotoxicity to Globo H-expressing cancer cells. The present disclosure provides demonstration of safety, tolerability, and immunogenicity of OBI-833/OBI-821 in subjects with advanced/metastatic gastric, lung, colorectal, or breast cancer. In addition, the disclosure also provides demonstration of immunogenicity, including humoral immune responses (anti-Globo H/anti-SSEA3/anti-SSEA4 IgG and IgM production) following subcutaneous administration of OBI-833/OBI-821 in subjects with non-small cell lung cancer.

Synopsis and Rationale for OBI-833 Phase 1 Clinical Trial Design (OBI-833-001)

Name of Compound: OBI-833 (Globo H-CRM197) and OBI-821 (Adjuvant) [OBI-833/OBI-821]101.161 Study Title: An Open-Label Study to Assess the Safety, Tolerability, and Efficacy of Active Immunotherapy with Dose Escalation and Cohort Expansion of OBI-833 (Globo H-CRM197) in Advanced/Metastatic Gastric, Lung, Colorectal, or Breast Cancer Subjects

Phase of Development: US and Taiwan-arm of multi-national Phase I dose escalation and cohort expansion study

Objectives:
(a) To evaluate the safety and tolerability of OBI-833/OBI-821 in subjects with advanced/metastatic gastric, lung, colorectal, or breast cancer
(b) To assess humoral immune responses (anti-Globo H IgG and IgM production) following subcutaneous administration of OBI-833/OBI-821
(c) To assess the clinical efficacy of OBI 833/OBI-821 (per RECIST 1.1 criteria)

Endpoints:
(a) Primary: Safety and Tolerability

All safety endpoints (toxicities and treatment-emergent adverse events [TEAEs], clinical laboratory evaluation, vital sign measurements, physical examination, and electrocardiogram [ECG]) were analyzed by descriptive statistics on the Safety Population. Changes from baseline were also summarized for clinical laboratory evaluation, vital sign measurements, physical examination, and ECG.
(b) Secondary: Immune response (anti-Globo H IgG and IgM production) and Tumor response

Immune Responses:

The Immune Response Population was used for all the analyses of immunological data. Concentrations of IgG and IgM antibodies to Globo H as determined by the glycan chip method at each assessment time point per dose cohort were summarized using descriptive statistics. Immune response for anti-Globo H IgG and IgM per subject at each time point were plotted by each dose cohort and cohort expansion phase.

Exploratory Immune Responses:

Concentrations of IgG and IgM antibodies to Globo H, SSEA-4, and SSEA-3 as determined by the glycan chip method at each assessment time point per dose cohort were summarized by cancer type as appropriate, or by Globo H IHC results at baseline (at Week 1). The maximal response, time to the maximal response, and the area under the response curve for each IgG type might be determined to aid the evaluation of a potential dose-response relationship. The results of exploratory analyses of other biomarkers (ADCC and CDC) at each assessment time point per dose cohort were summarized using descriptive statistics. Blood samples were collected at the time points to assess other immune responses or cellular immune responses by measuring the following antibodies and biomarkers:
Monitoring of immune response of anti-Globo H, anti-SSEA-3, and anti-SSEA-4 antibody production.
CTC (in selected Taiwan sites)
ADCC and CDC
T cell and B cell immune response (in selected Taiwan sites) [Cohort expansion phase only]
B/T cell immunogenomic analysis (in selected Taiwan sites)
Ex vivo immunogenicity analysis (in selected Taiwan sites)
Cobas epidermal growth factor receptor (EGFR) Mutation Tests (in selected Taiwan sites)

Secondary Tumor Response Endpoints

The Tumor Response Population was used for tumor response data. Response Evaluation Criteria in Solid Tumors (RECIST) 1.1 was used as the guideline for the analysis. Results of tumor responses were summarized at each assessment time point and the best tumor response during the entire study was presented per dose cohort using descriptive statistics.

Last survival status followed was summarized with frequency and percentage of total subjects in each category. Total duration of survival follow-up, defined as the interval (in months) between the date of first administration and the date of last known in the electronic case report form (eCRF) record were summarized using descriptive statistics. Overall survival was also estimated using Kaplan-Meier Method.

Clinical Efficacy Measurements

Full body CT scans (chest, abdomen, and pelvis) were performed at screening and used as the baseline scan. If full body CT scan had been performed within 4 weeks of the screening exam, then this previously performed scan could be used as the baseline scan. If the data of CT assessment were available within 6 weeks prior to the scheduled visit, the assessment of CT might not be performed based on the Investigator's discretion. Unscheduled tumor assessments could also be performed at the Investigator's discretion. Methodology used to assess tumors during the study period should be consistent with the methodology used at baseline/screening. For subjects where CT scans were contraindicated, MRIs could be performed instead. The tumor burden was categorized as either Measurable (Target Lesions) or Non-Measurable (Non-Target Lesions).

Survival and Subject Status

Unless the subject died, was lost to follow-up, or withdrew consent, all surviving subjects were followed up for survival status by phone contact or subjects' clinic visits, every 8 weeks for up to 48 weeks (dose escalation phase) or every 12 weeks for up to 24 weeks (cohort expansion phase), following the End of Study/Early Termination visit.

Design:

Open label, non-randomized dose escalation and cohort expansion trial.

Dose Groups and Treatments:

This study consists of dose escalation phase and cohort expansion phase. A standard 3+3 trial design will be used for OBI-833/OBI-821 dose escalation phase. The dosing of OBI-833 will be divided into 3 cohorts:
(a) Cohort 1: OBI-833 (10 μg Globo H/100 μg OBI-821)
(b) Cohort 2: OBI-833 (30 μg Globo H/100 μg OBI-821)
(c) Cohort 3: OBI-833 (100 μg Globo H/100 μg OBI-821)

Upon all of the dose escalation phase subjects completing 5 injections of OBI-833/OBI-821, the OBI Scientific Committee justified the safety, efficacy and immune response data of all the dose escalation phase subjects and recommend 30 μg of OBI-833 as the dose level for cohort expansion phase in lung cancer (Non-Small Cell Lung Cancer, NSCLC). Up to 14 NSCLC subjects will be enrolled in the cohort expansion phase.

Route of Administration: Subcutaneous (sc)

Dosing Frequency and Study Duration:

[Dose Escalation Phase]

Each subject in the dose escalation phase was given a total of 10 doses of OBI-833/OBI-821 subcutaneously at Weeks 1, 2, 3, 4, 6, 8, 12, 16, 20 and 24 (Visits 1, 2, 3, 4, 5, 6, 7, 8, 9 and 10, respectively).

Post treatment, subjects were continually evaluated for safety and immune response every 4 weeks until the end of the study, which is 12 weeks after the last dose (i.e., Week 36). Subsequently, subjects were followed for survival every 8 weeks up to 12 months after the end of the study.

[Cohort Expansion Phase]

In the cohort expansion phase, subjects were given OBI-833/OBI-821 at Weeks 1, 2, 3, 4, 6, 8, 12, 16, 20, 24, and every 8 weeks thereafter (Visits 1, 2, 3, 4, 5, 6, 7, 8, 9, 10, and every 8 weeks thereafter) until disease progression or up to 1 year after the last subject receives the first dose of study treatment. For the subjects who discontinued treatment because of disease progression, subjects were continually evaluated for safety and immune response every 8 weeks until the end of the study, which is 24 weeks after the last dose.

Subjects continued to be treated at discretion of the investigator after disease progression. The following clinical situations were conformed for continuous injection after disease progression (FDA Guidance for Industry, 2011):
(a) Subjects continued to meet all other study protocol eligibility criteria.
(b) All drug-related toxicities resolved to the baseline level.
(c) No deterioration of subject performance status.
(d) Did not delay imminent intervention to prevent serious complications of disease progression (e.g., CNS metastases).

For the subjects who were eligible and willing to continue injection after disease progression, subjects were treated with OBI-833/OBI-821 every 8 weeks for additional 3 injections, which is a total of 24 weeks after disease progression, to evaluate clinical and immune responses. Subsequently, subjects were followed up for survival after End of Study.

Dose Limiting Toxicity (DLT):

An event in the dose escalation phase means subjects were considered as having a dose limiting toxicity (DLT) if it occurred within the first 6 weeks after the administration of OBI-833/OBI-821 and met the following criteria:

Any Grade 3 or Grade 4 toxicities considered at least possibly related to the investigational drug.

Selection Criteria:

Inclusion Criteria:
(a) Subjects ≥21 years of age
(b) [Dose Escalation Phase]

Histologically or cytologically confirmed diagnosis of gastric, lung, colorectal or breast cancer on file

[Cohort Expansion Phase]

Histologically or cytologically confirmed diagnosis of Globo H-positive NSCLC
(c) [Dose Escalation Phase]

Subjects with recurrent or metastatic incurable disease that failed to respond to at least one line of anticancer standard therapy and for which standard treatment was no longer effective or tolerable.

[Cohort Expansion Phase]

Subjects with metastatic NSCLC who had achieved stable disease (SD), or partial response (PR) status after at least 1 regimen of anticancer therapy (i.e., chemotherapy, or targeted therapy, or PD-1/PD-L1 antagonists either alone or in combination), and there were no standard treatments available except permitted Target or PD-1/PD-L1 therapies.
(d) Measurable disease (i.e., present with at least one measurable lesion per RECIST, version 1.1 [Eisenhauer 2009]).
(e) [Dose Escalation Phase]

No known central nervous system (CNS) metastases or neurological symptoms possibly related to active CNS metastasis in Dose Escalation Phase.

[Cohort Expansion Phase]

Subjects with asymptomatic CNS metastases for at least four weeks before study drug treatment
(f) Performance status: ECOG 1
(g) Organ Function Requirements—Subjects must have had adequate organ functions as defined below:

Aspartate aminotransferase (AST)/alanine aminotransferase (ALT)≤3× upper limit of normal (ULN)
AST/ALT ≤3×ULN (upper limit of normal)
AST/ALT ≤5×ULN [with underlying liver metastasis]
Total bilirubin ≤2.0×ULN
Serum creatinine ≤1.5×ULNANC ≥1500/μL
Absolute neutrophil count (ANC)≥1500/μL
Platelets ≥100,000/μL
(h) Subjects of child-bearing potential must agree to use acceptable contraceptive methods during treatment and until the end of the study. Subjects not of childbearing potential (i.e., permanently sterilized, postmenopausal) could be included in the study. Postmenopausal was defined as 12 months with no menses without an alternative medical cause.
(i) Ability to understand and the willingness to sign a written informed consent document according to institutional guidelines.

Selection Criteria:

Exclusion Criteria:
(a) Patients who had not received standard chemotherapy, hormonal or targeted therapy for their underlying advanced/metastatic cancer.
(b) Subjects who were pregnant or breast-feeding at entry.
(c) Subjects with splenectomy.
(d) Subjects with known or clinically manifest, symptomatic CNS metastases in Dose Escalation Phase.
(e) Subjects with HIV infection, active hepatitis B infection or active hepatitis C infection.
(f) Subjects with any autoimmune or other disorders requiring iv/oral steroids or immunosuppressive or immunomodulatory therapies.
e.g., Type 1 juvenile onset diabetes mellitus, antibody positive for rheumatoid arthritis, Grave's disease, Hashimoto's thyroiditis, lupus, scleroderma, systemic vasculitis, hemolytic anemia, immune mediated thrombocytopenia, Crohn's disease, Ulcerative colitis or psoriasis etc.
(g) Subjects with any known uncontrolled inter-current illness including ongoing or active infections, symptomatic congestive heart failure (NYHA>2), unstable angina pectoris, cardiac arrhythmia, or psychiatric illness/social situations that would limit compliance with study requirements.
(h) [Dose Escalation Phase]

Subjects who had any of the following MEDICATIONS within 4 weeks prior to IP treatment:
Chemotherapeutic Agent
Immunotherapy [mAbs, Interferons, Cytokines (except GCSF)]
Immunosuppressants (e.g., cyclosporin, rapamycin, tacrolimus, rituximab, alemtuzumab, natalizumab, etc.).
IV/oral steroids except single prophylactic use in CT/MRI scan or other one-time use in approved indications. The interval between IV/oral steroids administration and first dose of OBI-833/OBI-821 must be more than pharmacological duration or 5 half-lives of administered steroids, whichever is longer. Uses of inhaled and topical steroids are allowed.
Another investigational drug

[Cohort Expansion Phase]

Subjects who had any of the following MEDICATIONS within 4 weeks prior to IP treatment:
Chemotherapeutic Agent
Immunotherapy [Interferons, Cytokines] (except PD-1/PD-L1 antagonists)
Immunosuppressants (e.g., cyclosporin, rapamycin, tacrolimus, rituximab, alemtuzumab, natalizumab, cyclophosphamide, etc.).
IV/oral steroids except single prophylactic use in CT/MRI scan or other one-time use in approved indications. The interval between IV/oral steroids administration and first dose of OBI-833/OBI-821 must have been more than pharmacological duration or 5 half-lives of administered steroids, whichever was longer. Uses of inhaled and topical (except on injection site) steroids were allowed.
Another investigational drug
(i) Subjects with pleural effusions and/or ascites, due to malignancy, requiring paracentesis every 2 weeks or more frequently.
(j) Subjects with any known severe allergies (e.g., anaphylaxis) to any active or inactive ingredients in the study drugs.

Study Evaluation and Assessment:

[Dose Escalation Phase]
(a) Tumor status confirmation: Full body CT scans (chest, abdomen, and pelvis) were performed at screening and then every 12 weeks until the end of the study.
(b) Safety and toxicity (adverse events) were evaluated at each visit, i.e., Weeks 1, 2, 3, 4, 6, 8, 12, 16, 20, 24, 28, 32, and 36 (End of Study).
(c) Hematology, serum chemistry and urine analysis were conducted at each visit, i.e., Weeks 1, 2, 3, 4, 6, 8, 12, 16, 20, 24, 28, 32, and 36.
(d) Immunology test for anti-Globo H IgG and IgM was conducted at Weeks 1, 3, 4, 6, 8, 12, 16, 20, 24, 28, 32, and 36.
(e) Tumor-specific antigen testing (CEA for all enrolled subjects) was performed prior to treatment and every 12 weeks until the end of the study.

[Cohort Expansion Phase]
(a) Tumor status confirmation: Full body CT scans (chest, abdomen, and pelvis) were performed at screening, Weeks 12, 24 and then every 8 weeks until disease progression or up to 1 year after the last subject received the first dose of study treatment. For the continuous injection subjects after disease progression, subjects performed tumor assessment every 8 weeks until the end of study.
(b) Safety and toxicity (adverse events) were evaluated at each visit until the end of the study.
(c) Hematology, serum chemistry and urine analysis was conducted at each visit until the end of the study.
(d) Immunology test for anti-Globo H IgG and IgM was conducted at Weeks 1, 3, 4, 6, 8, 12, 16, 20, 24, and every visit thereafter until the end of the study.
(e) Tumor-specific antigen testing (CEA and CYFRA 21-1 for all enrolled subjects in selected sites) was performed at screening, Weeks 12, 24, every 8 weeks thereafter until disease progression and end of study visit.

Exploratory Analysis

[Dose Escalation Phase]

Exploratory tests for the following biomarkers were conducted at Weeks 1, 3, 4, 6, 8, 12, 16, 20, 24, 28, 32, and 36:
(a) IHC for Globo H, SSEA-3, SSEA-4 and PD-L1 (Week 1; from paraffinated material of original surgery specimens)
(b) Monitoring of immune response of anti-Globo H, anti-SSEA-3, and anti-SSEA-4 antibody production.
(c) CTC (Week 1, 6 and 28 only)
(d) ADCC and CDC
(e) T cell and B cell responses (Weeks 12, 16, 20 and 24)

[Cohort Expansion Phase]

Exploratory tests for the following biomarkers were conducted at Screening, Weeks 1, 2, 3, 4, 6, 8, 12, 16, 20, 24, and every 8 weeks thereafter until end of study:
(a) IHC for Globo H, SSEA-3, SSEA-4, and PD-L1 (Screening; from fresh prepared (preferred) or paraffinated material of original surgery specimens)
(b) Monitored immune response of anti-Globo H, anti-SSEA-3, and anti-SSEA-4 antibody production.
(c) CTC (Week 1, 8, 64 and End of Study/Early Termination)
(d) ADCC and CDC
(e) T cell and B cell responses (at Weeks 8 or after until PTP1) (4 continue visits after either anti-Globo H, anti-SSEA-3, or anti-SSEA-4 IgG reaches 20 μg/ml in the serum)
(f) B/T cell immunogenomic analysis (Screening, Weeks 6, 12, 20, 64, and every 8 weeks until 8 weeks after 1st disease progression)
(g) Ex vivo Immunogenicity Analysis (Screening visit)
(h) Cobas EGFR Mutation Tests was conducted at Weeks 1, 12, 40 and post treatment period 1 and end of study for all subjects with known EGFR mutations in selected Taiwan sites. Cobas EGFR Mutation Tests were conducted at Early Termination visit for the early termination subjects other than PD.

Study Management:

[Dose Escalation Phase]
(a) After completing 5 injections of OBI-833/OBI-821 (i.e., at Visit 5, Week 6) in the first 3 or 6 subjects at the lower dose cohort in dose escalation phase, if there were no subjects of the first 3 or there were less than 2 subjects of the first 6 who develop DLT, enrollment could be started in the next higher dose cohort.
(b) Any subject who developed any DLT was discontinued from study treatment.
(c) All subjects who had a Grade 3 or 4 clinical or laboratory abnormality at the time of withdrawal from the study was followed until resolution to Grade 2 or less, unless it was unlikely to improve because of underlying disease.
(d) If there was more than 1 subject of the first 6 who develop DLT in dose escalation phase, then dose escalation was suspended until a full review by the Data and Safety Monitoring Board.

Recommendation for dose reduction resulted from the discussion of emerging safety data by the DSMB. If dose reduction was required for the subjects in Cohort 1 (10 μg cohort), the study was suspended temporally and could only be resumed to further enroll Cohort 1 subject without dose reduction if justified by recommendation of the DSMB upon analysis of emerging safety data. A Data and Safety Monitoring Board was established to assist the Sponsor in monitoring subject safety, risk/benefit, and decision for dose escalation, dose reduction and termination of the study.
(e) Patients who prematurely withdrew from dose escalation phase were replaced. To meet the replacement a subject fulfilled the following criteria:
   1) Not receives a total of 5 OBI-833/OBI-821 injections, during the dose escalation phase; and
   2) Reason of not completing 5 injections was other than development of related adverse event or a DLT (e.g. noncompliance or withdrew of consent).
(f) OBI Scientific Committee justified safety, efficacy and immune response data of all the dose escalation phase subjects and recommend 30 μg of OBI-833 as the dose level for cohort expansion phase in lung cancer (NSCLC).

[Cohort Expansion Phase]
(a) Patients were treated until disease progression, intolerable toxicity, withdrawal of consent, or up to 1 year after the last subject received the first dose of study treatment. Patients then entered the post-treatment period and follow-up phase.
(b) Subjects may have continued for treatment at discretion of the investigator after disease progression. The following clinical situations were conformed for continuous injection after disease progression:

Subjects continue to meet all other study protocol eligibility criteria
All drug-related toxicities resolved to the baseline level
No deterioration of subject performance status

Did not delay imminent intervention to prevent serious complications of disease progression (e.g., CNS metastases)

For the subjects who discontinued treatment because of disease progression, subjects were continually evaluated for safety and immune response every 8 weeks until end of the study, which was 24 weeks after the last dose. For the subjects who were eligible and willing continuous injection after disease progression, subjects were treated with OBI-833/OBI-821 every 8 weeks for additional 3 injections, which was a total of 24 weeks after disease progression, to evaluate clinical and immune responses. Subsequently, subjects were followed up for survival every 12 weeks up to 24 weeks after end of study.

Toxicology

The safety of OBI-833/OBI-821 vaccine was initially evaluated in a non-GLP 4-week repeat-dose rat toxicology study, in which OBI-833/OBI-821 at 2 doses (equivalent to 30 μg or 90 μg of Globo H) were administered once weekly via subcutaneous injection to Sprague Dawley rats for 4 weeks. OBI-833/OBI-821 was well tolerated without observation of adverse effects.

An additional toxicology study of OBI-833/OBI-821, a GLP 17-week repeat-dose rat toxicology study with an interim analysis at 11 weeks, was conducted to evaluate 10 subcutaneous injections in cancer patients in the planned Phase I clinical trial. The potential toxicity of the test article, OBI-833, and adjuvant, OBI-821, was assessed for separate administration or as a co-formulation via subcutaneous injection once weekly to Sprague Dawley rats for 17 weeks (Dosing Phase). In addition, reversibility, persistence, or delayed occurrence of any effects were assessed in a 4-week recovery period after the Dosing Phase. The doses of OBI-833/OBI-821 in the toxicology study (equivalent to 30 μg and 100 μg Globo H, and 100 μg of OBI-821) were selected based on the planned clinical dose range of Phase I study. The only observation caused by OBI-833 administration in combination with the adjuvant OBI-821 once weekly for 11 weeks in rats was minimal enhancement of the local irritation that the adjuvant produced at the injection sites. Injection site irritations were resolved clinically within a week and resolved microscopically within three weeks post-administration.

The toxicology studies demonstrated the safety and immunogenicity of the vaccine/adjuvant combination of OBI-833/OBI-821 and showed that the vaccine was well-tolerated at doses above that of the proposed clinical doses, on a per-kg body weight basis. All findings noted in the 4-week non-GLP and in the 11-week interim analysis of the 17-week GLP repeated dose toxicology studies in rats were not considered adverse and were likely due to inflammatory/immunostimulatory responses to the adjuvant, OBI-821.

Nonclinical Pharmacology

OBI-833 is a glycoprotein conjugate comprised of a carbohydrate tumor antigen, Globo H, covalently linked to an inactive and nontoxic form of diphtheria toxin (DT) called cross-reacting material 197 (CRM197) as a carrier protein. It is intended to evoke an immune response against cancer cells by co-administering a natural cancer antigen, Globo H, conjugated to a potent immuno-stimulatory adjuvant, CRM-197.

OBI-821 is a saponin-based adjuvant derived from the bark of the Quillaja saponaria (QS) Molina tree. OBI-821 is structurally similar to QS-21 based on the comparison of physicochemical data. Both OBI-821 and QS-21 exist as mixtures of isomers. OBI-821 serves as an immunological adjuvant that could potentiate the humoral antibody response to OBI-833.

In order to characterize the immunogenic potential of the antigen/adjuvant combination, OBI-833/OBI-821 was tested in three pharmacology studies, including an immunogenicity study, an LL/2 tumor bearing mice model study, and a combination therapy study with gemcitabine.

The pharmacology studies showed that active immunotherapy with OBI-833/OBI-821 can effectively stimulate anti-Globo H IgM and IgG responses in an in vivo murine model. Mouse vaccinated with OBI-833/OBI-821 can significantly inhibit tumor growth rate in an in vivo subcutaneous Globo H positive tumor implant model. It was also observed that the treatment of standard chemotherapeutic agent, gemcitabine, followed by vaccinated with OBI-833/OBI-821, did not affect the production of anti-Globo H IgM and IgG antibodies induced by OBI-833/OBI-821. Overall, these nonclinical pharmacology studies provided sound rationale to support clinical investigation of OBI-833/OBI-821.

Rationale for Trial Design

The primary purpose of the clinical trial was to investigate safety and tolerability of OBI-833/OBI-821 in subjects with advanced/metastatic incurable gastric, lung, colorectal, or breast cancer. Meanwhile, the humoral immune responses, i.e., anti-Globo H IgG and IgM production following administration of OBI-833/OBI-821 was assessed as the secondary objective of this study. Exploratory analysis of other immune responses such as Globo H, SSEA-3, SSEA-4, PD-L1 expression in tumor tissue samples, anti-Globo H, anti-SSEA-3, anti-SSEA-4 antibodies, CTC, ADCC, CDC were conducted as appropriate.

Upon the completion of 5 injections of OBI-833/OBI-821 in subjects in the dose escalation phase, the OBI Scientific Committee justified safety, efficacy and immune response data of all the dose escalation phase subjects and recommend 30 μg dose of OBI-833 and lung cancer (non-small cell lung cancer) for cohort expansion. Up to 14 lung cancers subjects were enrolled in the cohort expansion phase.

OBI-833/OBI-821 showed a delayed antibody response in the dose escalation phase of the OBI-833-001 study. In this situation, clinical progression may occur before the treatment has had sufficient time to be effective. Thus, continuous injection after disease progression was the potential approach to address this issue. Therefore, clinical progression that is asymptomatic and/or is not likely to result in life-threatening complications with further progression (e.g., new onset Central Nervous System (CNS) metastases) may allow the continuous administration of OBI-833/OBI-821 at the discretion of investigator and subjects should be fully informed for the subsequent treatment (FDA Guidance for Industry, Clinical Considerations for Therapeutic Cancer Vaccines, 2011).

Rationale for Starting Dose

The initial planned starting dose in this First in Humans clinical trial was 10 μg of OBI-833 (a Globo H equivalent) in combination with the adjuvant, 100 μg of OBI-821. Based on the reported NOAEL following 11 weekly SC injections of 100 μg of OBI-833 plus 100 μg of OBI-821, the highest dose evaluated in the 17 week toxicology study in rats, this dose of OBI-833/OBI-821 (100 μg/100 μg) was chosen as the highest anticipated human dose in the proposed Phase I clinical study. This proposed dosing plan was consistent with the FDA Guidance for developmental toxicity studies for preventive and therapeutic vaccines for infectious disease indications, and the proposed WHO 2013 Guidelines on the nonclinical evaluation of vaccine adjuvants and adjuvanted vaccines.

Eligibility Criteria

Inclusion Criteria
(a) Subjects ≥21 years of age
(b) [Dose Escalation Phase]

Histologically or cytologically confirmed diagnosis of gastric, lung, colorectal or breast cancer on file

[Cohort Expansion Phase]

Histologically or cytologically confirmed diagnosis of Globo H-positive NSCLC
(c) [Dose Escalation Phase]

Subjects with recurrent or metastatic incurable disease that failed to respond to at least one line of standard anticancer therapy and for which standard treatment is no longer effective or tolerable.

[Cohort Expansion Phase]

Subjects with metastatic NSCLC who had achieved stable disease (SD), or partial response (PR) status after at least 1 regimen of anticancer therapy (i.e., chemotherapy, or targeted therapy, or PD-1/PD-L1 antagonists either alone or in combination), and there are no standard treatments available except permitted Target or PD-1/PD-L1 therapies.
(d) Measurable disease (i.e., present with at least one measurable lesion per RECIST, version 1.1 [Eisenhauer 2009]).
(e) [Dose Escalation Phase]

No known central nervous system (CNS) metastases or neurological symptoms possibly related to active CNS metastasis in Dose Escalation Phase.

[Cohort Expansion Phase]

Subjects with asymptomatic CNS metastases for at least four weeks before study drug treatment
(f) Performance status: ECOG ≤1
(g) Organ Function Requirements—Subjects must have had adequate organ functions as defined below:
AST/ALT ≤3×ULN (upper limit of normal)
AST/ALT ≤5×ULN [with underlying liver metastasis]
Total bilirubin ≤2.0×ULN
Serum creatinine ≤1.5×ULN
ANC ≥1500/μL
Platelets >100,000/μL
(h) Subjects of child-bearing potential must agree to use acceptable contraceptive methods during treatment and until the end of the study. Subject not of childbearing potential (i.e., permanently sterilized, postmenopausal) could be included in the study. Postmenopausal was defined as 12 months with no menses without an alternative medical cause.
(i) Ability to understand and the willingness to sign a written informed consent document according to institutional guidelines.

Exclusion Criteria
(a) Patients who had not received standard chemotherapy, hormonal or targeted therapy for their underlying cancer.
(b) Subjects were pregnant or breast-feeding at entry.
(c) Subjects with splenectomy.
(d) Subjects with known or clinically manifest, symptomatic CNS metastases in Dose Escalation Phase.
(e) Subjects with HIV infection, active hepatitis B infection or active hepatitis C infection.
(f) Subjects with any autoimmune or other disorders requiring IV/oral steroids or immunosuppressive or immunomodulatory therapies.
e.g., Type 1 juvenile onset diabetes mellitus, antibody positive for rheumatoid arthritis, Grave's disease, Hashimoto's thyroiditis, lupus, scleroderma, systemic vasculitis, hemolytic anemia, immune mediated thrombocytopenia, Crohn's disease, Ulcerative colitis or psoriasis etc.
(g) Subjects with any known uncontrolled inter-current illness including ongoing or active infections, symptomatic congestive heart failure (NYHA>2), unstable angina pectoris, cardiac arrhythmia, or psychiatric illness/social situations that would limit compliance with study requirements.
(h) [Dose Escalation Phase]

Subjects with any of the following MEDICATIONS within 4 weeks prior to IP treatment:
Chemotherapeutic Agents
Immunotherapy [mAbs, Interferons, Cytokines (except GCSF)]
Immunosuppressants (e.g., cyclosporin, rapamycin, tacrolimus, rituximab, alemtuzumab, natalizumab, etc.).
IV/oral steroids except single prophylactic use in CT/MRI scan or other one-time use in approved indications. The interval between IV/oral steroids administration and first dose of OBI-833/OBI-821 must have been more than pharmacological duration or 5 half-lives of administered steroids whichever is the longer. Uses of inhaled and topical steroids were allowed.

Another investigational drug.

[Cohort Expansion Phase]

Subjects with any of the following MEDICATIONS within 4 weeks prior to IP treatment:
Chemotherapeutic Agents
Immunotherapy [Interferons, Cytokines] (except PD-1/PD-L1 antagonists)
Immunosuppressants (e.g., cyclosporin, rapamycin, tacrolimus, rituximab, alemtuzumab, natalizumab, cyclophosphamide, etc.).
IV/oral steroids except single prophylactic use in CT/MRI scan or other one-time use in approved indications. The interval between IV/oral steroids administration and first dose of OBI-833/OBI-821 must have been more than pharmacological duration or 5 half-lives of administered steroids whichever is longer. Uses of inhaled and topical steroids (except on injection sites) were allowed.

Another investigational drug.
(i) Subjects with pleural effusions and/or ascites, due to malignancy, requiring paracentesis every 2 weeks or more frequently.
(j) Subjects with any known severe allergies (e.g., anaphylaxis) to any active or inactive ingredients in the study drugs.

Treatment Plan

Subject Enrollment and Dose Escalation Procedure

This was a Phase I, open-label, non-randomized study with two phases: dose escalation phase and cohort expansion phase. Both phases evaluated safety and tolerability of OBI-833.

A standard 3+3 trial design were used for OBI-833/OBI-821 dose escalation phase. The dosing of OBI-833 will be divided into 3 cohorts:
Cohort 1: OBI-833 (10 μg Globo H/100 μg OBI-821)
Cohort 2: OBI-833 (30 μg Globo H/100 μg OBI-821)
Cohort 3: OBI-833 (100 μg Globo H/100 μg OBI-821)

In the dose escalation phase, the first three subjects of 10 μg dose cohort (Cohort 1) were enrolled sequentially with at least a 24-hour interval. If there is no DLT observed in any of these subjects, the trial would proceed to enroll subjects into the 30 μg dose cohort (Cohort 2). If one out of the first three Cohort 1 subjects develops a DLT, an additional three subjects would be further enrolled. If less than 2 of the 6 subjects (i.e. only 1 of the 6 subjects) develop DLT, dose escalation to 30 μg would be proceeded. If more than 1 of 6 subjects develop DLT, the study would be suspended temporally and could only be resumed to further enroll Cohort 1 subject without dose reduction justified by recommendation of the DSMB upon analysis of emerging safety data.

For 30 μg dose cohort (Cohort 2), firstly three patients were enrolled. If there was no DLT observed in any of these subjects, the trial would proceed to enroll subjects into the 100 μg dose cohort (Cohort 3). If one out of the first three Cohort 2 subjects develops a DLT, an additional three subjects would be further enrolled. If less than 2 of the 6 subjects (i.e. only 1 of the 6 subjects) develop DLT, dose escalation to 100 μg would be proceeded. If more than 1 of 6 subjects suffer from DLT, Data and Safety Monitoring Board would review the safety data to recommend if the study should be suspended or de-escalate to previous 10 μg cohort to complete a maximum of 6 subjects.

For 100 μg dose cohort (Cohort 3), firstly three subjects were enrolled in this cohorts. If one out of the first three Cohort 3 subjects develops a DLT, an additional three subjects would be further enrolled. If none of the first three or less than 2 of the 6 subjects experience DLT at the 100 μg dose level, then this dose will be maximum dose for escalation phase. If more than 1 of 6 subjects experience DLT at this dose level, study would be suspended temporarily and DSMB would review safety data to recommend if the study should be de-escalated to 30 μg to complete a maximum of 6 subjects.

Upon the completion of 5 injections of OBI-833/OBI-821 in subjects in the dose escalation phase, OBI Scientific Committee justified safety, efficacy and immune response data of all the dose escalation phase subjects and recommend 30 μg dose of OBI-833 for cohort expansion.

OBI Scientific Committee also examined the data of all the dose escalation phase subjects and preclinical efficacy data to evaluate the type of cancers to benefit from OBI-833/OBI-821 treatment. OBI Scientific Committee determined that lung cancer (Non-Small Cell Lung Cancer) subjects with SD or PR tumor status were to be enrolled for preliminary antitumor activity evaluation. Up to 14 subjects were to be enrolled in the cohort expansion phase.

The total number of subjects to be enrolled in the study was thus flexible: a maximum number of 18 subjects could be enrolled in the dose escalation phase; and a maximum number of 14 subjects could be enrolled in the cohort expansion phase. A maximum of total 32 subjects could be enrolled in this Phase I study.

Dose modification was considered, as justified by emerging safety data. Subject tumor biopsy/tissue samples was collected at Week 1 using fresh prepared or paraffinated material from original surgery specimens to test for expression level of Globo H, SSEA-3, SSEA-4 and PDL-1 for data analysis purpose. IHC data is not for eligibility criteria for Dose Escalation Phase but only Globo-H+ NSCLC subjects were enrolled in Cohort Expansion Phase.

Subjects who were eligible for participation in this study were enrolled into the dose cohort that was open at the time the subject was registered for enrollment in Dose Escalation Phase.

For each subject in dose escalation phase, maximum of 10 doses of OBI-833 (equivalent to 10, 30 or 100 μg of Globo H)/OBI-821 (100 μg) were administered subcutaneously at Weeks 1, 2, 3, 4, 6, 8, 12, 16, 20, and 24 (Visits 1, 2, 3, 4, 5, 6, 7, 8, 9 and 10, respectively). Patients who prematurely withdrew from dose escalation phase was replaced. To meet the replacement a subject should have fulfilled the following criteria: 1) Not have received a total of 5 OBI-833/OBI-821 injections during the dose escalation phase; and 2) Reason of not completing 5 injections was other than development of related adverse event or a DLT (e.g., noncompliance or withdrew of consent).

For Cohort Expansion Phase, patients were treated subcutaneously at Weeks 1, 2, 3, 4, 6, 8, 12, 16, 20, 24 and every 8 weeks until disease progression, intolerable toxicity, withdrawal of consent, or up to 1 year after the last subject receives the first dose of study treatment. Subjects may have continued treatment after disease progression at discretion of the investigator. The following clinical situations were conformed for continued injection after disease progression (FDA Guidance for Industry, 2011):
Subjects continued to meet all other study protocol eligibility criteria.
All drug-related toxicities resolved to the baseline level.
No deterioration of subject performance status.
Did not delay imminent intervention to prevent serious complications of disease progression (e.g., CNS metastases).

For the subjects who discontinued treatment because of disease progression, subjects were continually evaluated for safety and immune response every 8 weeks until the end of the study, which was 24 weeks after the last dose. For the subjects who were eligible and willing to continue injection after disease progression, subjects were treated with OBI-833/OBI-821 every 8 weeks for additional 3 injections, which was a total of 24 weeks after disease progression, to evaluate clinical and immune responses. Subsequently, subjects were followed up for survival every 12 weeks up to 24 weeks after end of study.

Week 1 was defined as the visit with the first sc administration of investigational drug. Blood samples were collected for routine blood test (part of safety assessment) and evaluation of immune responses at various weeks as indicated in the Time and Events Schedule (Table 1 and 2). Following the last dose of study drug, subjects were evaluated for safety and immune response every 4 weeks for dose escalation phase or 8 weeks for cohort expansion phase until the end of the study (“End of Study”), which was 12 or 24 weeks after the last dose administration. For subjects eligible for continued injection after disease progression were fully informed of the foreseeable risks or discomforts and other alternative treatment options.

OBI-833/OBI-821 Administration Schedule and Procedure

For each dose administration, a vial of OBI-833 drug product and a vial of OBI-821 drug product was used. The dosing solution was prepared by mixing the two components prior to injection. The mixing instruction for each dose level of OBI-833/OBI-821 is shown in example 1.
(a) For dose escalation phase, enrolled subjects were treated for a maximum of 10 doses of OBI-833/OBI-821 (sc) at Weeks 1, 2, 3, 4, 6, 8, 12, 16, 20, and 24 (Visit 1 to Visit 10). For Cohort Expansion Phase, subjects were treated until disease progression.
(b) A dose of 10 μg OBI-833/100 μg OBI-821, 30 μg OBI-833/100 μg OBI-821 or 100 μg OBI-833/100 μg OBI-821 mixture was injected subcutaneously according to respective cohorts 1, 2, and 3 in escalation phase and 30 μg OBI-833/100 μg OBI-821 in cohort expansion phase.
(c) Administration of OBI-833/OBI-821 combination should be completed within 2 hours after mixing.
(d) Subcutaneous injection (sc) over the arm (left or right) is preferable.
(e) If axillary lymph nodes were removed, avoid injections on the same arm.
(f) If axillary lymph nodes on both sides (left and right) were removed, injections can be made on the thigh (left or right).
(g) If local reaction occurs, rotate or alternate the injection site around the arms and thighs to minimize injection site irritation.

Study Procedures

Screening Phase—Prior to First Dosing of the Investigational Drug (Screening Visit)

A signed Informed Consent Form was properly obtained following a full explanation of the study protocol and prior to conducting any study related procedures.

For Dose Escalation Phase, the screening/baseline evaluations were to be conducted within 28 days prior to first dosing of the investigational drug. All entry/eligibility assessment must have been performed prior to first dosing of the investigational drug.
(a) Record demographics.
(b) Obtained significant medical history, other underlying disease/condition and history along the previous 12 months.
(c) Confirmed eligibility criteria (meeting all inclusion criteria, not having any exclusion criteria).
(d) Performed physical examination including vital signs (blood pressure, respiratory rate, pulse, and body temperature), height, weight, and ECOG performance status.
(e) Conducted urine pregnancy test. In case of positive results, the subject was not be eligible for the study.
(f) Conducted routine urinalysis—pH, protein, glucose, specific gravity, ketone, bilirubin, and urobilinogen. Analysis of urinary sediments (RBC, WBC, epithelial casts, hyaline casts, and bacterial) was optional.
(g) Conducted hematology test—included hemoglobin, hematocrit, WBC, RBC, platelet, differentials, RF and ESR.
(h) Conducted serum chemistry analysis—included sodium, potassium, chloride, calcium, magnesium, BUN, creatinine, ALT, AST, alkaline phosphatase, total protein, albumin, total bilirubin, LDH, amylase, lipase, cholesterol, triglycerides, cortisol, T3, T4, free T4, and TSH.
(i) Conducted HBV (e.g., HBsAg, HBeAg, and if clinically indicated, HBcAb and/or HBV DNA), HCV (e.g., anti-HCV) testing. Conducted HIV testing if no historical data was available. Optional tests (e.g., viral load) could have been ordered based on lab test results and Investigator's clinical judgment for suspected active infection on follow-up visits. Antiviral agents were allowed for the treatment and management of viral infections. However, interferon therapy was not allowed, and if indicated, the subject needed to be excluded from the study.
(j) Perform 12-Lead ECG.
(k) Perform Tumor Assessment:
Full body CT scans (chest, abdomen, and pelvis) were performed at screening and used as the baseline scan. If full body CT scan has been performed within 4 weeks before the screening exam (the date consent form sign by patient), then this previously performed scan could be used as the baseline scan.
To identify and record lesions to be evaluated for response based on RECIST 1.1 criteria.
Imaging was assessed by the study site radiologist or delegated investigators.
For cohort expansion phase, an interval of at least 6 weeks was required for Stable Disease (SD) status; an interval of at least 4 weeks was required for Partial Response (PR) status.
(l) Recorded use of concomitant medications.
(m) Performed re-testing of lab assessments: a re-test may have been allowed after 1 week (or by Investigators' judgment) from the initial lab test to re-confirm the subject's organ function or condition. However, the assessment needed to be conducted within the specified screening period prior to first dosing of the investigational drug.
(n) Performed pre-treatment tumor-specific antigen testing: CEA for all enrolled subjects.

For Cohort Expansion Phase, the screening/baseline evaluations were conducted within 35 days prior to first dosing of the investigational drug. All entry/eligibility assessment must have been performed prior to first dosing of the investigational drug.
(a) Obtained tumor biopsy/tissue samples of primary site (and distant metastases if possible) which was tested for Globo H, SSEA-3, SSEA-4 and PD-L1 expression by immunohistochemistry. Although fresh specimens were preferred, most subjects should have had historical tumor tissue specimens available, which could be submitted to the central lab for testing. If tumor tissue samples were not available, fresh tumor tissue samples were acquired. For Cohort Expansion Phase, fresh/archived tumor biopsies to test Globo H expression were essential.
(b) Record demographics.
(c) Obtained significant medical history, other underlying disease/condition and history along the previous 12 months.
(d) Confirmed eligibility criteria (meeting all inclusion criteria, not having any exclusion criteria).
(e) Performed physical examination including vital signs (blood pressure, respiratory rate, pulse, and body temperature), height, weight, and ECOG performance status.
(f) Conducted urine pregnancy test. In case of positive results, the subject will not be eligible for the study.
(g) Conducted routine urinalysis—pH, protein, glucose, specific gravity, ketone, bilirubin, and urobilinogen. Analysis of urinary sediments (RBC, WBC, epithelial casts, hyaline casts, and bacterial) is optional.
(h) Conducted hematology test—included hemoglobin, hematocrit, WBC, RBC, platelet, differentials, RF and ESR.
(i) Conducted serum chemistry analysis—included sodium, potassium, chloride, calcium, magnesium, BUN, creatinine, ALT, AST, alkaline phosphatase, total protein, albumin, total bilirubin, LDH, amylase, lipase, cholesterol, triglycerides, cortisol, T3, T4, free T4, and TSH.
(j) Conducted HBV (e.g., HBsAg, HBeAg, and if clinically indicated, HBcAb and/or HBV DNA), HCV (e.g., anti-HCV) testing. Conducted HIV testing if no historical data was available. Optional tests (e.g., viral load) could be ordered based on lab test results and Investigator's clinical judgment for suspected active infection on follow-up visits. Antiviral agents were allowed for the treatment and management of viral infections. However, interferon therapy was not allowed, and if indicated, the subject was excluded from the study.
(k) Perform 12-Lead ECG.
(l) Perform Tumor Assessment:

Full body CT scans (chest, abdomen, and pelvis) were performed at screening and used as the baseline scan. If full body CT scan had been performed within 4 weeks of the screening exam (the date consent form sign by patient), then this previously performed scan could be used as the baseline scan.

To identify and record lesions to be evaluated for response based on RECIST 1.1 criteria.

Imaging was assessed by the study site radiologist or delegated investigators.

For cohort expansion phase, an interval of at least 6 weeks was required for Stable Disease (SD) status; an interval of at least 4 weeks is required for Partial Response (PR) status.
(m) Record use of concomitant medications.
(n) Perform re-testing of lab assessments: a re-test may have been allowed after 1 week (or by Investigators' judgment) from the initial lab test to re-confirm the subject's organ function or condition. However, the assessment needed to be conducted within the specified screening period prior to first dosing of the investigational drug.
(o) Performed pre-treatment tumor-specific antigen testing: CEA and CYFRA21-1 for all enrolled subjects in selected sites.
(p) Collected blood samples for exploratory analysis of the B/T cell immunogenomic and Ex vivo immunogenicity analysis in selected Taiwan sites.

Treatment Period: Week 1—Visit 1

Eligible subject was treated with the first dose of the investigational drug according to the assigned cohort/dosage that was currently enrolling at the time the subject was eligible for enrollment.

For Cohort 1 in dose escalation phase, OBI-833 (equivalent to 10 μg Globo H)/OBI-821 (100 μg) was administered subcutaneously (sc). Subjects enrolled in Cohort 2 were administered OBI-833 (equivalent to 30 μg Globo H)/OBI-821 (100 μg) subcutaneously (sc); while subjects in Cohort 3 were administered OBI-833 (equivalent to 100 μg Globo H)/OBI-821 (100 μg) subcutaneously (sc). For cohort expansion, OBI-833 (equivalent to 30 μg Globo H)/OBI-821 (100 μg) was administered.

The following evaluations were conducted during Week 1 (visits as specified below):
(a) Performed physical examination including vital signs (blood pressure, respiratory rate, pulse, and body temperature) and weight prior to administration of investigational drug.
(b) Conducted routine urinalysis—pH, protein, glucose, specific gravity, ketone, bilirubin, and urobilinogen prior to administration of investigational drug. Analysis of urinary sediments (RBC, WBC, epithelial casts, hyaline casts, and bacterial) is optional.
(c) Conducted hematology test—includes hemoglobin, hematocrit, WBC, RBC, platelet, differentials, RF, and ESR prior to administration of investigational drug.
(d) Conducted serum chemistry analysis—included sodium, potassium, chloride, calcium, magnesium, BUN, creatinine, ALT, AST, alkaline phosphatase, total protein, albumin, total bilirubin, LDH, amylase, lipase, cholesterol, triglycerides, cortisol, T3, T4, free T4, and TSH, prior to administration of investigational drug.
(e) Collected blood samples for the measurement of anti-Globo H IgG and IgM titers prior to administration of investigational drug.
(f) For Dose Escalation Phase only: Obtained tumor biopsy/tissue samples of primary site (and distant metastases if possible) which was tested for Globo H, SSEA-3, SSEA-4 and PD-L1 expression by immunohistochemistry. Although fresh specimens were preferred, most subjects should have had historical tumor tissue specimens available, which could be submitted to the central lab for testing. If tumor tissue samples were not available, fresh tumor tissue samples should have been acquired prior to administration of investigational drug. However, in cases where tumor biopsies may not have been possible, subjects were not required to submit tissue samples.
(g) Collected blood samples for exploratory analysis of the following biomarkers prior to administration of investigational drug:

Monitored immune response of anti-Globo H, anti-SSEA-3 and anti-SSEA-4 antibody production.

CTC (only in selected Taiwan sites)

ADCC and CDC
(h) Administered investigational drug subcutaneously.
(i) Recorded use of concomitant medications.
(j) Monitored and recorded adverse events assessed by NCI CTCAE v4.0 post administration of investigational drug.
(k) Collected blood sample for the subjects with known EGFR mutations to be analyzed in the Cobas EGFR Mutation Tests in cohort expansion phase

Treatment Period Week 2 to Week 4 (Visit 2 to Visit 4)

Investigational drug was administered subcutaneously at Weeks 2, 3 and 4 (Visits 2, 3 and 4, respectively).

The following evaluations were conducted during Weeks 2 to 4 (Visit 2-Visit 4 as specified below):
(a) Performed physical examination including vital signs (blood pressure, respiratory rate, pulse, and body temperature) and weight—Weeks 2, 3 and 4 (Visits 2, 3 and 4) prior to administration of investigational drug.
(b) Conducted routine urinalysis—pH, protein, glucose, specific gravity, ketone, bilirubin, and urobilinogen prior to administration of investigational drug. Analysis of urinary sediments (RBC, WBC, epithelial casts, hyaline casts, and bacterial) was optional—Weeks 2, 3 and 4 (Visits 2, 3 and 4) prior to administration of investigational drug.
(c) Conducted hematology test—included hemoglobin, hematocrit, WBC, RBC, platelet, differentials, RF, and ESR—Weeks 2, 3 and 4 (Visits 2, 3 and 4) prior to administration of investigational drug.
(d) Conducted serum chemistry analysis—included sodium, potassium, chloride, calcium, magnesium, BUN, creatinine, ALT, AST, alkaline phosphatase, total protein, albumin, total bilirubin, LDH, amylase, lipase, cholesterol, triglycerides, cortisol, T3, T4, free T4, and TSH—Weeks 2, 3 and 4 (Visits 2, 3 and 4) prior to administration of investigational drug.
(e) Collected blood samples for the measurement of anti-Globo H IgG and IgM—Weeks 3 and 4 (Visits 3 and 4), prior to administration of investigational drug.
(f) Collected blood samples for exploratory analysis of the following biomarkers—Weeks 3 and 4 (Visits 3 and 4), prior to administration of investigational drug:

Monitored immune response of anti-Globo H, anti-SSEA-3 and anti-SSEA-4 antibody production.

ADCC and CDC
(g) Performed 12-Lead ECG—Week 4 (Visit 4) only for Dose Escalation Phase, prior to administration of investigational drug.
(h) Administered investigational drug subcutaneously—Weeks 2, 3 and 4 (Visits 2, 3 and 4).
(i) Recorded use of concomitant medications—Weeks 2, 3, and 4 (Visits 2, 3, and 4)
(j) Monitored and recorded adverse events assessed by NCI CTCAE v4.0—Weeks 2, 3, and 4 (Visits 2, 3, and 4).

Treatment Period: Week 6 to Week 24 (Visit 5 to Visit 10) or Disease Progression

[Dose Escalation Phase]

Investigational drug was administered subcutaneously at Weeks 6, 8, 12, 16, 20, and 24 (Visit 5 to Visit 10, respectively).

The following evaluations were conducted at these weeks (or visits):
(a) Performed physical examination including vital signs (blood pressure, respiratory rate, pulse, and body temperature) and weight—Weeks 6, 8, 12, 16, 20, and 24 (Visit 5-Visit 10); assessments were performed prior to administration of investigational drug.
(b) Conducted routine urinalysis—pH, protein, glucose, specific gravity, ketone, bilirubin, and urobilinogen. Analysis of urinary sediments (RBC, WBC, epithelial casts, hyaline casts, and bacterial) was optional—Weeks 6, 8, 12, 16, 20, and 24 (Visit 5-Visit 10); assessments were performed prior to administration of investigational drug.
(c) Conducted hematology test—included hemoglobin, hematocrit, WBC, RBC, platelet, differentials, RF, and ESR—Weeks 6, 8, 12, 16, 20, and 24 (Visit 5-Visit 10); assessments to be performed prior to administration of investigational drug.
(d) Conducted serum chemistry analysis—includes sodium, potassium, chloride, calcium, magnesium, BUN, creatinine, ALT, AST, alkaline phosphatase, total protein, albumin, total bilirubin, LDH, amylase, lipase, cholesterol, triglycerides, cortisol, T3, T4, free T4, and TSH—Week 6, 8, 12, 16, 20, and 24 (Visit 5-Visit 10); assessments were performed prior to administration of investigational drug. Collected blood samples for measurement of anti-Globo H IgG and IgM titer—Weeks 6, 8, 12, 16, 20, and 24 (Visit 5-Visit 10); samples were collected prior to administration of investigational drug.
(e) Collected blood samples for exploratory analysis for the following biomarkers—Weeks 6, 8, 12, 16, 20 and 24 (Visit 5-Visit 10) prior to administration of investigational drug: Monitored immune response of anti-Globo H, anti-SSEA-3 and anti-SSEA-4 antibody production.

ADCC and CDC
(f) Collected blood samples for exploratory analysis for the following biomarkers—Week 6 (Visit 5) prior to administration of investigational drug:

CTC (only in selected Taiwan sites)
(g) Collected blood sample for exploratory cellular immune response—Weeks 12, 16, 20 and 24 (Visit 7-Visit 10) prior to administration of investigational drug:

T cell and B cell immune response in selected Taiwan sites
(h) Performed tumor assessment according to the RECIST 1.1 criteria, including CT scan as required, every 12 weeks—Weeks 12 and 24 (Visits 7 and 10, respectively) prior to administration of investigational drug.
(i) Performed tumor-specific antigen testing (CEA for all enrolled subjects) every 12 weeks-Weeks 12 and 24 (Visits 7 and 10, respectively)
(j) Performed 12-Lead ECG at Weeks 12 and 20 (Visits 7 and 9, respectively) only, assessments were performed prior to administration of investigational drug.
(k) Administered investigational drug subcutaneously—Weeks 6, 8, 12, 16, 20, and 24 (Visit 5-Visit 10).
(l) Recorded use of concomitant medications—every Visit (Visit 5-Visit 10).
(m) Monitored and recorded adverse events assessed by NCI CTCAE v4.0—every Visit (Visit 5-Visit 10).

[Cohort Expansion Phase]

Investigational drug was administered subcutaneously at Weeks 6, 8, 12, 16, 20, 24, and every 8 weeks thereafter until disease progression.
(a) Performed physical examination including vital signs (blood pressure, respiratory rate, pulse, and body temperature) and weight—Weeks 6, 8, 12, 16, 20, 24 and every 8 weeks until disease progression; assessments were performed prior to administration of investigational drug.
(b) Conduct routine urinalysis—pH, protein, glucose, specific gravity, ketone, bilirubin, and urobilinogen. Analysis of urinary sediments (RBC, WBC, epithelial casts, hyaline casts, and bacterial) is optional—Weeks 6, 8, 12, 16, 20, 24 and every 8 weeks until disease progression; assessments were performed prior to administration of investigational drug.
(c) Conducted hematology test—included hemoglobin, hematocrit, WBC, RBC, platelet, differentials, RF, and ESR—Weeks 6, 8, 12, 16, 20, 24 and every 8 weeks until disease progression; assessments were performed prior to administration of investigational drug.
(d) Conducted serum chemistry analysis—includes sodium, potassium, chloride, calcium, magnesium, BUN, creatinine, ALT, AST, alkaline phosphatase, total protein, albumin, total bilirubin, LDH, amylase, lipase, cholesterol, triglycerides, cortisol, T3, T4, free T4, and TSH—Week 6, 8, 12, 16, 20, 24 and every 8 weeks until disease progression; assessments were performed prior to administration of investigational drug. Collected blood samples for measurement of anti-Globo H IgG and IgM titer—Weeks 6, 8, 12, 16, 20, 24 and every 8 weeks until disease progression; samples were collected prior to administration of investigational drug.
(e) Collected blood samples for exploratory analysis for the following biomarkers—Weeks 6, 8, 12, 16, 20 24 and every 8 weeks until disease progression prior to administration of investigational drug:

Monitored immune response of anti-Globo H, anti-SSEA-3 and anti-SSEA-4 antibody production.

ADCC and CDC
(f) Collected blood samples for exploratory analysis for the following biomarkers-Dose Escalation Phase: Week 6 (Visit 5) or Cohort Expansion Phase: Week 8, 64 (visits 6, 15) prior to administration of investigational drug:

CTC (only in selected Taiwan sites)
(g) Collected blood sample for exploratory cellular immune response only if either anti-Globo H, anti-SSEA-3, or anti-SSEA-4 serum concentration reaches 20 μeg/ml—4 continue visits prior to administration of investigational drug (Week 8 (Visits 6) or after):

T cell and B cell immune response in selected Taiwan sites
(h) Collected blood samples for exploratory analysis for the following biomarkers-Week 6, 12, 20, 64, and every 8 weeks until 8 weeks after the 1st disease progression (Visits 5, 7, 9, 15, and every 8 weeks until 8 weeks after the 1st disease progression) prior to administration of investigational drug:

B/T cell immunogenomic analysis in selected Taiwan sites
(i) Performed tumor assessment according to the RECIST 1.1 criteria, including CT scan as required, every 12 weeks-Weeks 12, 24 and every 8 weeks until disease progression prior to administration of investigational drug.
(j) Performed tumor-specific antigen testing (CEA and CYFRA21-1 for all enrolled subjects in selected sites) every 12 weeks-Weeks 12, 24 and every 8 weeks until disease progression
(k) Performed 12-Lead ECG at Weeks 12 and 8 weeks after final injection or disease progression, assessments were performed prior to administration of investigational drug.
(l) Administered investigational drug subcutaneously-Weeks 6, 8, 12, 16, 20, 24, and every 8 weeks until disease progression.
(m) Recorded use of concomitant medications-every visit until disease progression.
(n) Monitored and recorded adverse events assessed by NCI CTCAE v4.0-every visit until disease progression.
(o) Collected blood sample at weeks 12 and 40 for the subjects with known EGFR mutations to be analyzed in the Cobas EGFR Mutation Tests

Post Treatment Period to End of Study: Week 28 to Week 36 (Visit 11 to Visit 13) for Dose Escalation Phase; after disease progression for Cohort Expansion Phase

The following evaluations were conducted every 4 weeks for Dose Escalation Phase or every 8 weeks for Cohort Expansion Phase until the end of the study (“End of Study”), which is 12 weeks (Dose Escalation Phase) or 24 weeks (Cohort Expansion Phase) after the last subcutaneous dose of the investigational drug.
(a) Performed physical examination including vital signs (blood pressure, respiratory rate, pulse, and body temperature) and weight.
(b) Conducted routine urinalysis—pH, protein, glucose, specific gravity, ketone, bilirubin, and urobilinogen. Analysis of urinary sediments (RBC, WBC, epithelial casts, hyaline casts, and bacterial) was optional.
(c) Conducted hematology test—includes hemoglobin, hematocrit, WBC, RBC, platelet, differentials, RF and ESR.
(d) Conducted serum chemistry analysis—includes sodium, potassium, chloride, calcium, magnesium, BUN, creatinine, ALT, AST, alkaline phosphatase, total protein, albumin, total bilirubin, LDH, amylase, lipase, cholesterol, triglycerides, cortisol, T3, T4, free T4, and TSH.
(e) Collected blood samples for measurement of anti-Globo H IgG and IgM titer.
(f) Collected blood samples for exploratory analysis for the following biomarkers:

Monitored immune response of anti-Globo H, anti-SSEA-3 and anti-SSEA-4 antibody production.

ADCC and CDC
(g) Collected blood samples for exploratory analysis for the following biomarkers—Dose Escalation Phase: Week 28 (Visit 11) or Cohort Expansion Phase: End of study:

CTC (only in selected Taiwan sites)
(h) Collected blood sample for exploratory cellular immune response only if either anti-Globo H, anti-SSEA-3, or anti-SSEA-4 serum concentration reaches 20 μg/ml—4 continue visits prior to administration of investigational drug (8-Week after the last injection; PTP1):

T cell and B cell immune response in selected Taiwan sites
(i) Collected blood samples for exploratory analysis for the following biomarkers—Cohort Expansion Phase: 8 weeks after 1st disease progression:

B/T cell immunogenomic analysis (only in selected Taiwan sites)
(j) Performed tumor assessment according to the RECIST 1.1 criteria at weeks 36 (Visit 13) for Dose Escalation Phase or at every visit for the continuous injection subjects after disease progression in Cohort Expansion Phase.
(k) Performed tumor-specific antigen testing (CEA for all enrolled subjects) at weeks 36 (Visit 13) for Dose Escalation Phase or End of Study visit for Cohort Expansion Phase in selected sites (CEA and CYFRA21-1).
(l) Performed 12-lead ECG at the visits described in Table 1 and 2.
(m) Recorded uses of concomitant medications every visit.
(n) Monitored and recorded adverse events assessed by NCI CTCAE v4.0 every visit.
(o) Administered investigational drug subcutaneously every visit for the continuous injection subjects after disease progression in Cohort Expansion Phase
(p) Collected blood sample at post treatment period 1 and End of Study for the subjects with known EGFR mutations to be analyzed in the Cobas EGFR Mutation Tests in cohort expansion phase

Follow-Up Period (Up to 12 Months Following End of Study, i.e., Weeks 44 to 84)

Unless the subject met the off study criteria, all surviving subjects were followed up for survival status by phone contact or subjects' clinic visit, every 8 weeks for Dose Escalation Phase or 12 weeks for Cohort Expansion Phase for up to 48 weeks or 24 weeks, respectively, following the End of Study/Early Termination visit.

Early Termination

(Subjects, who prematurely terminated study treatment or evaluation for any reasons other than disease progression except continuous in post treatment period would only need to complete Early Termination Visit for the following evaluations before discontinuation from the study or continuing onto the follow-up phase. Disease progressive subjects who are not willing to continue into the post-treatment period will the perform EoS visit. If lab data was available within 1 week prior to EoS/EoT, lab tests could be waived. Surviving subjects would then enter follow-up period to follow up survival status by phone contact or subjects' clinic visit 12 weeks for Cohort Expansion Phase for up to 24 weeks.
(a) Performed physical examination and measured vital signs (blood pressure, respiratory rate, pulse, and body temperature), weight.
(b) Conducted routine urinalysis—pH, protein, glucose, specific gravity, ketone, bilirubin, and urobilinogen. Analysis of urinary sediments (RBC, WBC, epithelial casts, hyaline casts, and bacterial) was optional.
(c) Conducted hematology test—included hemoglobin, hematocrit, WBC, RBC, platelet, differentials, RF, and ESR.
(d) Conducted serum chemistry analysis—included sodium, potassium, chloride, calcium, magnesium, BUN, creatinine, ALT, AST, alkaline phosphatase, total protein, albumin, total bilirubin, LDH, amylase, lipase, cholesterol, triglycerides, cortisol, T3, T4, free T4, and TSH.
(e) Collected blood samples for measurement of anti-Globo H IgG and IgM titer.
(f) Collected blood samples for exploratory analysis for the following biomarkers:

Monitored immune response of anti-Globo H, anti-SSEA-3 and anti-SSEA-4 antibody production.

CTC (only in selected Taiwan sites)

ADCC and CDC
(g) Performed 12-Lead ECG.
(h) Performed Tumor assessment according to RECIST 1.1 criteria including CT scan as required.
(i) Performed tumor-specific antigen testing (CEA and CYFRA21-1 for all enrolled subjects in selected sites)
(j) Recorded adverse events assessed by NCI CTCAE v4.0.
(k) Recorded use of concomitant medications.
(l) Subjects, who discontinued the study treatment or evaluation (before End of Study) for reasons other than disease progression, were followed up for disease status by clinic visit, until Disease Progression. Same evaluations were conducted as those defined in the Post-Treatment Period.
(m) EGFR analyzed for the early termination subjects other than PD.

Other Study Procedures

Tumor tissue samples (and histology/pathology report, if possible) were collected at Week 1 prior to administration of the investigational drug and be submitted to a central laboratory to test the expression levels of Globo H, SSEA-3, SSEA-4, and PD-L1 by immunohistochemistry, as well as other tumor markers that may have been expressed in the lung, gastric, colorectal or breast tumors. Note that, subject tumor biopsy/tissue samples were collected to test the expression levels of Globo H, SSEA-3, SSEA-4, and/or PD-L1 were conducted at Week 1 for data analysis purpose and not for eligibility criteria in Dose Escalation Phase. Tumor biopsy/tissue samples were mandatory for Cohort Expansion subjects to test Globo H and other tumor markers such as SSEA-3, SSEA-4, and/or PD-L1 were for data analysis purposes. Globo H expression levels were collected and tested at screening visit for eligibility.

Blood samples were collected at various visits shown in Table 1 and 2 and stored with the intention of performing scientific research or immunological analysis on humoral and cellular mediated immune responses, and immunogenicity of the vaccine, using in-house glycan chip and Quantitative ELISA to detect immune response and monitor tumor response by biomarkers.

These tests included measurement of anti-Globo H IgG and IgM production. Blood samples were also collected for exploratory analysis of various biomarkers as follows:
(a) Monitored immune response of anti-Globo H, anti-SSEA-3 and anti-SSEA-4 antibody production.
(b) CTC (only in selected Taiwan sites)
(c) ADCC and CDC
(d) T cell and B cell immune response (in selected Taiwan sites)
(e) B/T cell immunogenomic analysis (in selected Taiwan sites)
(f) Ex vivo immunogenicity analysis (in selected Taiwan sites)
(g) Cobas EGFR Mutation Tests (in selected Taiwan sites)

Tissue and blood samples were analyzed using qualified assays by contracted laboratories under the supervision of OBI Pharma, Inc.

In order to evaluate the treatment response and the therapeutic mechanism of immunotherapies to specific tumor biology, blood and tumor samples were stored for present and relevant future studies in order to further explore the anti-tumor immune mechanism against specific tumor biology.

Future studies were carried out during and after the end of the clinical trial. This may be due to any trends or results that could potentially raise further questions and hypotheses leading to crucial findings and breakthroughs in cancer treatment.

Management of Toxicity and Treatment Discontinuation

General Management

Vital signs (blood pressure, respiratory rate, pulse and temperature) and inspection of injection sites were monitored at 0-5 minutes, at 15-30 minutes, and then at 2 hour (±30 minutes) after each injection of investigational drug.

For cohort in dose escalation phase, the first three subjects were hospitalized following first dose of the investigational drug injection, vital signs and inspection of injection sites were monitored after injection of investigational drug for following time point: at 0-5 minutes, at 15-30 minutes, at 2 hour (±30 minutes), at 4 hour (+30 minutes), at 6 hour (+30 minutes), before bed and before discharge.

Management of Drug-Induced Toxicities

Risks to the patients were mitigated with careful clinical monitoring and evaluation of laboratory safety parameters.

The most likely adverse effects that were anticipated in this study were local skin reaction at the injection site, fever, chills and sweats as a direct effect of active immunotherapy. These adverse effects seldom require therapy; but anti-pruritics may be used only if symptomatic. If necessary, NSAIDs may be used to control fever and pain, but steroids are prohibited. Pre-medication with anti-pruritics or steroids is prohibited. The study treatment continued despite these symptoms.

Slight to moderate dermal edema and erythema were observed in animal studies although these were not considered adverse effects. Investigators treated these conditions per their normal practice. Subjects were instructed to notify the Investigator if they experience drainage of fluid, injection-site reaction with break in skin and blue-black discoloration and swelling.

Less common but more severe allergic reactions include severe bronchospasm and anaphylaxis. In the presence of these conditions, treatment was immediately discontinued and the subject treated with epinephrine, steroids, oxygen, volume support, or other bronchodilators and supportive care as needed. The study treatment was discontinued and the subject was continuously monitored and may have been withdrawn from the study.

Autoimmune disorder may occur since low level of Globo H is expressed by epithelial cells. However, the likelihood of autoimmune disorder is rare since Globo H expression is confined to the apical epithelial cells at lumen borders, a site which appears not to be accessible to the immune system. Immune complex disease as manifested by skin, joint, renal or other changes could occur, but these should be rare in the absence of prior exposure to Globo H.

Treatment was discontinued, if there was evidence of frequent toxicities associated with severe epithelial cell injury. If any SAE's require the use of immunosuppressive therapies (e.g., cyclosporin, rapamycin, tacrolimus, rituximab, alemzutumab, natalizumab, iv/oral steroid, etc.) or immunomodulatory therapies (e.g., plasmapheresis, intravenous immunoglobulins), then the subject should be terminated from the treatment, and continue to be followed up until disease progression, early termination or end of study.

Definition and Management of Dose Limiting Toxicity

An event in dose escalation phase was considered a dose limiting toxicity (DLT) if it occurs within the first 6 weeks after the administration of OBI-833/OBI-821 and meets the following criteria:

Any Grade 3 or Grade 4 toxicities considered at least possibly related to the investigational drug.

Any subject who develops a DLT was terminated from study treatment.

All subjects who had a Grade 3 or 4 clinical or laboratory abnormality at the time of withdrawal from the study was followed until resolution to Grade 2 or less, unless it is unlikely to improve because of underlying disease.

If there was any subject of the first 3 or there is more than 1 subject of the first 6 who develop DLT, then the dose escalation was suspended until a full review by the Data and Safety Monitoring Board. Dose modification, such as a 50% dose reduction for the next dose cohort, may have been considered, as justified by emerging safety data. If dose reduction was required for the subjects in cohort 1 (10 μg Cohort), study was suspended temporally. Data and Safety Monitoring Board reviewed the emerging safety data and instructed if the study could be resumed and continue to enroll Cohort 1 subject without dose reduction.

Guidelines for Individual Subject Study Treatment (OBI-833/OBI-821) Discontinuation

In the event of a Grade 3 immune-associated AE or emergent SAE as listed below, the subject was required to discontinue study drug treatment. The Data and Safety Monitoring Board would have been notified of these events and safety reporting to the various health authorities would follow each country's respective health agency guidelines.
Auto-immune diseases included but not limited to severe Guillain-Barré Syndrome (GBS) or chronic inflammatory demyelinating polyneuropathy (CIDP).
Encephalopathy or any other significant CNS involvement.
Anaphylaxis or respiratory failure requiring intubation and/or ventilator support.
Indication for emergency intervention and life-threatening consequences such as malignant hypertension, shock leading to vital organ impairment, severe cardiac arrhythmias, etc.
Vasculitis leading to severe ischemia and vascular neuropathy.
Nephritis leading to severe renal impairment.
Hepatitis leading to severe hepatic dysfunction.
Adrenal insufficiency—life threatening adrenal crisis.
Severe pancreatitis.
Thyroiditis leading to ≥grade 4 hyperthyroidism or hypothyroidism.
Autoimmune hemolytic anemia.
And any other potential life threatening or disabling adverse events attributable to the study drug in the opinion of the Investigator and medical monitor.

Immune related AEs that would NOT be considered emergent or serious, and may NOT require study treatment discontinuation may include:
Inflammation that can be attributable to a local antitumor reaction at tumor sites or in draining lymph nodes. This includes inflammatory reactions at sites of tumor resections or exposed to radiation therapy.

If toxicity occurred regardless of investigational drug administration that does not meet study treatment discontinuation criteria:
The investigational drug administration may have been delayed for up to 2 consecutive weeks.
In the event that the administration of the investigational drug was delayed for more than 2 consecutive weeks, then the Investigator conferred with the Sponsor's Medical Monitor to discuss the appropriateness of continued treatment.

A Data and Safety Monitoring Board was established to assist Sponsor in monitoring the subject safety, risk/benefit, dose escalation, dose reduction and termination of the study.

Treatments Permitted and Prohibited During Study

Permitted Treatments During Study

[Dose Escalation Phase]
(a) Analgesics: Non-steroidal anti-inflammatory drugs (NSAID), analgesics other than NSAID (like aspirin), and opiates may be used for pain control.
(b) GCSF and Hematopoietic growth factors were allowed in the dose escalation phase if deemed necessary by physicians.
(c) Antihistamine (H1 and H2) and decongestant: cetirizine HCl, fexofenadine HCl, levocetirizine, and pseudoephedrine.
(d) Antiemetic and anti-pruritics therapy.
(e) Therapy with bisphosphonates (e.g., to treat bone metastases), was at the discretion of the investigator.

[Cohort Expansion Phase]
(a) Analgesics: Non-steroidal anti-inflammatory drugs (NSAID), analgesics other than NSAID (like aspirin), and opiates may have been used for pain control.
(b) Antihistamine (H1 and H2) and decongestant: diphenhydramine, hydroxyzine, cimetidine, ranitidine, famotidine, cetirizine HCl, fexofenadine HCl, levocetirizine, and pseudoephedrine.
(c) Antiemetic (e.g., ondansetron) and anti-pruritics (e.g., diphenhydramine or hydroxyzine) therapy.
(d) Therapy with bisphosphonates (e.g., to treat bone metastases), is at the discretion of the investigator.
(e) EGFR/ALK TKIs (e.g., gefitinib, erlotinib, afatinib and crizotinib), EGFR mAb (Necitumumab), bevacizumab and PD-1/PD-L1 inhibitors (e.g., pembrolizumab and nivolumab)

Prohibited Treatments During Study

The following treatments were prohibited during the Treatment and Post-treatment periods of the study until End of Study:

[Dose Escalation Phase]
(a) Anti-cancer treatment: no other anti-cancer therapy was allowed during the study. The list of other chemotherapy agents, chemotherapeutic agents, active or passive immunotherapy, included, but was not limited to:
   Chemotherapeutic agents: antimetabolites, alkylating agents, vinca alkaloid, epipodophyllotoxins, taxanes, camptothecins, antitumor antibiotics, nitrosoureas folate analogue metabolic inhibitor, and miscellaneous cytotoxic agents.

Surgery

Radiotherapy
(b) Biologic Agents: Monoclonal antibodies, Interferons and Interleukins
(c) Immunotherapy: cyclosporin, rapamycin, tacrolimus, rituximab, alemtuzumab, natalizumab etc.
(d) Steroids: iv/oral steroids except single prophylactic use in CT/MRI scan or other one-time use in approved indications. The interval between iv/oral steroids administration and first dose of OBI-833/OBI-821 must have been more than pharmacological duration or 5 half-lives of administered steroids whichever is the longer.

Uses of inhaled and topical steroids were allowed.
(e) Tyrosine kinase inhibitors
(f) Alternative and complementary medicine may affect immune system
(g) Other investigational drugs

[Cohort Expansion Phase]
(a) Anti-cancer treatment: The list of chemotherapy agents, active or passive immunotherapy included, but was not limited to:
   Chemotherapeutic agents: antimetabolites, alkylating agents, vinca alkaloid, epipodophyllotoxins, taxanes, camptothecins, antitumor antibiotics, nitrosoureas folate analogue metabolic inhibitor, and miscellaneous cytotoxic agents. EGFR/ALK TKIs are permitted.

Surgery

Radiotherapy
(b) Biologic Agents: Interferons, Interleukins, Denosumab (RANKL inhibitor), GCSF and Hematopoietic growth factors
(c) Immunotherapy: cyclosporin, rapamycin, tacrolimus, rituximab, alemtuzumab, natalizumab, cyclophosphamide, etc (except PD-1/PD-L1 antagonists).
(d) Steroids: iv/oral steroids except single prophylactic use in CT/MRI scan or other one-time use in approved indications. The interval between iv/oral steroids administration and first dose of OBI-833/OBI-821 must have been more than pharmacological duration or 5 half-lives of administered steroids whichever is the longer.

Topical (except injection sites) and inhaled steroid use was allowed
(e) Alternative and complementary medicine may affect immune system.
(f) Other investigational drugs
(g) Subjects who continuous treatment after disease progression were allowed to concomitant use of all approved anti-cancer therapies

Investigational Drug Information

OBI-833 (Globo H-CRM197)

Formulation:

OBI-833 is supplied as a sterile lyophilized powder drug product in single-use 2 mL amber borosilicate glass serum vials. Each vial contains 150 μg Globo H linked with CRM197 along with potassium phosphate buffered saline, Sucrose, and polysorbate 80. The physical appearance specification for the drug product is lyophilized cake/powder. The reconstituted solution of OBI-833 drug product is a clear liquid.

Source and Pharmacology:

OBI-833 is a glycoconjugate comprised of a carbohydrate tumor antigen, Globo H, which is covalently linked to the carrier protein, an inactive and nontoxic form of diphtheria toxin (DT) called cross-reacting material 197 (CRM197).

Immunization of mice with Globo H-CRM197 (DT) induced antibodies reactive with Globo H, SSEA-3, and SSEA-4, suggesting that a Globo H-based vaccine will target tumor cells expressing Globo H, SSEA-3, and SSEA-4. More specifically, as Globo H, SSEA-3, and SSEA-4 are found to be expressed in human tissues of breast cancer, hepatocellular cancer (HCC), lung cancer, oral cancer, gastric and pancreatic cancers, it is postulated that the anti-Globo H, SSEA-3 and SSEA-4 antibodies generated from immunization of OBI-833/OBI-821 can target tumor cells in aforementioned cancer types.

OBI-821

Formulation:

OBI-821 is lyophilized powder/cake in individual 2 mL amber borosilicate glass serum vial containing 125 μg OBI-821, sodium phosphate buffered saline, and Trehalose.

Source and Pharmacology:

OBI-821 is a saponin based adjuvant derived from the bark of the Quillaja saponaria Molina tree. OBI-821 is a purified saponin, which is structurally similar to descriptions found in the literature for another adjuvant, QS-21. OBI-821 exists as a mixture of isomers. The primary component is designated as OBI-821-ViA, with the balance being a group of closely related analogs.

OBI-821 has been shown to stimulate a variety of immunological activities including antigen-specific antibody to carbohydrate tumor antigen conjugated to a carrier protein. OBI-821 also augments the induction of major histocompatibility complex (MHC)-restricted class I cytotoxic T lymphocytes to subunit antigen vaccines, as well as antigen-specific cellular proliferation.

Clinical Trial Material (CTM) Supply, Packaging, Labeling and Storage

All CTM was supplied by OBI Pharma, and must remain under adequate security, storage condition. Do not use any CTM after the expiration date, which is labeled on the investigational drug container.

Investigational Drug Supply

OBI-833 (equivalent to 150 μg Globo-H) and OBI-821 (125 μg) are provided in separate single-use vials. OBI-821 is mixed with OBI-833 at the time of each injection (within 2 hours after mixing). Following the injections, the leftover OBI-833/OBI-821 mixtures are not recyclable.

Each injection dose consists of a mixture of 100 μg OBI-821 and OBI-833 containing 10 μg, 30 μg, or 100 μg of Globo H equivalents. OBI-833 drug product is reconstituted in water and mixed with reconstituted OBI-821 immediately. The freshly combined OBI-833/OBI-821 mixture is administered by subcutaneous injection. Example 1 is the detailed procedure for preparation and mixing of OBI-833 and OBI-821 for injection.

Investigational Drug Storage

OBI-833 and OBI-821 are provided in separate single-use vials. OBI-821 is to be mixed with OBI-833 at time of injections. The recommended storage temperature for both OBI-833 and OBI-821 drug product vials are between 2-8° C.

Study Endpoint

Primary Endpoints The primary endpoint is safety and tolerability of OBI-833/OBI-821 assessed by adverse events, changes in laboratory values, and changes in vital signs and physical exam results.

Secondary Endpoints
(a) Immune response as assessed by anti-Globo H IgG and IgM production.
(b) Tumor response per RECIST 1.1 criteria.

Safety Assessment
(a) Toxicity and adverse events were assessed by NCI CTCAE v4.0 following subcutaneous doses of OBI-833/OBI-821 immunization.
(b) Safety and toxicity was assessed according to the dose of OBI-833/OBI-821 the subjects received and on all subjects who received at least one dose of OBI-833/OBI-821. Every effort should be made to follow up all subjects and all scheduled evaluations should be performed until study closure or the death of the subjects irrespective of withdrawals from the treatments.
(c) Any clinically significant abnormalities persisting at the end of the study/early termination was followed by the investigator until resolution or until a clinically stable endpoint is reached.

Safety Variables
(a) Toxicity & adverse events (as assessed by NCI CTCAE v4.0).
(b) Safety laboratory examination (Urinalysis, hematology and serum chemistry).
(c) Vital signs (blood pressure, respiratory rate, pulse and temperature).
(d) Liver and renal functions.
(e) Physical exam.
(f) ECG.

Response Criteria

Criteria for Tumor Response by RECIST 1.1:

RECIST 1.1 was used as the guideline for the analysis of this study. Images were taken from the chest, abdomen and pelvis area. A Baseline/Screening tumor burden was obtained by evaluating the entire organ systems included in the body systems imaged. This tumor burden was categorized as either Measurable (Target Lesions) or Non-Measurable (Non-Target Lesions). The purpose of establishing this Baseline/Screening is to allow for subsequent assessment of on-treatment response.

Definitions for Measurability of Tumor Lesions

Measurable:

Tumor lesions must be accurately measured in at least one dimension (longest diameter in the plane of measurement is to be recorded) with a minimum size of:
(a) 10 mm by CT scan (CT scan slice thickness recommended to be in between 2.5 mm and 5 mm).
(b) 10 mm caliper measurement by clinical exam (lesions which cannot be accurately measured with calipers should be recorded as non-measurable).
(c) 20 mm by chest X-ray.
(d) Malignant lymph nodes:

To be considered pathologically enlarged and measurable, a lymph node must be <15 mm in short axis when assessed by CT scan (CT scan slice thickness recommended to be in between 2.5 mm and 5 mm). At baseline and during follow-up, only the short axis was measured and followed See also notes below on ‘Baseline documentation of target and non-target lesions’ for information on lymph node measurement. Tumor lesions situated in a previously irradiated area, or in an area subjected to other loco-regional therapy, are usually NOT considered measurable unless there has been demonstrated progression in the lesion.

Non-Measurable:

All other lesions, including small lesions (longest diameter <10 mm or pathological lymph nodes with ≥10 to <15 mm short axis) as well as truly non-measurable lesions.

Lesions considered truly non-measurable include: leptomeningeal disease, ascites, pleural or pericardial effusion, inflammatory breast disease, lymphangitic involvement of skin or lung, abdominal masses/abdominal organomegaly identified by physical exam that is not measurable by reproducible imaging techniques.

Recording Tumor Lesions

When more than 1 measurable lesion is present at baseline all lesions up to a maximum of 5 lesions total (and a maximum of 2 lesions per organ) representative of all involved organs should be identified as target lesions and were and measured at baseline (this means in instances where subjects have only 1 or 2 organ sites involved a maximum of 2 and 4 lesions respectively were recorded).

A sum of the diameters (longest for non-nodal lesions, short axis for nodal lesions) for all target lesions was calculated and reported as the baseline sum diameters. If lymph nodes are to be included in the sum, then as noted above, only the short axis is added into the sum. The baseline sum diameters were used as reference to further characterize any objective tumor regression in the measurable dimension of the disease.

All other lesions (or sites of disease) including pathological lymph nodes should be identified as non-target lesions and should also be recorded at baseline. Measurements are not required and these lesions should be followed as ‘present’, ‘absent’, or in rare cases ‘unequivocal progression’.

In addition, it is possible to record multiple non target lesions involving the same organ as a single item on the case record form (e.g., ‘multiple enlarged pelvic lymph nodes’ or ‘multiple liver metastases’).

Response Evaluation

Target Lesions
(a) Complete Response (CR): Disappearance of all target lesions. Any pathological lymph nodes (whether target or non-target) must have reduction in short axis to <10 mm.
(b) Partial Response (PR): At least a 30% decrease in the sum of diameters of target lesions, taking as reference the baseline sum diameters.
(c) Stable Disease (SD): Neither sufficient shrinkage to qualify for PR nor sufficient increase to qualify for PD, taking as reference the smallest sum-diameters while on study. Measurements must have met the SD criteria at least once after study entry at a minimum interval of 6 weeks.
(d) Progressive Disease (PD): At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression).
(e) At each time point, the presence or absence of new lesions was assessed.

Non-Target Lesions
(a) Complete Response (CR): Disappearance of all non-target lesions and normalization of tumor marker level. All lymph nodes must be non-pathological in size (<10 mm short axis).
(b) Stable Disease (SD): Persistence of one or more non-target lesion(s) and/or maintenance of tumor marker level above the normal limits.
(c) Progressive Disease (PD): Unequivocal progression of existing non-target lesions. (Note: the appearance of one or more new lesions is also considered progression). The overall response situations are listed in Table 1.

TABLE 1
Overall Response Table

| Target | Non-Target | New | Time Point |
|---|---|---|---|
| Lesions | Lesions | Lesions¹ | Response |
| CR | CR | No | CR |
| CR | SD | No | PR |
| CR | UE | No | PR² |
| PR | UE | No | PR² |
| PR | CR | No | PR |
| PR | SD | No | PR |
| SD | UE | No | SD |
| SD | CR | No | SD |
| SD | SD | No | SD |
| PD | ANY | Yes/No | PD |
| ANY | Unequivocal PD/PD | Yes/No | PD |
| ANY | ANY | Yes | PD |
| UE | Non-PD | No | UE |
| CR | NA⁴ | No | CR |
| PR | NA⁴ | No | PR |
| SD | NA⁴ | No | SD |
| NA³ | SD | No | SD |
| NA³ | CR | No | CR |
| NA³ | UE | No | UE |
| NA³ | NA⁴ | No | UE |

¹Identification of new lesions at a post-baseline time point will result in time point response of PD. If an identified new lesion subsequently becomes UE, the time point response was recorded as PD unless the new lesion has proven to have resolved.
²If a non-target lesion is classified as UE, a designation of PR may be assigned based on information from the target lesions.
³No target lesions identified at baseline.
⁴No non-target lesions identified at baseline.
⁵CR = complete response, PR = partial response, SD = stable disease, PD = progressive disease, UE = unable to evaluate, and NA = not applicable.

Criteria for Removal from Protocol Therapy and off Study Criteria

Criteria for Removal from Protocol Therapy

Subjects were discontinued from the study treatment with the investigational drug for any of the following reasons:
(a) Evidence of disease progression based on RECIST 1.1 Criteria in dose escalation phase.
(b) Noncompliance.
(c) Subject voluntarily withdrew his/her consent.
(d) Subject develops a DLT.
(e) Extraordinary medical circumstances: Subject who, in the opinion of the Investigator, should be discontinued for their well-being or if at any time the treatments prescribed by this protocol are detrimental to the subject's health, the subject may be withdrawn from the study. In this event, reasons for withdrawal should be clearly documented.
(f) If subject becomes pregnant or requires breast-feeding during the Treatment period.
(g) Subjects who are off protocol therapy instead of disease progression will need to complete End of Study Visit.
(h) All surviving subjects were followed up for survival status by phone contact or subjects' clinic visit every 12 weeks for up to 24 weeks.

Off Study Criteria
(a) Death.
(b) Lost to follow-up: After Investigators or study staff have attempted to reach the subject at least 2 times over a 4-week period and failed, the subject may then be considered lost to follow-up.
(c) Withdrawal of consent: Subject decides to voluntarily withdraw from the study AND refuses collection of follow-up information.

Statistical Considerations

Target Sample Size

A maximum number of 18 subjects can be enrolled in the dose escalation phase; and a maximum number of 14 subjects can be enrolled in the cohort expansion phase. A maximum of total 32 subjects can be enrolled in this Phase I study. The sample sizes for the study are not driven by statistical considerations. The study is considered as pilot and exploratory in nature to evaluate the potential dose-response relationship to facilitate dose selection for subsequent studies.

Safety and Toxicity

Toxicity graded by the US NCI Common Toxicity Criteria Version 4.0 in conjunction with MedDRA (Medical Dictionary for Regulatory Affairs) was employed to evaluate the safety profile of the study treatments.

Statistical Methods

Analytical Sets
(a) The Safety Population is the group of subjects who received at least one dose of investigational product and had at least one post-dose safety assessment.
(b) The DLT Evaluation Population included subjects in the Safety Population and excluded those who met the following criteria: the reason of not completing 5 injections was other than development of related AE or a DLT (e.g. noncompliance or withdrawal of consent) in the dose escalation phase.
(c) The Immune Response Population is the group of subjects who received at least one dose of investigational product and had anti-Globo H IgG or IgM production.
(d) The Tumor Response Population will include all enrolled subjects who received at least one dose of investigational product.

Premature Termination and Missing Values

All available data was displayed and utilized in data analysis. Subjects prematurely terminating the study treatment was summarized. Listings of subjects with premature termination was provided with the dates and reasons for termination. Missing data will not be replaced by any estimated or imputed values.

Baseline

The baselines for clinical findings, laboratory evaluations, vital signs, physical examination, ECG, and performance status, as detailed in Table 1 and 2—Treatment and Post Treatment Periods, was evaluated at entry.

Safety Analysis

Adverse Events

Adverse events were regarded as Treatment Emergent (TEAE) if they started on or after the date and time of administration of the first dose of study drug, or if they were present prior to the administration of the first dose of study drug and increased in severity during the study.

Adverse Events (AEs) were coded using the standard Medical Dictionary for Regulatory Activities (MedDRA) dictionary and grouped by system organ class and preferred term and events. TEAEs were summarized by frequency and proportion of total subjects, by system organ class and preferred terms. Separate summaries were given for: all events, events by CTC grade, and events by relationship to study drug. All AEs were provided in data listings.

Subjects who died during the study were summarized and listed. Subjects with Serious Adverse Events (SAEs) were summarized and listed. AEs leading to discontinuation or leading to modification of drug dose were summarized.

Clinical Laboratory Parameters

Each laboratory analyses was summarized using descriptive statistics mean (standard deviation), median (range, min, max). Change from baseline will also be summarized. The incidence of markedly abnormal lab values were provided.

Other Safety Parameters

Other safety parameters such as vital signs, ECGs, concomitant medications and study drug exposure was summarized via summary tables and descriptive statistics.

Immune Response Analysis

The results of anti-Globo H IgG and IgM titer determined by ELISA at each assessment time point per dose cohort was summarized using descriptive statistics. Change from baseline (Week 1) will also be summarized. The maximal response, time to maximal response and the area under the response curve for each IgG type may be determined to aid the evaluation of potential dose-response relationship.

The immune response may also be analyzed and summarized based on the solid tumor cancer type as appropriate, or based on the IHC results of Globo H, SSEA-3, SSEA-4 and PD-L1 antigen at baseline (Week 1 or screening).

Exploratory Analysis of Other Biomarkers

The results of exploratory analysis of other biomarkers (Anti-SSEA-3, anti-SSEA-4 antibodies, CTC, ADCC, CDC) at each assessment time point per dose cohort was summarized using descriptive statistics. Change from baseline (Week 1) will also be summarized. Cellular immune responses (B cells and T cells) were analyzed for 4 continuous visits after either serum anti-Globo H, anti-SSEA-3, or anti-SSEA-4 IgG reaches 20 μg/ml after Week 8. B/T cell immunogenomic analysis were conducted at Screening, Weeks 6, 12, 20, 64, and every 8 weeks until 8 weeks after 1st disease progression. Ex vivo immunogenicity Analysis was conducted at screening visit. Anti-Globo H, anti-SSEA-3 and anti-SSEA-4 antibody was evaluated by Quantitative ELISA and glycan array. Cobas EGFR Mutation Tests were conducted at Weeks 1, 12, 40 and post treatment period 1 for all subjects with known EGFR mutations in selected Taiwan sites.

Adverse Events and Serious Adverse Events

Timely, accurate, and complete reporting and analysis of safety information from clinical trials are crucial for the protection of subjects, Investigators, and the Sponsor, and is mandated by regulatory agencies worldwide. The sponsor has established standard operating procedures (SOPs) in conformity with regulatory requirements worldwide to ensure appropriate reporting of safety information; all clinical trials sponsored by OBI Pharma, Inc. or its affiliates were conducted in accordance with those procedures.

The investigator and/or delegated site staff is responsible for detecting, documenting and reporting events that meet the definition of an Adverse Event (AE) or Serious Adverse Event (SAE). During the study when there is a cohort safety evaluation, the investigator or site staff were responsible for detecting, documenting and reporting all AEs and SAEs. AEs and SAEs were collected from the start of dosing of OBI-833/OBI-821 and until the End of Study.

Adverse Events

Based on ICH guidelines, an adverse event (AE) is defined as any untoward medical occurrence in a clinical study subject administered a medicinal product. An AE does not necessarily have a causal relationship with the treatment. An AE can therefore be any unfavorable and unintended sign (including an abnormal finding), symptom, or disease temporally associated with the use of a medicinal product, whether or not related to the investigational drug. This includes any occurrence that is new in onset or aggravated in severity or frequency from the baseline condition, or abnormal results of diagnostic procedures, including laboratory test abnormalities.

Treatment-Related AEs

Adverse events (AEs) and toxicities were assessed throughout the study and graded according to US NCI Common Toxicity Criteria, Version 4.0, developed by the Cancer Therapy Evaluation Program at the National Cancer Institute. The criteria for unacceptable toxicities should include any Grade 4 toxicity, with the exception of local skin reactions, fever, chilling, sweats, urticaria, and/or pruritis since these are common side effects of antibody/adjuvant administration, are reversible, and controlled by supportive management. Theoretically, immune complex disease as manifested by skin, joint, renal, or other manifestations could occur, but these should be rare in the absence of prior exposure to mouse protein. These were an indication to stop therapy in the affected subjects, but accrual of new subjects may continue.

In general, Grade 1 (Mild) and Grade 2 (Moderate) adverse events are considered acceptable. Grade 3 AEs are Severe but reversible or medically manageable conditions involving major organ and organ function. Grade 4 AEs are Life-threatening consequences; urgent intervention indicated.

Any adverse event must be recorded in the subject medical records and on the eCRFs. The onset and end dates, severity, duration, effect on investigational drug administration (e.g., discontinuation), relationship to investigational drug, and administration of any other drug(s) for treatment of AEs were recorded for each adverse event.

Subjects were questioned and/or examined by the Investigator or his/her designee for evidence of adverse events. The questioning of subjects with regard to the possible occurrence of adverse events were generalized as, “How have you been feeling since your last visit?” The presence or absence of specific adverse events should not be solicited from subjects.

Anticipated Adverse Events

Previous reports and clinical studies on Globo H did not present with any drug-related serious adverse effects. Adverse effects were generally mild to moderate with the most common adverse effects been mild flu-like symptoms and transient local skin reactions at the subcutaneous injection site. However, possibilities of an allergic or autoimmune reaction may occur, as with all immunotherapy and vaccines. When serious toxicities due to immunotherapy treatment do occur, please follow the guidelines for treatment discontinuation procedure.

Assessment of Relationship to Treatment

The Investigator must assess the relationship of any adverse event to the use of investigational drug, based on available information, using the following guidelines:
(a) Not related:

With no temporal relationship with administration of the investigational drug. May have negative dechallenge and rechallenge information. Typically explained by extraneous factors (e.g., concomitant disease, environmental factors, or other medications or chemicals).
(b) Unlikely related:

With a temporal relationship to administration of the investigational drug that makes a causal relationship improbable, and in which other medications, chemicals, or underlying disease provide plausible explanations.
(c) Possibly related:

With a reasonable time sequence to administration of the investigational drug, but which could also be explained by concurrent disease or other medications or chemicals. Information on treatment withdrawal may be lacking or unclear.
(d) Probably related:

With a reasonable time sequence to administration of the investigational drug, unlikely to be attributed to concurrent disease or other medications or chemicals, and which follows a clinically reasonable response on withdrawal (dechallenge).
(e) Definitely related:

Occurs in a plausible time relationship to administration of the investigational drug, and which concurrent disease or other medications or chemicals cannot explain. The response to withdrawal of the treatment should be clinically plausible.

Serious Adverse Events

Based on ICH guidelines, a SAE is defined as any untoward medical occurrence that at any dose:
(a) Results in death
(b) Is life-threatening (The subject was at risk of death at the time of the event. It does not refer to an event that hypothetically might have caused death if it were more severe.
(c) Results in persistent or significant disability/incapacity
(d) Requires inpatient hospitalization or results in prolongation of ER visit (≥24 hrs).
(e) Results in congenital abnormally/birth defect
(f) Are medically significant* *Any important medical events that may not be immediately life-threatening or result in death or hospitalization (≥24 hrs) but may jeopardize the subject or may require intervention to prevent one of the outcomes listed in the definition above.

All SAEs that have not resolved by the end of the study, or that have not resolved upon discontinuation of the subject's participation in the study, must be followed until any of the following occurs:
(a) The event resolves
(b) The event stabilizes
(c) The event returns to baseline, if a baseline value/status is available
(d) The event can be attributed to another agent(s) other than the study treatment(s) or to factors unrelated to study conduct
(e) It becomes unlikely that any additional information can be obtained (subject or health care practitioner refusal to provide additional information, lost to follow-up after demonstration of due diligence with follow-up efforts)

Any event requiring hospitalization (or results in prolongation of hospitalization) that occurs during the course of a subject's participation in a clinical study must be reported as a SAE except hospitalizations for the following:
(a) Social reasons in the absence of an AE
(b) Surgery or procedure planned before entry into the study (must be documented in the CRF)
(c) Elective hospitalization for treatment of disease (e.g., subjects be treated with the first dose of the investigational drug)

Disease progression should not be recorded as an AE or SAE term; instead, signs and symptoms of clinical sequelae resulting from disease progression/lack of efficacy were reported if they fulfill the SAE definition.

Reporting of Serious Adverse Events

OBI Pharma, Inc. or its designee must be notified of the occurrence of all serious adverse events whether or not deemed drug related or expected within 24 hours of awareness of the event by the Investigator. This reporting timeframe also applies to serious adverse events follow up report for new information update.

Serious adverse events (SAEs) require immediate notification to OBI Pharma, Inc. or its designee starting from the date of first dosing and until the last follow up visit in the post-treatment period. For subjects who withdraw treatment during the treatment period, the serious adverse events should be reported to OBI Pharma, Inc. through 28 days after the last administration of study product.

However, if any serious adverse events occurred after the reporting period defined above, it is required to be reported within 24 hours of awareness if a causal relationship is suspected.

As for health authority, all serious adverse events report were submitted and followed per local regulations.

The sponsor (OBI Pharma Inc.) will also be responsible for compliance with applicable portions of the USA Public Health Service Act, the Federal Food, Drug, and Cosmetic Act, and the Code of Federal Regulations (CFR). These responsibilities include (1) reporting any unexpected fatal or life-threatening adverse experience associated with use of the product by fax no later than 7 calendar days after initial receipt of the information [21 CFR 312.32(c)(2)]; (2) reporting any adverse experience associated with use of the product that is both serious and unexpected in writing no later than 15 calendar days after initial receipt of the information [21 CFR 312.32(c)(1)]; and submitting annual progress reports (21 CFR 312.33)

Pregnancy

If it is subsequently discovered that a subject is pregnant during the study period, study treatment was permanently discontinued in an appropriate manner.

The Investigator must notify OBI Pharma, Inc. or its designee of this event within 24 hours of awareness.

The pregnancy events require immediate notification to OBI Pharma, Inc. or its designee starting from the date of first dosing until the last follow up visit in the post-treatment period. For the subject who withdraws the study during the treatment period, the pregnancy events should be reported to OBI Pharma, Inc. until 28 days after the last administration of study product.

In addition, the Investigator must report to OBI Pharma, Inc. or its designee follow-up information regarding the course of the pregnancy, including perinatal and neonatal outcome.

Data and Safety Monitoring Board

A Data and Safety Monitoring Board was established to assist the Sponsor in monitoring subject safety, risk/benefit, and decision for dose escalation, dose modification and termination of the study.

Study Administration and Monitoring

Institutional Review Board Approval

This proposed study must have the approval of a properly constituted Institutional Review Board (IRB). Investigator will obtain written and dated approval from the IRB for the protocol, protocol amendment, informed consent forms, recruitment materials and any other written information to be provided to the subjects.

Informed Consent Forms

Each subject (or a legally authorized representative) must give written consent (and sign other locally required documents) according to local requirements after the nature of the study has been fully explained. The consent form must be signed prior to performance of any study-related activity. The consent form that is used must be approved both by the sponsor and by the reviewing IEC/IRB. The informed consent should be in accordance with the current revision of the Declaration of Helsinki, current International Conference on Harmonization (ICH) and Good Clinical Practice (GCP) guidelines, and OBI Pharma, Inc. or its designee's policy.

The Investigator or person obtaining consent according to the institutional policies and procedures must explain to potential subjects or their legal representatives the aims, methods, reasonably anticipated benefits and potential hazards of the trial and any discomfort the subjects may experience. Subjects were informed that they are free not to participate in the trial and that they may withdraw consent to participate at any time. They were told which alternative treatments are available if they refuse to take part and that such refusal will not prejudice future treatment. Finally, they were told that their records may be examined by competent authorities and authorized persons, but that personal information would be treated as strictly confidential and will not be publicly available. Subjects must be given the opportunity to ask questions. After this explanation and before entry into the trial, consent should be appropriately recorded by means of the subject's or his/her legal representative's dated signature. If a subject and his/her legal representative are unable to read, the consenting process was conducted according to the institution policies and procedures.

The subject should receive a signed and dated copy of the informed consent form. A copy of the signed informed consent form (including amended consents) must be given to the subject prior to the study participation. The Investigator must keep each subject's signed consent form(s) on file and readily available for review by the monitor and for inspection by the regulatory agency at any time.

Study Conduct and Monitoring

All aspect of the study was conducted under ICH and Good Clinical Practice guidelines.

It were monitored by qualified individuals designated by the sponsor. Monitoring was conducted according to Good Clinical Practice and standard operating procedures for compliance with applicable government regulations. The Investigator will agree to the monitor's access to the clinical supplies, dispensing, and storage area, and to the clinical files of the study subjects, and if requested, agrees to assist the monitor.

Case Report Forms

Data from this study was entered in electronic Case Report Forms (eCRFs) using a validated Electronic Data Capture (EDC) system was employed by the Sponsor via a designated Contract Research Organization (CRO). Site personnel will receive detailed training on completion of the eCRFs. All data entered was reviewed electronically at a central location and any discrepancies or clarifications was corrected during routine on-site clinical monitoring.

Concomitant medications entered into the database was coded using the WHO Drug Reference List. Adverse events were coded using the Medical Dictionary for Regulatory Activities (MedDRA) terminology. It is the Investigator's responsibility to ensure completion of all applicable eCRFs and to review and approve all eCRFs according to instructions by the Sponsor designated CRO.

Quality Control and Quality Assurance

To ensure accurate, complete, and reliable data, OBI Pharma, Inc. or its representatives will conduct periodic monitoring visits to ensure that the protocol and GCPs are being followed. The monitors will review source documents and ensure the data recorded in eCRF are accurate. The site may be subjected to review by the IRB and/or to quality assurance audits performed by OBI Pharma, Inc. or its representatives.

Drug Accountability

It is the responsibility of the clinical Investigator to ensure that all study drug received at the site was inventoried and accounted for throughout the study and recorded in the drug accountability forms maintained in the Trial Center File. The drug accountability was verified by the monitor during on site monitoring visits. Study drug was stored in a limited access area according to temperature specifications.

The Investigator will confirm that all original containers are retained and stored according to the institutional policy, until these containers are inventoried by the sponsor. Original containers will not be retained and stored if not allowable according to institutional policy. Unless otherwise instructed by the sponsor, the Investigator agrees at the end of the study to return all retained containers of study drug to the sponsor as instructed by the site manager. A pharmacist of the medical center or the authorized personnel designated by the Investigator will fill out the drug accountability records. All entries must be legible and complete.

OBI Pharma, Inc. or its designee will ensure proper disposition of original containers empty or full with returned or unused study drug. Appropriate documentation will be maintained. If OBI Pharma, Inc. authorizes destruction at the trial site, the Investigator must ensure that the materials are destroyed in compliance with applicable regulation policy, according to the institution's destruction policy and any instructions provided by OBI Pharma, Inc.

Study Completion/Discontinuation/Termination

The following situations are regarded as study completion:
(a) Successful completion of the trial at the center;
(b) The required number of subjects for the trial has been recruited.

Study termination can occur at any time either by the Sponsor or by the Investigator, provided there is reasonable cause and sufficient notice is given in advance of the intended termination. Reasons for such action taken by the Sponsor may include, but are not limited to:
(a) Failure of the Investigator to comply with the protocol, the Sponsor's procedures or GCP guidelines;
(b) Ethical concerns;
(c) Safety and/or toxicity concerns;
(d) Sufficient data suggesting lack of efficacy;
(e) Inadequate recruitment of subjects by the Investigator

### Example 1: OBI-833 and OBI-821 Mixing Instruction for Subcutaneous Administration

Storage Condition: Both OBI-833 and OBI-821 vials are to be stored at 2-8° C.

Study Cohorts
Cohort 1: OBI-833 (10 μg Globo H/100 μg OBI-821)
Cohort 2: OBI-833 (30 μg Globo H/100 μg OBI-821)
Cohort 3: OBI-833 (100 μg Globo H/100 μg OBI-821)

Investigational Drugs:

Orange Vial (OBI-821)

Contents: 125 μg OBI-821 in sodium phosphate buffered saline, and Trehalose. At time of treatment, with a syringe, add 0.5 mL or 0.9 mL of water for injection to Orange Vial (OBI-821) to obtain OBI-821 solution: Fully dissolve the contents of the vial (Orange Vial)) by gently inverting the vial 4-5 times. Do not shake the vial vigorously. According to drug mixing procedures for three cohorts, OBI-821 is mixed with OBI-833 immediately for injection after re-constituted with WFI.

TABLE 2
OBI-833/OBI-821 Orange Vial Preparation

| | Water for |
|---|---|
| | Injection (mL) |
| | added to |
| Dosing Cohort | Orange Vial |
| Cohort 1: OBI-833 (10 μg Globo H/100 μg OBI-821) | 0.9 |
| Cohort 2: OBI-833 (30 μg Globo H/100 μg OBI-821) | 0.5 |
| Cohort 3: OBI-833 (100 μg Globo H/100 μg OBI-821) | 0.5 |

Green Vial (OBI-833)

Contents: 150 μg Globo H linked with CRM197 with potassium phosphate buffered saline, Sucrose, and polysorbate 80. At time of treatment, with a syringe, add, according to the respective cohort, 1.2 mL or 2.0 mL of water for injection to Green Vial to obtain OBI-833 solution. Fully dissolve the contents of the vial (Green Vial) by gently inverting the vial 4-5 times. Do not shake the vial vigorously. According to drug mixing procedures for three cohorts, OBI-833 is mixed with OBI-821 immediately for injection after re-constituted with WFI.

TABLE 3
OBI-833/OBI-821 Green Vial preparation

| | Water for |
|---|---|
| | Injection (mL) |
| | added to |
| Dosing Cohort | Green Vial |
| Cohort 1: OBI-833 (10 μg Globo H/100 μg OBI-821) | 1.2 |
| Cohort 2: OBI-833 (30 μg Globo H/100 μg OBI-821) | 2.0 |
| Cohort 3: OBI-833 (100 μg Globo H/100 μg OBI-821) | 1.2 |

At the time of treatment, withdraw the contents of Green vial (OBI-833) and the appropriate amount of water for injection according to the table below and transfer into Orange Vial (OBI-821). Mix the contents of the Orange Vial (OBI-821) by gently inverting the vial 4-5 times. Do not shake the vial vigorously. At this point, Orange Vial (OBI-821) contains the Treatment (OBI-833 plus OBI-821) is ready for injection into the study subject. Withdraw the appropriate volume from Orange Vial (OBI-821) containing the Treatment for injection.

TABLE 4
Study drμg for Orange Vial and Green Vial mixing.

| | Volume of | Total Volume |
|---|---|---|
| | Green Vial (mL) | Administered |
| | to be added to | (mL) from |
| | Orange Vial | Orange Vial |
| Dosing Cohort | (OBI-821) | (OBI-821) |
| Cohort 1: OBI-833 | 0.1 | 0.8 |
| (10 μg Globo H/100 μg OBI-821) | | |
| Cohort 2: OBI-833 | 0.5 | 0.8 |
| (30 μg Globo H/100 μg OBI-821) | | |
| Cohort 3: OBI-833 | 1.0 | 1.2 |
| (100 μg Globo H/100 μg OBI-821) | | |

*WFI = Water for Injection

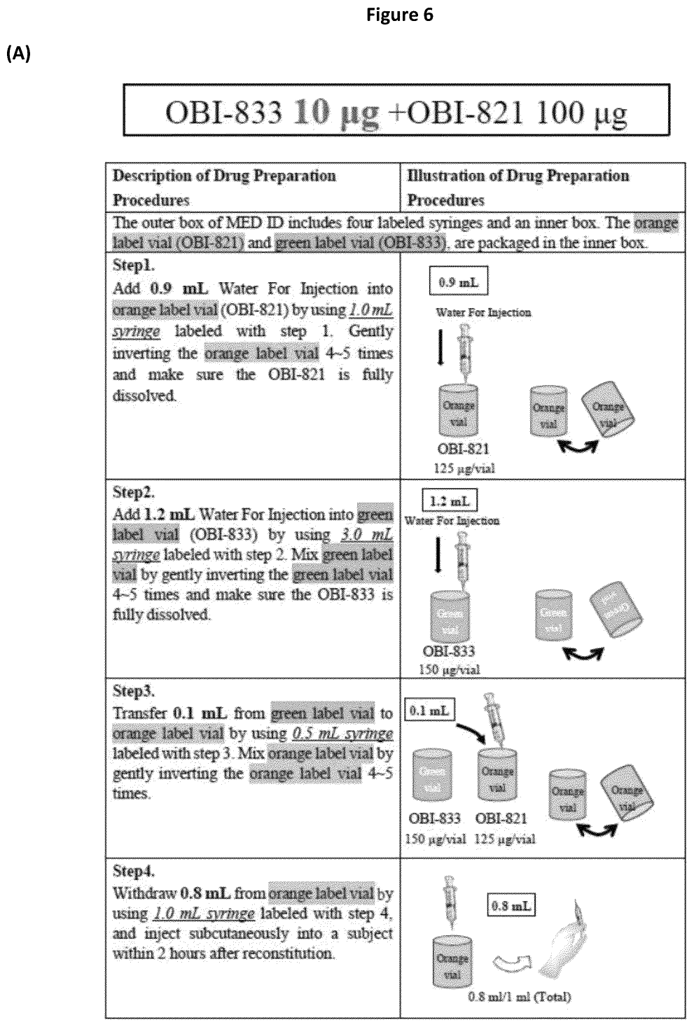

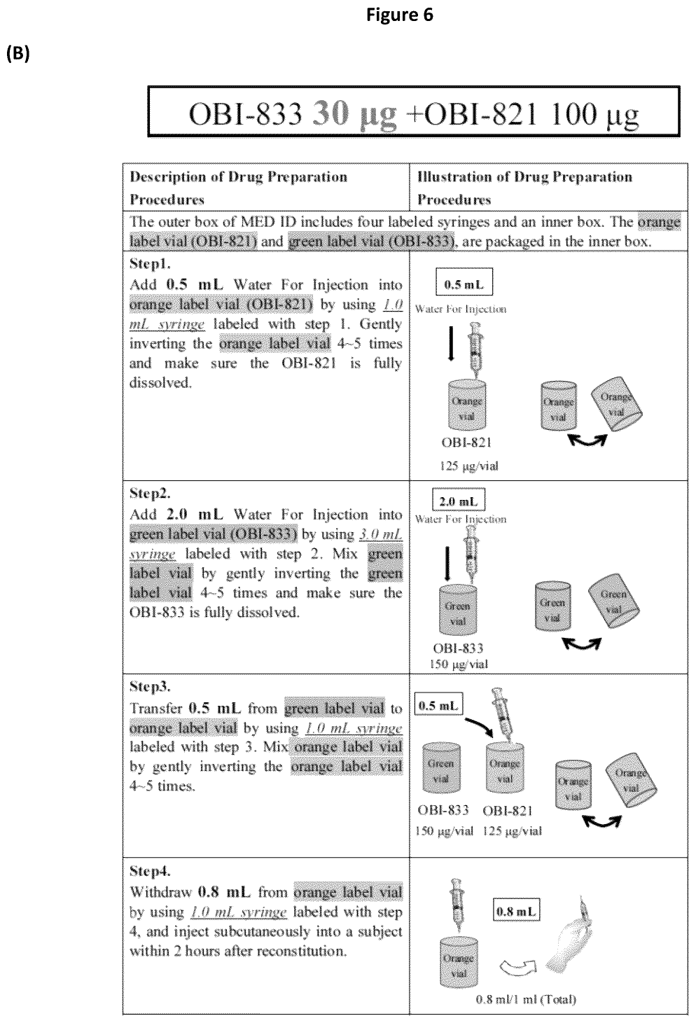

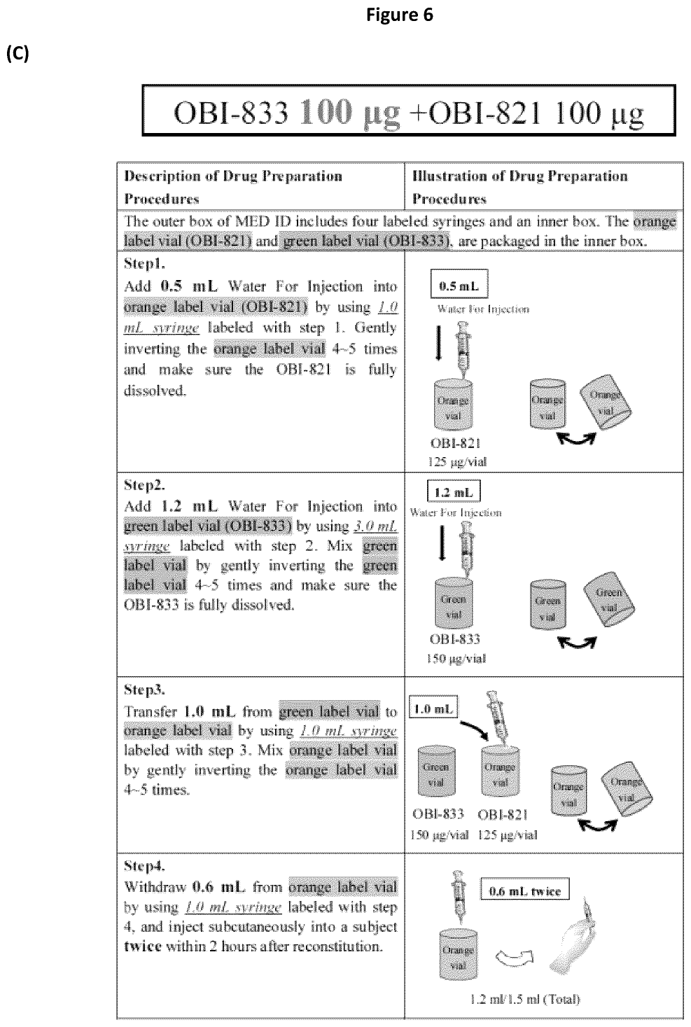

FIG. 6 illustrates the study drug mixing procedures in Cohort 1, Cohort 2 and Cohort 3. It is highly recommended that the administration of the combined product should occur within 2 hours from reconstitution to minimize potential microbial growth. If administration is not possible within 2 hours from reconstitution, the combined product should be destroyed according to the institutional pharmacy Standard Operating Procedure and documented in the drug accountability records.

Table 5 indicated the prohibited concomitant medication during Dose Escalation Phase and Cohort Expansion Phase.

TABLE 5
Prohibited concomitant medication
during OBI-833-001 clinical trial
[Dose Escalation Phase]
Chemotherapeutic Agents:

| | |
|---|---|
| Antimetabolites | Miscellaneous cytotoxic agents |
| Vinca alkaloid | Alkylating agents |
| Epipodophyllotoxins | Antitumor antibiotics |
| Taxanes | Nitrosoureas |
| Camptothecins | |

Biologic Agents:
Monoclonal antibodies (trastuzumab, cetuximab, etc.)
Interferons
Interleukins
Immunotherapy
cyclosporin, rapamycin, tacrolimus, rituximab, PD-1/PD-L1
antagonists
IV/oral steroid except single prophylactic use in CT/MRI scan,
or other one-time use in approved indications. Inhaled and
topical use of steroids are allowed.
Hormone therapy
Estrogen receptor antagonists, selective estrogen receptor
inhibitors and aromatase inhibitors
Target therapy
Tyrosine kinase inhibitors and monoclonal antibodies
Other non-cancer vaccine therapy
Washout period for concomitant vaccines is two weeks prior to IP
treatment.
Alternative and complementary medicine may affect immune system
[Cohort Expansion Phase]
Chemotherapeutic Agents:

| | |
|---|---|
| Antimetabolites | Miscellaneous cytotoxic agents |
| Vinca alkaloid | Alkylating agents |
| Epipodophyllotoxins | Antitumor antibiotics |
| Taxanes | Nitrosoureas |
| Camptothecins | |

Biologic Agents:
Monoclonal antibodies (except necitumumab and bevacizumab)
Interferons
Interleukins
Immunotherapy
cyclosporin, rapamycin, tacrolimus, rituximab (except PD-1/PD-L1
antagonists)
IV/oral steroid except single prophylactic use in CT/MRI scan, or other
one-time use in approved indications. Inhaled and topical use of steroids
are allowed.
Other non-cancer vaccine therapy
Washout period for concomitant vaccines is two weeks prior to IP
treatment.
Alternative and complementary medicine may affect immune system
Subjects who continuous treatment after disease progression are
allowed to concomitant use of all approved anti-cancer therapies

Eastern Cooperative Oncology Group (ECOG) Performance.

These scales and criteria are used by doctors and researchers to assess how a subject's disease is progressing, assess how the disease affects the daily living abilities of the subject, and determine appropriate treatment and prognosis. They are included here for health care professionals to access. They are included in table 6 for health care professionals to access.

TABLE 6
ECOG performance status of OBI-833-001 clinical trial
ECOG PERFORMANCE STATUS*

| Grade | ECOG |
|---|---|
| 0 | Fully active, able to carry on all pre-disease performance without |
| | restriction |
| 1 | Restricted in physically strenuous activity but ambulatory and |
| | able to carry out work of a light or sedentary nature (e.g., |
| | light house work, office work) |
| 2 | Ambulatory and capable of all self-care but unable to carry out |
| | any work activities. Up and about more than 50% of waking |
| | hours |
| 3 | Capable of only limited self-care, confined to bed or chair more |
| | than 50% of waking hours |
| 4 | Completely disabled. Cannot carry on any self-care. Totally |
| | confined to bed or chair |
| 5 | Dead |

*As published in Am. J. Clin. Oncol.:
Oken, M. M., Creech, R. H., Tormey, D. C., Horton, J., Davis, T. E., McFadden, E. T., Carbone, P. P.: Toxicity And Response Criteria Of The Eastern Cooperative Oncology Group. Am J Clin Oncol 5: 649-655, 1982.
The ECOG Performance Status is in the public domain therefore available for public use. To duplicate the scale, please cite the reference above and credit the Eastern Cooperative Oncology Group, Robert Comis M.D., Group Chair.

### Example 2: Results of the Dose Escalation Phase of the OBI-833-001 Phase 1 Study

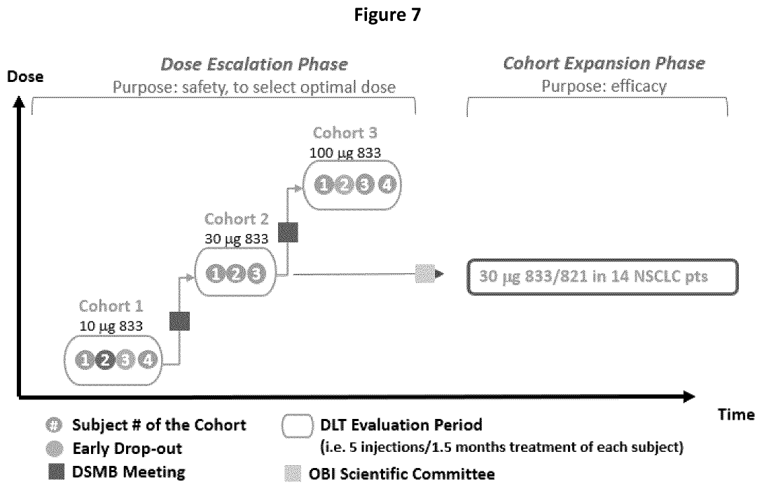

FIG. 7 illustrates the design schema (Dose Escalation Phase and Cohort Expansion Phase) of OBI-833-001 clinical study. The study purpose of Dose Escalation Phase is safety and selecting optimal dose. The study purpose of Cohort Expansion Phase is efficacy.

Recurrent/metastatic incurable gastric, lung, colorectal, or breast cancer patients without SOC in US & TW protocol. For patients with metastatic lung and breast cancer subjects who achieved CR/PR/SD after at least 1 regimen of anticancer therapy are permitted maintenance hormone and/or target therapies in Taiwan protocol. The details of the patients enrolled in Dose Escalation Phase were described in Table 7. Of the 16 subjects who were screened, 11 subjects were enrolled in the dose escalation phase: 4 subjects in Cohort 1 (10 μg OBI 833/100 μg OBI-821), 3 subjects in Cohort 2 (30 μg OBI 833/100 μg OBI-821), and 4 subjects in Cohort 3 (100 μg OBI 833/100 μg OBI-821). Four out of the 5 screen failures were because of non-fulfilment of the inclusion/exclusion criteria; 1 screen failure was due to death during the screening process. One subject had stable disease and 2 subjects had progressive disease as the best tumor response in Cohort 1. All 3 subjects in Cohort 2 had progressive disease as the best tumor response. One subject had stable disease and 1 subject had progressive disease as the best tumor response in Cohort 3.

TABLE 7
Subject disposition in the Dose Escalation Phase

| Number of | Cohort 1 | Cohort 2 | Cohort 3 |
|---|---|---|---|
| Patients | (10 μg 833) | (30 μg 833) | (100 μg 833) |
| Enrolled | 4 | 3 | 4 |
| Race | White: 3 | Black or African | White: 3 |
| | Asian: 1 | American: 2 | Asian: 1 |
| | | White: 1 | |
| Gender | Female: 3 | Female:3 | Female: 3 |
| | Male: 1 | | Male: 1 |
| Cancer Types | 1*NSCLC; | 3* Breast | 4* CRC |
| | 3* CRC | | |
| SAE | 1^{#} | 0 | 2^{#} |
| Stable Disease | 1 | 0 | 1 |
| (SD) | | | |
| Progressive | 2 | 3 | 1 |
| Disease (PD) | | | |
| Missing | 1* | 0 | 2* |
| List of Study | 4 | 3 | 4 |
| Discontinuation | | | |

^{#}Subject 015-002 had an SAE due to acute respiratory failure; Subject 015-005 had an SAE death due to disease progression of the underlying cancer, and Subject 015-006 had an SAE due to bilateral malignant pleural effusion. All SAEs were not treatment-related.

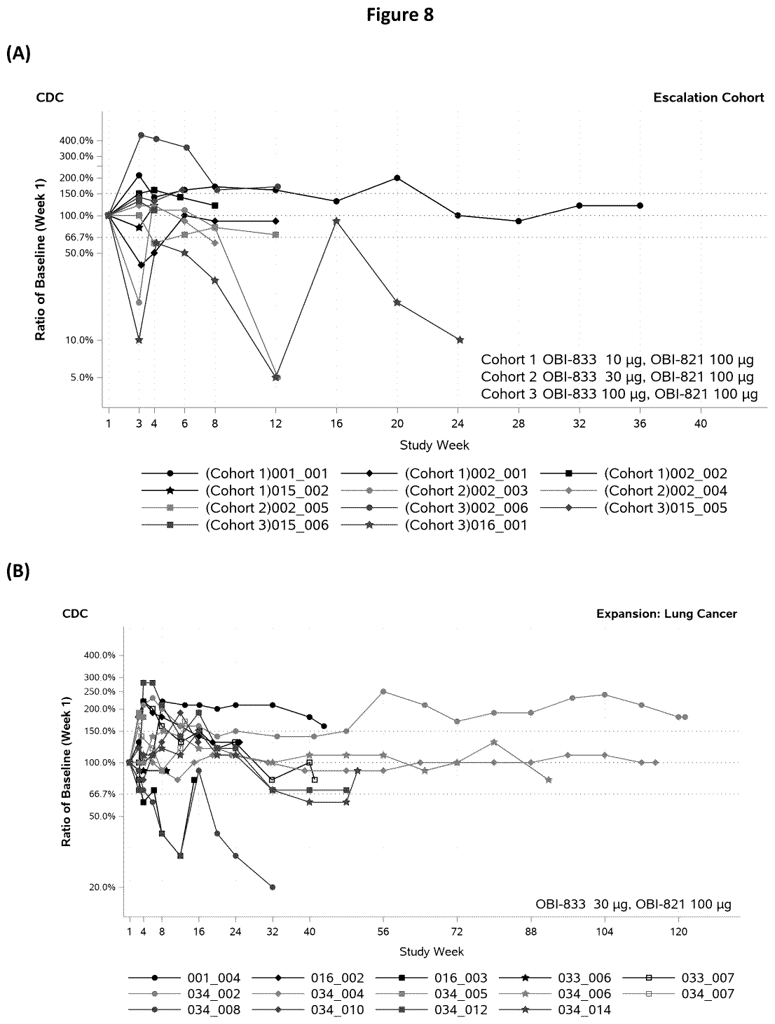

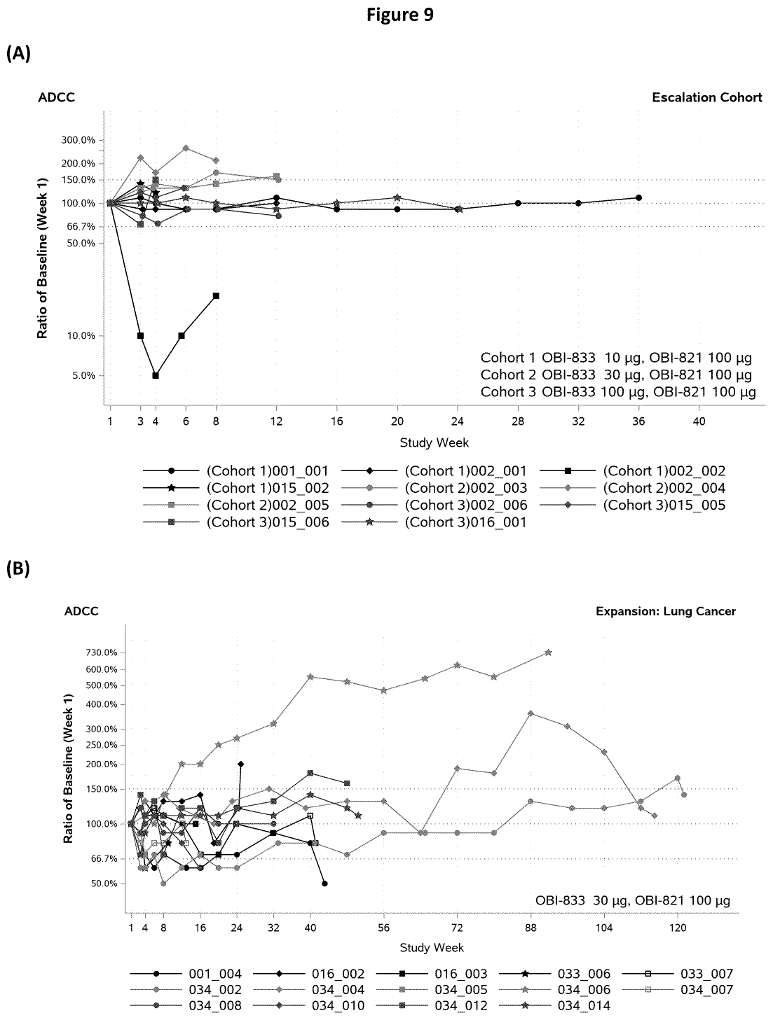

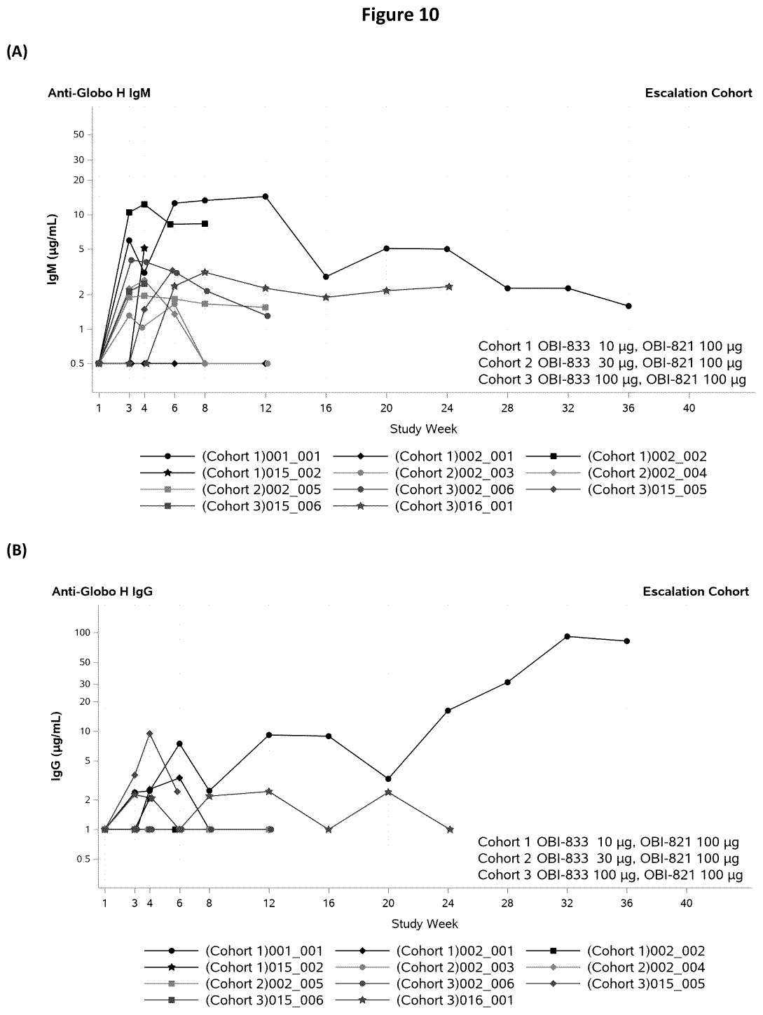

There were in total 11 patients enrolled, including 4 patients in 10-μg, 3 patients in 30-μg and 4 patients in 100-μg dose groups. FIG. 8 illustrates CDC results and FIG. 9 illustrates ADCC results. They both indicated OBI-833 could induce CDC and ADCC effect. FIG. 10 illustrates the immune responses in the dose escalation phase. At baseline, the anti-Globo H IgM levels in all subjects in the dose escalation phase were below the detection limit of 1.0 μg/mL. All but 1 subject in Cohort 1 showed detectable anti Globo H IgM levels at least once at the study visits, with the highest geometric mean anti-Globo H IgM levels in Cohort 1 from the Week 3 visit to the Week 8 visit (range: 1.984 μg/mL to 3.805 μg/mL) as compared to those in Cohort 2 (range: 0.746 μg/mL to 1.773 μg/mL) and Cohort 3 (range: 1.206 μg/mL to 2.870 μg/mL).

At baseline, the anti-Globo H IgG levels in all subjects in the dose escalation phase were below the detection limit of 2.0 μg/mL. Five subjects (3 subjects in Cohort 1 and 2 subjects in Cohort 3) showed detectable anti-Globo H IgG levels at least once in the study visits. Anti-Globo H IgG was not detected in all 3 subjects in Cohort 2. The geometric mean anti-Globo H IgG levels in Cohort 1 from the Week 3 visit to the Week 8 visit (range: 1.242 μg/mL to 2.918 μg/mL) were comparable with those in Cohort 3 (range: 1.343 μg/mL to 2.103 μg/mL). Notably, only 1 subject in Cohort 1 and 1 subject in Cohort 3 in the dose escalation phase remained in the treatment and/or post-treatment period after Week 12 visit. The anti Globo H IgG level increased to as high as 91.18 μg/mL at the Week 32 visit in the subject in Cohort 1 (Subject 001-001).

### Example 3: Results of the Cohort Expansion Phase of the OBI-833-001 Phase 1 Study

Patients with Globo H-positive, metastatic NSCLC who had achieved SD or PR after at least one regimen of therapy. For patients who were on the targeted or anti-PD-1/PD-L1 therapy, OBI-833 was added to their ongoing therapies. The details of 14 NSCLC patients enrolled in the cohort expansion phase are described in Table 8. Of the 24 subjects who were screened, 14 subjects were enrolled in the cohort expansion phase. All screen failures were due to non-fulfilment of the inclusion/exclusion criteria. No subject in the cohort expansion phase achieved complete response or partial response as the best tumor response. One subject (Subject 034-005) died before any post-baseline RECIST evaluation. Eight subjects had an overall RECIST response of progressive disease at the end of the study. Five subjects had an overall RECIST response of stable disease at the end of the study. One (Subject 034-012) of these 5 subjects had an overall RECIST response of stable disease at the Week 12 and Week 24 visits, which worsened to progressive disease at the Post Treatment 1 visit. Further tumor response was stable disease at the Post Treatment 2 and End of Study (Final Injection) visits. One subject (Subject 034-010) was reported with stable disease at the Week 12 visit (Visit 7; Day 85). However, multiple new bone lesions were revealed in the bone scan report on Day 88. Therefore, the Investigator assessed the subject's final tumor response in the study as progressive disease.

TABLE 8
Subject disposition in the Cohort Expansion Phase

| | Subject No | | | | | | |
|---|---|---|---|---|---|---|---|
| | 001-004 | 034-002 | 034-004 | 034-005 | 034-006 | 034-007 | 034-008 |
| Gender | Female | Female | Female | Male | Male | Female | Male |
| Age | 63 | 51 | 68 | 50 | 64 | 61 | 81 |
| ECOG | 1 | 0 | 0 | 0 | 0 | 0 | 1 |
| ConMed | Erlotinib | Erlotinib | Gefitinib | Afatinib | Afatinib | N/A | Erlotinib |

| | Subject No | | | | | | |
|---|---|---|---|---|---|---|---|
| | 033-006 | 033-007 | 034-010 | 034-012 | 034-014 | 016-002 | 016-003 |
| Gender | Female | Male | Male | Male | Male | Female | Female |
| Age | 58 | 62 | 61 | 46 | 56 | 44 | 70 |
| ECOG | 0 | | 0 | 0 | 0 | 0 | 0 |
| ConMed | N/A | Afatinib + | Afatinib | Afatinib | Afatinib | Ceritinib | Erlotinib |
| | | bevacizumab | | | | | |

Most common treatment-related AEs were grade 1-2 injection site reactions. Only one possibly drug-related acute pancreatitis SAE was reported.

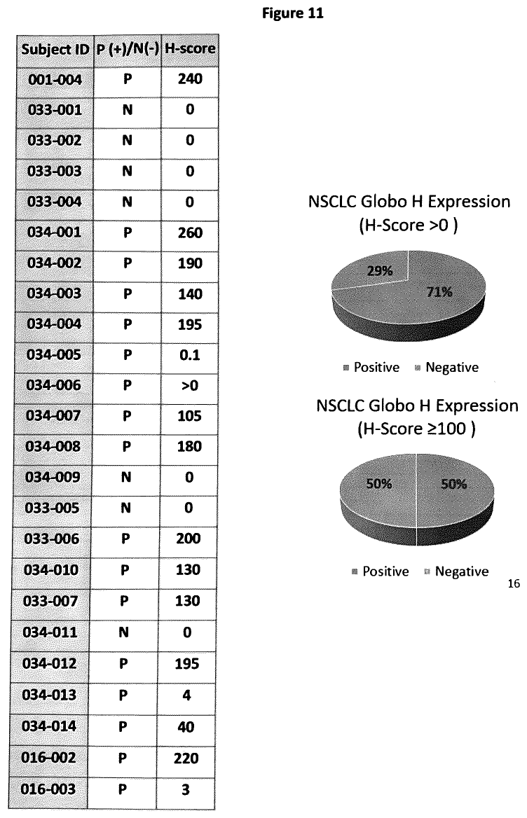

FIG. 11 illustrates the Globo H expression levels (H-score) among 24 NSCLC patients. H-Score calculation: (% cells with 1+intensity)×1+(% cells with 2+intensity)×2+(% cells with 3+intensity)×3. The range of H score is from 0 to 300. FIG. 11 indicated 50% of NSCLC patients were Globo H-positive at the H score cutoff of 100. It meant lung cancer is highly Globo H expressing cancer.

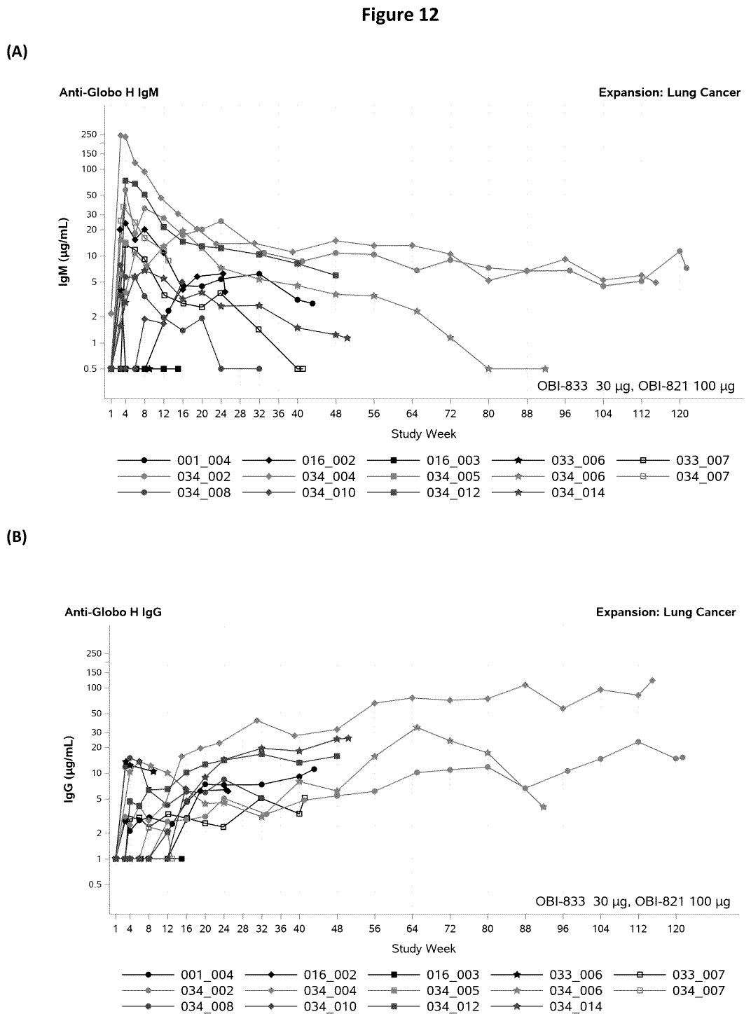

FIG. 12 illustrates the immune response of anti-Globo H IgM/IgG in the cohort expansion phase. At baseline, the anti-Globo H IgM levels in all subjects in the cohort expansion phase were below the detection limit of 1.0 μg/mL, except 1 subject (Subject 034_004) who had an anti-Globo H IgM level of 2.16 μg/mL. All but 1 subject showed detectable anti-Globo H IgM levels at least once in the study visits, with the geometric mean anti-Globo H IgM levels ranging from 3.616 μg/mL to 7.389 μg/mL from the Week 3 visit to the Week 48 visit. Of note, the anti-Globo H IgM levels of Subject 034004 dramatically increased to 242.81 μg/mL at the Week 3 visit and 233.15 μg/mL at the Week 4 visit before gradually decreasing in subsequent weeks. The anti Globo H IgM levels mostly decreased and remained constant after the Week 48 visit. At baseline, the anti-Globo H IgG levels in all subjects in the cohort expansion phase were below the detection limit of 2.0 μg/mL. All but 2 subjects showed detectable anti-Globo H IgG levels at least once in the study visits, with the geometric mean anti-Globo H IgG levels gradually increased from 1.743 μg/mL at the Week 3 visit to 13.351 μg/mL at the Week 48 visit. Notably, less than 4 subjects in the cohort expansion phase remained in the treatment period and/or post-treatment period from Week 56 onwards. The anti-Globo H IgG levels of Subject 034_004 continued to increase from 65.52 μg/mL at Week 56 visit to 120.84 μg/mL at the last visit.

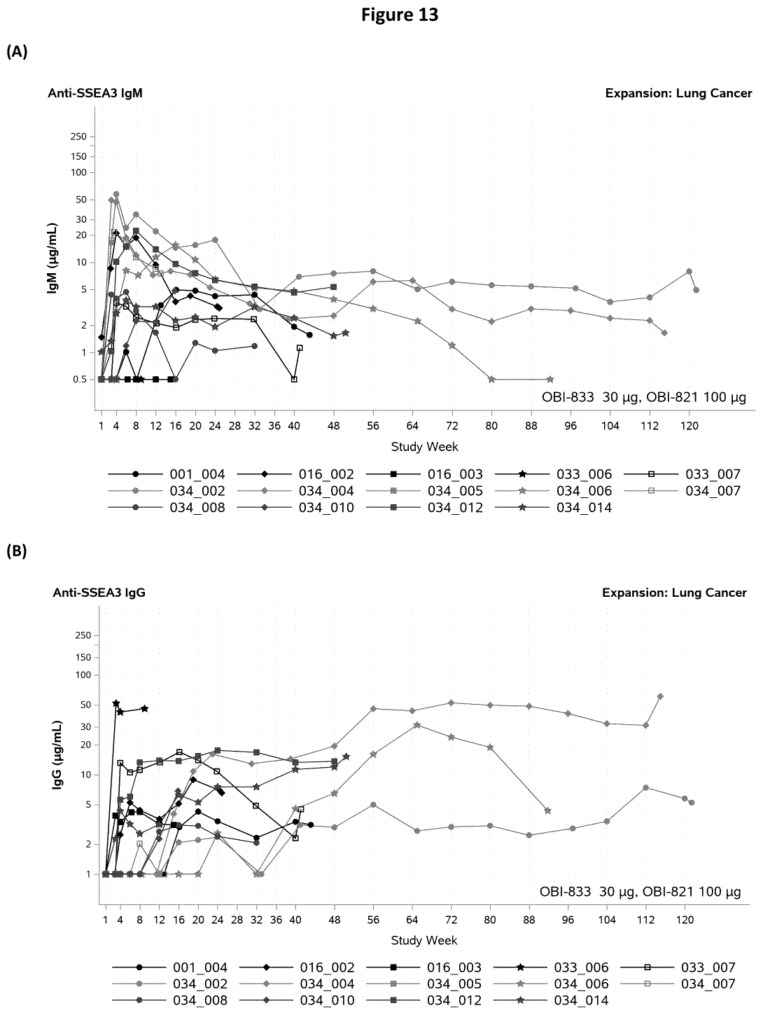

FIG. 13 illustrates the immune response of anti-SSEA3 IgM/IgG in the cohort expansion phase. At baseline, the anti-SSEA 3 IgM and IgG levels in all subjects were below the detection limit, except 2 subjects (Subject 034_014 and 016_002) who had anti-SSEA 3 IgM levels of 1.01 μg/mL and 1.47 μg/mL, respectively. The geometric mean anti-SSEA 3 IgM levels ranged from 2.048 μg/mL to 5.285 μg/mL from Week 3 visit to Week 48 visit; and the geometric mean anti SSEA 3 IgG levels ranged from 1.601 μg/mL to 9.013 μg/mL from Week 3 visit to Week 48 visit.

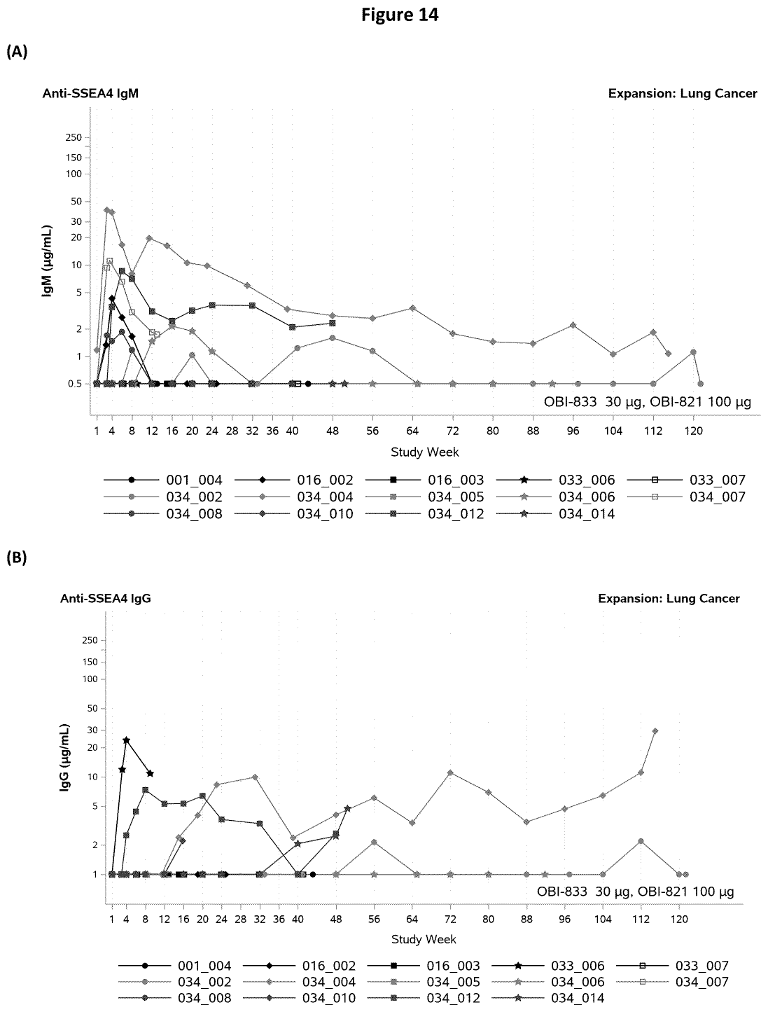

FIG. 14 illustrates the immune response of anti-SSEA4 IgM/IgG in the cohort expansion phase. At baseline, the anti-SSEA 4 IgM and IgG levels in all subjects were below the detection limit, except 1 subject (Subject 034_004) who had an anti-SSEA 4 IgM level of 1.17 μg/mL. The geometric mean anti-SSEA 4 IgM levels ranged from 0.872 μg/mL to 1.348 μg/mL from Week 3 visit to Week 48 visit; and the geometric mean anti-SSEA 4 IgG levels ranged from 1.131 μg/mL to 1.924 μg/mL from Week 3 visit to Week 48 visit.

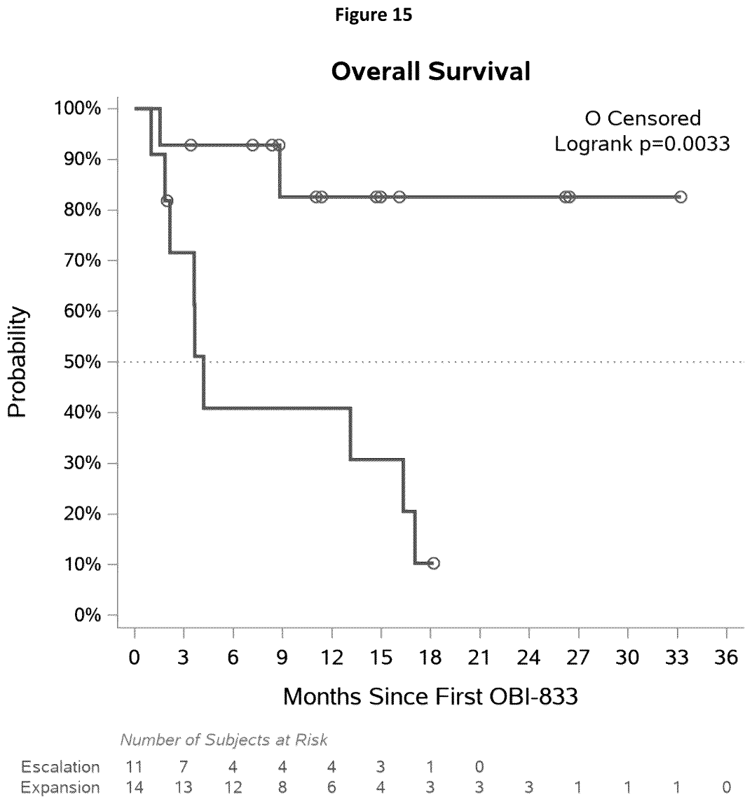

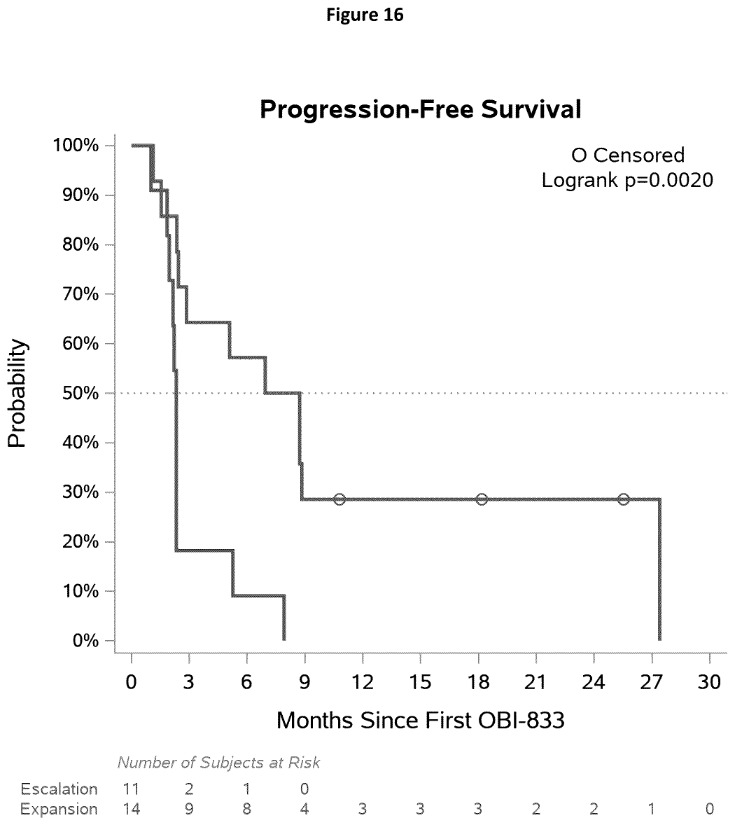

FIG. 15 illustrates the overall survival (OS) and FIG. 16 illustrates the progression-free survival (PFS) in OBI-833-001 dose escalation phase and cohort expansion phase. Three subjects each in Cohort 1 and Cohort 3 and all 3 subjects in Cohort 2 died during the study, with the mean (SD) duration of survival follow-up of 9.1 (8.76) months for Cohort 1, 7.0 (5.32) months for Cohort 2, and 6.5 (7.86) months for Cohort 3. The median time to death as estimated by the Kaplan-Meier method was 16.3 months for Cohort 1, 4.2 months for Cohort 2, and 2.9 months for Cohort 3. The incidence death rate per person-month was the lowest in Cohort 1 (0.082 deaths per person-month [95% CI: 0.017,0.240]), followed by Cohort 3 (0.116 deaths per person-month [95% CI: 0.024,0.339]), and Cohort 2 (0.143 deaths per person-month. Two subjects died during the study, with the mean (SD) duration of survival follow-up of 13.7 (9.17) months. The median time to death could not be estimated by the Kaplan-Meier method due to the small number of deaths. The incidence death rate per person-month was 0.010 deaths per person-month (95% CI: 0.001,0.038) for the cohort expansion phase.

Furthermore, Table 9 indicates that OBI-833 could also induce CDC and ADCC effects for patients who were on the targeted or anti-PD-1/PD-L1 therapy. In most patients, anti-Globo H IgG was evoked and ADCC effects were observed.

TABLE 9
CDC and ADCC values of 14 NSCLC patients who
were on the targeted or anti-PD-1/PD-L1 therapy
Subject ID

033-007
033-006
016-003
016-002
034-014
034-012
034-010
034-008
034-007
034-006
034-005
034-004
034-002
001-004

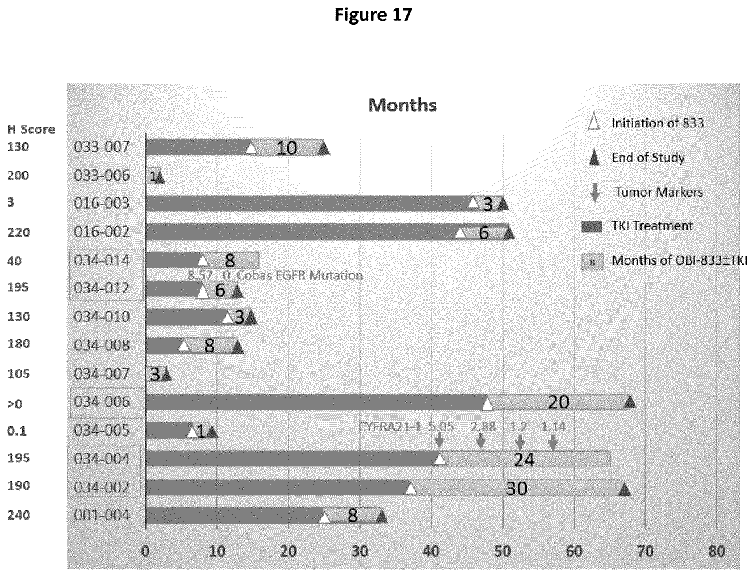

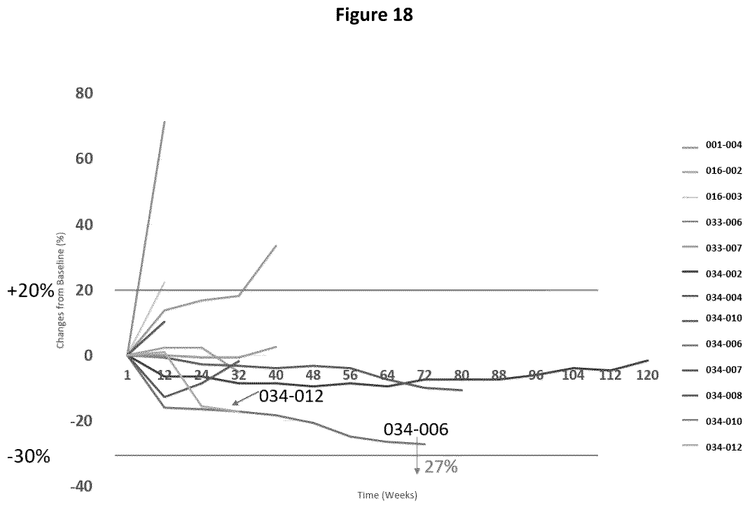

Finally, FIG. 17 illustrates the swimmer plot on EGFR TKIs (tyrosine kinase inhibitors) and OBI-833 treatment duration (from one to thirty months). It indicates that 7 of 11 EGFR TKI-treated patients had SD for over six months. Among them, two patients have been treated for more than two years. On the other hand, FIG. 18 illustrates tumor response over time of NSCLC patients. Among them, one patient's tumor size had reduced by 27% after 16 months of OBI-833 treatment and plasma EGFR mutation load was significantly reduced from 8.57 to 0 in another patient.

In conclusion, no subject achieved the best tumor response as complete response or partial response in this study. However, 1 subject in Cohort 1 and 1 subject in Cohort 3 in the dose escalation phase, and 9 subjects in the cohort expansion phase had (durable) stable disease as the best tumor response. The results of this study could demonstrate the safety, immune response, and preliminary clinical efficacy of OBI-833/OBI-821 in NSCLC patients.

### REFERENCES

1. Allen J R, Harris C R, and Danishefsky S J (2001) Pursuit of optimal carbohydrate-based anticancer vaccines: preparation of a multiantigenic unimolecular glycopeptide containing the Tn, MBr1, and Lewis(y) antigens. J. Am. Chem. Soc. 123: 1890-7
2. Chang, K., Kuo, W., Wang, M. (2008a). The Epidemiology of Breast Cancer in Taiwan. J. Chinese Oncol. 24(2), 85-93
3. Chang W W, Lee C H, Lee P, Lin J, Hsu C W, Hung J T, Lin J J, Yu J C, Shao L, Yu J, Wong C H, and Yu A L. (2008b). Expression of Globo H and SSEA3 in breast cancer stem cells and the involvement of fucosyltransferases 1 and 2 in Globo H synthesis. PNAS (V.105, No. 33): 11667-11672
4. Chapman P B, Morrissey D M, Panageas K S, Hamilton W B, Zhan C, Destro A N, Williams L, Israel R J, Livingston P O (2000a) Induction of antibodies against G_{M2} ganglioside by immunizing melanoma patients using G_{M2}-KLH+QS21 vaccine: a dose-response study. Clin. Cancer Res. 6: 874-879.
5. Chapman P B, Morrisey D, Panageas K S, Williams L, Lewis J J, Israel R J, Hamilton W B, Livingston P O (2000b) Vaccination with a bivalent G(M2) and G(D2) ganglioside conjugate vaccine: a trial comparing doses of G(D2)-keyhole limpet hemocyanin. Clin. Cancer Res. 6:4658-62
6. Devita, Hellman, and Rosenburg (2008). Cancer: Principles and Practice of Oncology. 8^{th} Edition.
7. Dudley M E, Wunderlich J R, Robbins P F, Yang J C, Hwu P, Schwartzentruber D J, Topalian S L, Sherry R, Restifo N P, Hubicki A M, Robinson M R, Raffeld M, Duray P, Seipp C A, Rogers-Freezer L, Morton K E, Mavroukakis S A, White D E, Rosenberg S A (2002) Cancer regression and autoimmunity in patients after clonal repopulation with antitumor lymphocytes. Science. 298(5594):850-854.
8. Eisenhauer E A, Therasse P, Bogaerts J, Schwartz L H, Sargent D, Ford R, Dancey J, Arbuck S, Gwyther S, Mooney M, Rubinstein L, Shankar L, Dodd L, Kaplan R, Lacombe D, Verweij J. (2009) New Response Evaluation Criteria in Solid Tumours: Revised RECIST Guideline (Version 1.1), European Journal of Cancer. 45:228-247.
9. Finn O J (2003) Cancer vaccines: between the idea and the reality. Nat Rev Immunol. 3(8):630-641.
10. Gilewski T, Ragupathi G, Bhuta S, Williams L J, Musselli C, Zhang X F, Bornmann W G, Spassova M, Bencsath K P, Panageas K S, Chin J, Hudis C A, Norton L, Houghton A N, Livingston P O, Danishefsky S J. (2001) Immunization of metastatic breast cancer patients with a fully synthetic globo H conjugate: a phase I trial. Proc Natl Acad Sci USA 98:3270-5
11. Helling F, Shang A, Calves M, Zhang S, Ren S, Yu R K, Oettgen H F, Livingston P O (1994) Increased immunogenicity of GD3 conjugate vaccines: comparison of various carrier proteins and selection of GD3-KLH for further testing. Cancer Res. 54: 197-203.
12. Helling F, Zhang S, Shang A, Adluri S, Calves M, Koganty R, Longenecker B M, Yao T-J, Oettgen H F, Livingston P O (1995) G_{M2}-KLH conjugate vaccine: increased immunogenicity in melanoma patients after administration with immunological adjuvant QS-21. Cancer Res. 55: 2783-2788.
13. Ho C, Lee K, Shiah H, et al (2020) A phase I cohort expansion trial of OBI-833 in non-small cell lung cancer patients. Ann Oncol. 31(suppl_6):51386-406.
14. Huang Y L, Hung J T, Cheung S K, Lee H Y, Chu K C, Li S T, Lin Y C, Ren C T, Cheng T J, Hsu T L, Yu A L, Wu C Y, Wong C H (2013). Carbohydrate-based vaccines with a glycolipid adjuvant for breast cancer. Proc. Natl. Acad. Sci. 110: 2517-2522.
15. Huang C S, Yu A L, Tseng L M, Chow L W C, Hou M F, Hurvitz S A, Schwab R B, L Murray J, Chang H K, Chang H T, Chen S C, Kim S B, Hung J T, Ueng S H, Lee S H, Chen C C, Rugo H S (2020) Globo H-KLH vaccine adagloxad simolenin (OBI-822)/OBI-821 in patients with metastatic breast cancer: phase II randomized, placebo-controlled study. J Immunother Cancer. 8(2):e000342.
16. Ibrahim N K, Murray J L, Zhou D, Mittendorf E A, Sample D, Tautchin M, Miles D (2013) Survival advantage in patients with metastatic breast cancer receiving endocrine therapy plus sialyl Tn-KLH vaccine: post hoc analysis of a large randomized trial. J. Cancer 4: 577-584.
17. Khong H T and Restifo N P (2002) Natural selection of tumor variants in the generation of “tumor escape” phenotypes. Nat. Immunol. 3: 999-1005.
18. Livingston P O (1995a) Augmenting the immunogenicity of carbohydrate tumor antigens. Seminars in Can. Biol. 6:357-366.
19. Livingston P O (1995b) Approaches to augmenting the immunogenicity of melanoma gangiosides: from whole melanoma cells to gangioside-KHL conjugate vaccines. Immunol. Reviews 145:148-166.
20. Livingston P O (1998) Ganglioside vaccines with emphasis on G_{M2}. Semin. Oncol. 25: 636-645.
21. Livingston P O, Adluri S, Helling F, Yao T-J, Kensil C R, Newman M J, and Marciani D (1994a) Phase 1 trial of immunological adjuvant QS-21 with a GM2 ganglioside-keyhole limpet haemocyanin conjugate vaccine in patients with malignant melanoma. Vaccine 12:1275-1280.
22. Livingston P O, Wong GYC, Adluri S, Tao Y, Padavan M, Parente R, Hanlon C, Jones Calves M, Helling F, Ritter G, Oettgen H F, Old L J (1994b) Improved survival in stage III melanoma patients with GM2 antibodies: a randomized trial of adjuvant vaccination with GM2 ganglioside. J. Clin. Oncol. 12: 1036-1044.
23. Livingston P O, Zhang S, Lloyd K O (1997) Carbohydrate vaccines that induce antibodies against cancer. 1. Rationale. Cancer Immunol. Immunother. 45: 1-9.
24. Livingston P O & Ragupathi G. (1997) Carbohydrate vaccines that induce antibodies against cancer. 2. Previous experience and future plans. Cancer Immunol. Immunother. 45: 10-19.
25. Longenecker B M, Reddish M, Koganty R, and MacLean G D (1993) Immune responses of mice and human breast cancer patients following immunization with synthetic sialyl-Tn conjugated to KLH plus detox adjuvant. Ann. N. Y. Acad. Sci. 690: 276-91
26. Longenecker B M, Reddish M, Koganty R, and MacLean G D (1994) Specificity of the IgG response in mice and human breast cancer patients following immunization against synthetic sialyl-Tn, an epitope with possible functional significance in metastasis. Adv. Exp. Med. Biol. 353: 105-24.
27. Ludewig B, Ochsenbein A F, Odermatt B, Paulin D, Hengartner H, Zinkernagel R M (2000) Immunotherapy with dendritic cells directed against tumor antigens shared with normal host cells results in severe autoimmune disease. J Exp Med. 6; 191(5):795-804.
28. Lou Y W, Wang P Y, Yeh S C, Chuang P K, Li S T, Wu C Y, Khoo K H, Hsiao M, Hsu T L, Wong C H (2014) Stage-specific embryonic antigen-4 as a potential therapeutic target in glioblastoma multiforme and other cancers. Proc. Natl. Acad. Sci. 111:2482-2487.
29. MacLean G D, Reddish M, Koganty R R, Wong T, Gandhi S, Smolenski M, Samuel J, Nabholtz J M, Longenecker B M (1993) Immunization of breast cancer patients using a synthetic sialyl-Tn glycoconjugate plus Detox adjuvant. Cancer Immunol. Immunother. 36: 215-22
30. McCaffery M, Yao T-J, Williams L, Livingston P O, Houghton A N, Chapman P B (1996) Enhanced immunogenicity of BEC2 anti-idiotypic monoclonal antibody that mimics GD3 ganglioside when combined with adjuvant. Clin. Cancer Res. 2: 679-686.
31. Menard S, Tagliabue E, Canevari S, Fossati G, Colnaghni M I (1983) Generation of monoclonal antibodies reacting with normal and cancer cells of human breast. Cancer Res. 43:1295-1300.
32. Miotti S, Leoni F, Canevari S, Sonnino S, Colnaghi M I (1989) Immunoblotting detection of carbohydrate epitopes in glycolipids and glycoproteins of tumor origin. In: Oettgen H F, ed. Gangliosides and Cancer. Weinheim: VCH Verlagsgesellschaft, pp 169-176.
33. Musselli C, Livingston P O, Ragupathi G (2001) Keyhole limpet hemocyanin conjugate vaccines against cancer: the Memorial Sloan Kettering experience. J. Cancer Res. Clin. Oncol. 127 Suppl 2:R20-6
34. Overwijk W W, Lee D S, Surman D R, Irvine K R, Touloukian C E, Chan C C, Carroll M W, Moss B, Rosenberg S A, Restifo N P (1999) Vaccination with a recombinant vaccinia virus encoding a “self” antigen induces autoimmune vitiligo and tumor cell destruction in mice: requirement for CD4(+) T lymphocytes. Proc Natl Acad Sci USA. 16; 96(6):2982-7.
35. Pawelec G, Zeuthen J, Kiessling R (1997) Escape from host-antitumor immunity. Crit. Rev. Oncog. 8: 111-41
36. Rabinovich N R, McInnes P, Klein D L, Hall B F. (1994) Vaccine technologies: view to future. Science 265: 1401-1404
37. Ragupathi G, Meyers M, Adluri S, Howard L, Musselli L, Livingston P O (2000) Induction of antibodies against GD3 ganglioside in melanoma patients by vaccination with G_{D3}-lactone-KLH conjugate plus immunological adjuvant QS-21. Int. J. Cancer 85: 659-666.
38. Ragupathi, G., Park, T. K., Zhang, S., Kim, I. J., Graber, L., Adluri, R., Lloyd, K. O., Danishefsky, S. J. & Livingston, P. O. (1997) Immunization of mice with a fully synthetic globo H antigen results in antibodies against human cancer cells: A combined chemical-immunological approach to the fashioning of an anticancer vaccine. Angew. Chem. Int. Ed. Engl. 36: 125-128
39. Sandmaier B M, Oparin D V, Holmberg L A, Reddish M A, MacLean G D, Longenecker B M (1999) Evidence of a cellular immune response against sialyl-Tn in breast and ovarian cancer patients after high-dose chemotherapy, stem cell rescue, and immunization with Theratope STn-KLH cancer vaccine. J. Immunother. 22: 54-66.
40. Slovin S F, Ragupathi G, Adluri S, Ungers G, Terry K, Kim S, Spassova M, Bornmann W G, Fazzari M, Dantis L, Olkiewicz K, Lloyd K O, Livingston P O, Danishefsky S J, Scher H I. (1999) Carbohydrate vaccines in cancer: immunogenicity of a fully synthetic globo H hexasaccharide conjugate in man. Proc Natl Acad Sci USA 96:5710-5
41. Shiah H, Strauss J F, Lin C, et al (2020) OBI-833 was safe and immunogenic, without treatment-related SAEs, in a phase I dose-escalation trial. Ann Oncol. 31(suppl_6):51270-2.
42. Sotomayor E M, Borrello I, Levitsky H I (1996) Tolerance and cancer: a critical issue in tumor immunology. Crit. Rev. Oncog. 7:433-56.
43. White A C, Cloutier P, Coughlin R T (1991) A purified saponin acts as an adjuvant for a T-independent antigen. Adv. Exp. Med. Biol. 303: 207-210.
44. Yu A L, Gilman A L, Ozkaynak M F, London W B, Kreissman S G, Chen H X, Smith M, Anderson B, Villablanca J G, Matthay K K, Shimada H, Grupp S A, Seeger R, Reynolds C P, Buxton A, Reisfeld R A, Gillies S D, Cohn S L, Maris J M, Sondel P M. (2010) Anti-GD2 Antibody with G M-CSF, IL2 and Isotretinoin for Neuroblastoma. New Eng. J. Med. 363:1324-34.
45. Zhang S, Graeber L A. Helling F, Ragupathi G, Adluri S, Lloyd K O, Livingston P O (1996) Augmenting the immunogenicity of synthetic MUC1 peptide vaccine in mine. Cancer Res. 56:3315-3319.
46. Zhang, S., Cordon-Cardo, C., Zhang, H. S., Reuter, V. E., Adluri, S., Hamilton, W. B., Lloyd, K. O. & Livingston, P. O. (1997a) Selection of tumor antigens as targets for immune attack using immunohistochemistry: I. Focus on gangliosides. Int. J. Cancer 73, 42-49.
47. Zhang S, Zhang H S, Cordon-Cardo C, Reiter V S, Singhal A K, Lloyd K O, Livingston P O. (1997b) Selection of tumor antigens as targets for immune attack using immunohistochemistry: II. Blood group-related antigens. Int. J. Cancer, 73, 50-56.
48. Zhang, S., Zhang, H. S., Reuter, V. E., Slovin, S. F., Scher, H. I. & Livingston, P. (1998) Expression of potential target antigens for immunotherapy on primary and metastatic prostate cancers. Clin. Cancer Res. 4, 295-302.
49. Guidance for Industry-Clinical Considerations for Therapeutic Cancer Vaacines. U S Department of Health and Human Services, FDA, CBER, October 2011.
50. Zhang T, de Waard A A, Wuhrer M, Spaapen R M. (2019) The Role of Glycosphingolipids in Immune Cell Functions. Front Immunol 10, 90.

While specific aspects of the invention have been described and illustrated, such aspects should be considered illustrative of the invention only and not as limiting the invention as construed in accordance with the accompanying claims. All publications and patent applications cited in this specification are herein incorporated by reference in their entirety for all purposes as if each individual publication or patent application were specifically and individually indicated to be incorporated by reference in its entirety for all purposes. Although the foregoing invention has been described in some detail by way of illustration and example for purposes of clarity of understanding, it will be readily apparent to one of ordinary skill in the art in light of the teachings of this invention that certain changes and modifications can be made thereto without departing from the spirit or scope of the appended claims.

What is claimed is:

1. A method for treating or preventing advanced gastric, lung, colorectal, or breast cancer in patient comprising administering a therapeutically effective dose of a Globo H-DT-CRM197 (diphtheria toxin cross-reacting material 197) glycoconjugate.

2. (canceled)

3. (canceled)

4. (canceled)

5. The method of claim 1, wherein the Globo H-DT-CRM197 conjugate vaccine is administered as a pharmaceutical composition.

6. The method of claim 5, wherein the pharmaceutical composition comprises an OBI-833 vaccine and an OBI-821 adjuvant.

7. (canceled)

8. The method of claim 1, wherein the therapeutically effective dose is less than 1000 μg.

9. The method of claim 1, wherein the administration procedure comprises intramuscular injection, subcutaneous injection, intravenous injection, intraperitoneal injection, intra-arterial injection, intrasynovial injection, or intra-pleural injection.

10. The method of claim 1, wherein the vaccine is administered intermittently at a time interval of one week to five years or more.

11. The method of claim 1, wherein the vaccine is administered once every one week, once every two weeks, once every three weeks, once every four weeks, once every five weeks, once every six weeks, once every seven weeks, once every eight weeks, once every nine weeks, once every ten weeks, once every eleven weeks or once every twelve weeks.

12. (canceled)

13. The method of claim 1, wherein the Globo H-DT-CRM197 conjugate vaccine is administered to the patient in combination with one or more anti-proliferative agents.

14. The method of claim 13, wherein the anti-proliferative agent is selected from cyclophosphamide, opiate, granulocyte colony-stimulating factor (GCSF), estrogen inhibitors, aromatase inhibitors, pituitary downregulators, tamoxifen selective estrogen-receptor modulator, rolaxifene, estrogen receptor down-regulator, anticoagulant, enzyme, hematopoietic growth factor, anti-neoplastic agent, antimetabolites, miscellaneous cytotoxic agents, vinca alkaloid, epipodophyllotoxins, alkylating agents, taxanes, antitumor antibiotics, camptothecins, nitrosoureas, HER1/EGFR tyrosine kinase inhibitor, VEGF protein inhibitor, HER-2/ErbB2 inhibitor, interferon, interleukin, monoclonal antibody, glucocorticoid steroid, gefitinib, icotininib, erlotinib, osimertinib, afatinib, dacomitinib, rociletinib, olmutinib, almonertinib, alflutinib, AC0010, BPI-7711, tarloxitinib, TAK-788, EAI045, BLU-945, nazartinib, naquotinib, mavelertinib, poziotinib, DBPR112, docetaxel, gemcitabine, cisplatin; carboplatin; paclitaxel, trastuzumab, temozolomide, tamoxifen, doxorubicin, oxaliplatin, bortezomib, sutent, letrozole, imatinib mesylate, MEK inhibitor, fulvestrant, leucovorin (folinic acid); rapamycin, lapatinib, lonafarnib, sorafenib, gefitinib, irinotecan, tipifarnib, Cremophor-free, paclitaxel, vandetanib, chloranmbucil, temsirolimus, pazopanib, canfosfamide, thiotepa, cyclosphosphamide, 5-fluorouracil (5-FU), vinorelbine, novantrone, teniposide, edatrexate, daunomycin, aminopterin, capecitabine, ibandronate, topoisomerase inhibitor RFS 2000, difluoromethylornithine (DMFO), tamoxifen, raloxifene, droloxifene, 4-hydroxytamoxifen, trioxifene, keoxifene, onapristone, toremifine citrate, 4(5)-imidazoles, aminoglutethimide, megestrol acetate, exemestane, formestanie, fadrozole, vorozole, letrozole, anastrozole, flutamide, nilutamide, bicalutamide, leuprolide, goserelin, troxacitabine (α-1,3-dioxolane nucleoside cytosine analog), lipid kinase inhibitor, oblimersen, angiozyme, allovectin, leuvectin, vaxid, aldesleukin, lurtotecan, abarelix, bevacizumab, alemtuzumab, bevacizumab, cetuximab, panitumumab, rituximab, pertuzumab, trastuzumab, tositumomab, gemtuzumab or ozogamicin.

15. The method of claim 1, wherein the said method further comprises reducing tumor volume and/or improving survival of the subject by modulating Globo series antigens interaction.

16. The method of claim 15, wherein the survival comprises overall survival (OS) and/or progression-free survival (PFS).

17. The method of claim 15, wherein the modulation of Globo series antigens interaction further comprises one or more of the following:
(a) Induction of Antibody-Dependent Cellular Cytotoxicity (ADCC) and Complement-Dependent Cytotoxicity (CDC) for tumor killing; or
(b) Induction of anti-Globo series antigens IgM/IgG immune response to elicit Antibody-Dependent Cellular Cytotoxicity (ADCC) and Complement-Dependent Cytotoxicity (CDC)-mediated tumor cell killing;
wherein the Globo series antigens are selected from a group consisting of Globo H, SSEA-3, and SSEA-4.

18. A method for inducing/enhancing an immune response in a subject, comprising administering to the subject an immunogenic agent.

19. The method of claim 18, wherein the immunogenic agent is Globo-H-DT-CRM197 conjugate.

20. (canceled)

21. (canceled)

22. The method of claim 18, wherein the immunogenic agent is OBI-833 and related variants.

23. The method of claim 18, wherein the subject is human.

24. (canceled)

25. The method of claim 18, wherein the immune response comprises IgG, IgM, or B cell/T cell-mediated response.

26. A method for treating or preventing lung cancer comprising administering to a patient in need thereof a therapeutically effective dose of Globo H-DT CRM197 (diphtheria toxin cross-reacting material 197) glycoconjugate vaccine by a route selected from:
(a) Administering vaccine two or more times (e.g., 3, 4, 5, 6, 7, 8, 9, 10, 11, 12 or more times);
(b) Adjusting time interval and/or dosing amount regimen between two successive administrations;
(c) Adjusting routes of administration and/or altering injection locations of administration; or
(d) Any combination of the above thereby each administration increases antibody immune response and/or increases antigen-antibody binding affinity.

27. The method of claim 26, wherein the injection can be altered and/or supplemented by the addition of immune response booster agents.

28. (canceled)

29. The method of claim 28, wherein the immune response comprises IgG, IgM, or B cell/T cell-mediated response.

30. (canceled)

31. The method of claim 26, wherein the lung cancer is small cell lung cancer (SCLC) or non-small cell lung cancer (NSCLC).

32. The method of claim 26, wherein the vaccine is OBI-833 and further comprises OBI-821 adjuvant,

33. (canceled)

34. (canceled)

35. (canceled)

36. (canceled)

37. (canceled)

38. (canceled)

39. (canceled)

40. (canceled)
